SEQUENCE LISTING (103 sequences)

SEQ ID NO: 1 - protein, 349 aa, Homo sapiens
GRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLG

SEQ ID NO: 2 - protein, 701 aa, Homo sapiens
LSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 3 - protein, 883 aa, Homo sapiens
MQKIMHISVLLSPVLWGLIFGVSSNSIQIGGLFPRGADQEYSAFRVGMVQFSTSEFRLTPHIDNLEVANSFAVTNAFCSQFSRGVYAIFGFYDKKSVNTITSFCGTLHVSFITPSFPTDGTHPFVIQMRPDLKGALLSLIEYYQWDKFAYLYDSDRGLSTLQAVLDSAAEKKWQVTAINVGNINNDKKDEMYRSLFQDLELKKERRVILDCERDKVNDIVDQVITIGKHVKGYHYIIANLGFTDGDLLKIQFGGANVSGFQIVDYDDSLVSKFIERWSTLEEKEYPGAHTTTIKYTSALTYDAVQVMTEAFRNLRKQRIEISRRGNAGDCLANPAVPWGQGVEIERALKQVQVEGLSGNIKFDQNGKRINYTINIMELKTNGPRKIGYWSEVDKMVVTLTELPSGNDTSGLENKTVVVTTILESPYVMMKKNHEMLEGNERYEGYCVDLAAEIAKHCGFKYKLTIVGDGKYGARDADTKIWNGMVGELVYGKADIAIAPLTITLVREEVIDFSKPFMSLGISIMIKKPQKSKPGVFSFLDPLAYEIWMCIVFAYIGVSVVLFLVSRFSPYEWHTEEFEDGRETQSSESTNEFGIFNSLWFSLGAFMQQGCDISPRSLSGRIVGGVWWFFTLIIISSYTANLAAFLTVERMVSPIESAEDLSKQTEIAYGTLDSGSTKEFFRRSKIAVFDKMWTYMRSAEPSVFVRTTAEGVARVRKSKGKYAYLLESTMNEYIEQRKPCDTMKVGGNLDSKGYGIATPKGSSLRNAVNLAVLKLNEQGLLDKLKNKWWYDKGECGSGGGDSKEKTSALSLSNVAGVFYILVGGLGLAMLVALIEFCYKSRAEAKRMKVAKNAQNINPSSSQNSQNFATYKEGYNVYGIESVKI

SEQ ID NO: 4 - RNA, 2145 nt, Artificial Sequence
  source: /note=“Description of Artificial Sequence Synthetic polynucleotide”
augggcaggagcaggcuuuuggaagauuuucgaaacaaccgcuaccccaauuuacaacugcgggagauugcuggacauauaauggaauuuucccaagaccagcauggguccagauucauucagcugaaacuggagcgugccacaccagcugagcgccagcuugucuucaaugaaauccuccaggcugccuaccaacucaugguggauguguuugguaauuacgucauucagaaguucuuugaauuuggcagucuugaacagaagcuggcuuuggcagaacggauucgaggccacguccugucauuggcacuacagauguauggcugccguguuauccagaaagcucuugaguuuauuccuucagaccagcagaaugagaugguucgggaacuagauggccaugucuugaagugugugaaagaucagaauggcaaucacgugguucagaaaugcauugaauguguacagccccagucuuugcaauuuaucaucgaugcguuuaagggacagguauuugccuuauccacacauccuuauggcugccgagugauucagagaauccuggagcacugucucccugaccagacacucccuauuuuagaggagcuucaccagcacacagagcagcuuguacaggaucaauauggaaauuauguaauccaacauguacuggagcacggucguccugaggauaaaagcaaaauuguagcagaaauccgaggcaauguacuuguauugagucagcacaaauuugcaagcaauguuguggagaaguguguuacucacgccucacguacggagcgcgcugugcucaucgaugaggugugcaccaugaacgacgguccccacagugccuuauacaccaugaugaaggaccaguaugccaacuacgugguccagaagaugauugacguggcggagccaggccagcggaagaucgucaugcauaagauccggccccacaucgcaacucuucguaaguacaccuauggcaagcacauucuggccaagcuggagaaguacuacaugaagaacgguguugacuuagggggagguggcggaucgggagguggcggaucgggagguggcggaucgggcaggagcaggcuuuuggaagauuuucgaaacaaccgcuaccccaauuuacaacugcgggagauugcuggacauauaauggaauuuucccaagaccagcauggguccagauucauucagcugaaacuggagcgugccacaccagcugagcgccagcuugucuucaaugaaauccuccaggcugccuaccaacucaugguggauguguuugguaauuacgucauucagaaguucuuugaauuuggcagucuugaacagaagcuggcuuuggcagaacggauucgaggccacguccugucauuggcacuacagauguauggcugccguguuauccagaaagcucuugaguuuauuccuucagaccagcagaaugagaugguucgggaacuagauggccaugucuugaagugugugaaagaucagaauggcaaucacgugguucagaaaugcauugaauguguacagccccagucuuugcaauuuaucaucgaugcguuuaagggacagguauuugccuuauccacacauccuuauggcugccgagugauucagagaauccuggagcacugucucccugaccagacacucccuauuuuagaggagcuucaccagcacacagagcagcuuguacaggaucaauauggaaauuauguaauccaacauguacuggagcacggucguccugaggauaaaagcaaaauuguagcagaaauccgaggcaauguacuuguauugagucagcacaaauuugcaagcaauguuguggagaaguguguuacucacgccucacguacggagcgcgcugugcucaucgaugaggugugcaccaugaacgacgguccccacagugccuuauacaccaugaugaaggaccaguaugccaacuacgugguccagaagaugauugacguggcggagccaggccagcggaagaucgucaugcauaagauccggccccacaucgcaacucuucguaaguacaccuauggcaagcacauucuggccaagcuggagaaguacuacaugaagaacgguguugacuuaggguga

SEQ ID NO: 5 - RNA, 1215 nt, Artificial Sequence
  source: /note=“Description of Artificial Sequence Synthetic polynucleotide”
augcuccaccucgaccaaacacccagcagacagccuaucccuuccgaaggacugcagcugcauuuaccgcagguuuuagcugacgcugucucacgccugguccuggguaaguuuggugaucugaccgacaacuucuccuccccucacgcucgcagaaaagugcuggcuggagucgucaugacaacaggcacagauguuaaagaugccaaggugauaaguguuucuacaggaggcaaauguauuaauggugaauacaugagugaucguggccuugcauuaaaugacugccaugcagaaauaauaucucggagauccuugcucagauuucuuuauacacaacuugagcuuuacuuaaauaacaaagaugaucaaaaaagauccaucuuucagaaaucagagcgagggggguuuaggcugaaggagaauguccaguuucaucuguacaucagcaccucucccuguggagaugccagaaucuucucaccacaugagccaauccuggaagaaccagcagauagacacccaaaucguaaagcaagaggacagcuacggaccaaaauagagucuggucaggggacgauuccagugcgcuccaaugcgagcauccaaacgugggacggggugcugcaaggggagcggcugcucaccauguccugcagugacaagauugcacgcuggaacguggugggcauccagggaucacugcucagcauuuucguggagcccauuuacuucucgagcaucauccugggcagccuuuaccacggggaccaccuuuccagggccauguaccagcggaucuccaacauagaggaccugccaccucucuacacccucaacaagccuuugcucaguggcaucagcaaugcagaagcacggcagccagggaaggcccccaacuucagugucaacuggacgguaggcgacuccgcuauugaggucaucaacgccacgacugggaaggaugagcugggccgcgcgucccgccuguguaagcacgcguuguacugucgcuggaugcgugugcacggcaagguucccucccacuuacuacgcuccaagauuaccaagcccaacguguaccaugaguccaagcuggcggcaaaggaguaccaggccgccaaggcgcgucuguucacagccuucaucaaggcggggcugggggccuggguggagaagcccaccgagcaggaccaguucucacucacgccuuga

SEQ ID NO: 6 - RNA, 3573 nt, Artificial Sequence
  source: /note=“Description of Artificial Sequence Synthetic polynucleotide”
auggacuauaaggaccacgacggagacuacaaggaucaugauauugauuacaaagacgaugacgauaagauggccccaaagaagaagcggaaggucgguauccacggagucccagcagcccuccaccucgaccaaacacccagcagacagccuaucccuuccgaaggacugcagcugcauuuaccgcagguuuuagcugacgcugucucacgccugguccuggguaaguuuggugaucugaccgacaacuucuccuccccucacgcucgcagaaaagugcuggcuggagucgucaugacaacaggcacagauguuaaagaugccaaggugauaaguguuucuacaggaggcaaauguauuaauggugaauacaugagugaucguggccuugcauuaaaugacugccaugcagaaauaauaucucggagauccuugcucagauuucuuuauacacaacuugagcuuuacuuaaauaacaaagaugaucaaaaaagauccaucuuucagaaaucagagcgagggggguuuaggcugaaggagaauguccaguuucaucuguacaucagcaccucucccuguggagaugccagaaucuucucaccacaugagccaauccuggaagaaccagcagauagacacccaaaucguaaagcaagaggacagcuacggaccaaaauagagucuggucaggggacgauuccagugcgcuccaaugcgagcauccaaacgugggacggggugcugcaaggggagcggcugcucaccauguccugcagugacaagauugcacgcuggaacguggugggcauccagggaucacugcucagcauuuucguggagcccauuuacuucucgagcaucauccugggcagccuuuaccacggggaccaccuuuccagggccauguaccagcggaucuccaacauagaggaccugccaccucucuacacccucaacaagccuuugcucaguggcaucagcaaugcagaagcacggcagccagggaaggcccccaacuucagugucaacuggacgguaggcgacuccgcuauugaggucaucaacgccacgacugggaaggaugagcugggccgcgcgucccgccuguguaagcacgcguuguacugucgcuggaugcgugugcacggcaagguucccucccacuuacuacgcuccaagauuaccaagcccaacguguaccaugaguccaagcuggcggcaaaggaguaccaggccgccaaggcgcgucuguucacagccuucaucaaggcggggcugggggccuggguggagaagcccaccgagcaggaccaguucucacucacgccuggagguggcggaucgggagguggcggaucgggagguggcggaucgggcaggagcaggcuuuuggaagauuuucgaaacaaccgcuaccccaauuuacaacugcgggagauugcuggacauauaauggaauuuucccaagaccagcauggguccagauucauucagcugaaacuggagcgugccacaccagcugagcgccagcuugucuucaaugaaauccuccaggcugccuaccaacucaugguggauguguuugguaauuacgucauucagaaguucuuugaauuuggcagucuugaacagaagcuggcuuuggcagaacggauucgaggccacguccugucauuggcacuacagauguauggcugccguguuauccagaaagcucuugaguuuauuccuucagaccagcagaaugagaugguucgggaacuagauggccaugucuugaagugugugaaagaucagaauggcaaucacgugguucagaaaugcauugaauguguacagccccagucuuugcaauuuaucaucgaugcguuuaagggacagguauuugccuuauccacacauccuuauggcugccgagugauucagagaauccuggagcacugucucccugaccagacacucccuauuuuagaggagcuucaccagcacacagagcagcuuguacaggaucaauauggaaauuauguaauccaacauguacuggagcacggucguccugaggauaaaagcaaaauuguagcagaaauccgaggcaauguacuuguauugagucagcacaaauuugcaagcaauguuguggagaaguguguuacucacgccucacguacggagcgcgcugugcucaucgaugaggugugcaccaugaacgacgguccccacagugccuuauacaccaugaugaaggaccaguaugccaacuacgugguccagaagaugauugacguggcggagccaggccagcggaagaucgucaugcauaagauccggccccacaucgcaacucuucguaaguacaccuauggcaagcacauucuggccaagcuggagaaguacuacaugaagaacgguguugacuuagggggagguggcggaucgggagguggcggaucgggagguggcggaucgggcaggagcaggcuuuuggaagauuuucgaaacaaccgcuaccccaauuuacaacugcgggagauugcuggacauauaauggaauuuucccaagaccagcauggguccagauucauucagcugaaacuggagcgugccacaccagcugagcgccagcuugucuucaaugaaauccuccaggcugccuaccaacucaugguggauguguuugguaauuacgucauucagaaguucuuugaauuuggcagucuugaacagaagcuggcuuuggcagaacggauucgaggccacguccugucauuggcacuacagauguauggcugccguguuauccagaaagcucuugaguuuauuccuucagaccagcagaaugagaugguucgggaacuagauggccaugucuugaagugugugaaagaucagaauggcaaucacgugguucagaaaugcauugaauguguacagccccagucuuugcaauuuaucaucgaugcguuuaagggacagguauuugccuuauccacacauccuuauggcugccgagugauucagagaauccuggagcacugucucccugaccagacacucccuauuuuagaggagcuucaccagcacacagagcagcuuguacaggaucaauauggaaauuauguaauccaacauguacuggagcacggucguccugaggauaaaagcaaaauuguagcagaaauccgaggcaauguacuuguauugagucagcacaaauuugcaagcaauguuguggagaaguguguuacucacgccucacguacggagcgcgcugugcucaucgaugaggugugcaccaugaacgacgguccccacagugccuuauacaccaugaugaaggaccaguaugccaacuacgugguccagaagaugauugacguggcggagccaggccagcggaagaucgucaugcauaagauccggccccacaucgcaacucuucguaaguacaccuauggcaagcacauucuggccaagcuggagaaguacuacaugaagaacgguguugacuuagggagcggcggcaagcggcccgccgccaccaagaaggccggccaggccaagaagaagaaguga

SEQ ID NO: 7 - RNA, 2145 nt, Artificial Sequence
  source: /note=“Description of Artificial Sequence Synthetic polynucleotide”
augggccgcagccgccuuuuggaagauuuucgaaacaaccgguaccccaauuuacaacugcgggagauugccggacauauaauggaauuuucccaagaccagcauggguccagauucauucgccugaaacuggagcgugccacaccagcugagcgccagcuugucuuuaaugaaauccuccaggcugccuaccaacucaugguggauguguuugguaguuacgucauugagaaguucuuugaauuuggcagucuugaacagaagcuggcuuuggcagaacggauucgaggucacguccugucauuggcacuacagauguauggcugccguguuauccagaaagcucuugaguuuauuccuucagaccagcagaaugagaugguucgggaacuagauggccaugucuugaagugugugaaagaucagaauggcugucacgugguucagaaaugcauugaauguguacagccccagucuuugcaauuuaucaucgaugcguuuaagggccagguauuugccuuauccacacauccuuauggcucccgagugauugagagaauccuggagcacugucucccugaccagacacucccuauuuuagaggagcuucaccagcacacagagcagcuuguacaggaucaauauggauguuauguaauccaacauguacuggagcacggucguccugaggauaaaagcaaaauuguagcagaaauccgaggcaauguacuuguauugagucagcacaaauuugcaugcaauguugugcagaaguguguuacucacgccucacguacggagcgcgcugugcucaucgaugaggugugcaccaugaacgacgguccccacagugccuuauacaccaugaugaaggaccaguaugccagcuacgugguccgcaagaugauugacguggcggagccaggccagcggaagaucgucaugcauaagauccgaccccacaucgcaacucuucguaaguacaccuauggcaagcacauucuggccaagcuggagaaguacuacaugaagaacgguguugacuuagggggagguggcggaucgggagguggcggaucgggagguggcggaucgggccgcagccgccuuuuggaagauuuucgaaacaaccgguaccccaauuuacaacugcgggagauugccggacauauaauggaauuuucccaagaccagcaugggaacagauucauucagcugaaacuggagcgugccacaccagcugagcgccagcuugucuuuaaugaaauccuccaggcugccuaccaacucaugguggauguguuugguuguuacgucauucagaaguucuuugaauuuggcagucuugaacagaagcuggcuuuggcagaacggauucgaggucacguccugucauuggcacuacagauguauggcucccguguuaucgagaaagcucuugaguuuauuccuucagaccagcagaaugagaugguucgggaacuagauggccaugucuugaagugugugaaagaucagaauggcaaucacgugguucagaaaugcauugaauguguacagccccagucuuugcaauuuaucaucgaugcguuuaagggacagguauuugccuuauccacacauccuuauggcugccgagugauucagagaauccuggagcacugucucccugaccagacacucccuauuuuagaggagcuucaccagcacacagagcagcuuguacaggaucaauauggaaguuauguaauccgccauguacuggagcacggucguccugaggauaaaagcaaaauuguagcagaaauccgaggcaauguacuuguauugagucagcacaaauuugcaaacaauguugugcagaaguguguuacucacgccucacguacggagcgcgcugugcucaucgaugaggugugcaccaugaacgacgguccccacagugccuuauacaccaugaugaaggaccaguaugccugcuacgugguccagaagaugauugacguggcggagccaggccagcggaagaucgucaugcauaagauccgaccccacaucgcaacucuucguaaguacaccuauggcaagcacauucuggccaagcuggagaaguacuacaugaagaacgguguugacuuaggguga

SEQ ID NO: 8 - RNA, 3573 nt, Artificial Sequence
  source: /note=“Description of Artificial Sequence Synthetic polynucleotide”
auggacuauaaggaccacgacggagacuacaaggaucaugauauugauuacaaagacgaugacgauaagauggccccaaagaagaagcggaaggucgguauccacggagucccagcagcccuccaccucgaccaaacacccagcagacagccuaucccuuccgaaggacugcagcugcauuuaccgcagguuuuagcugacgcugucucacgccugguccuggguaaguuuggugaucugaccgacaacuucuccuccccucacgcucgcagaaaagugcuggcuggagucgucaugacaacaggcacagauguuaaagaugccaaggugauaaguguuucuacaggaggcaaauguauuaauggugaauacaugagugaucguggccuugcauuaaaugacugccaugcagaaauaauaucucggagauccuugcucagauuucuuuauacacaacuugagcuuuacuuaaauaacaaagaugaucaaaaaagauccaucuuucagaaaucagagcgagggggguuuaggcugaaggagaauguccaguuucaucuguacaucagcaccucucccuguggagaugccagaaucuucucaccacaugagccaauccuggaagaaccagcagauagacacccaaaucguaaagcaagaggacagcuacggaccaaaauagagucuggucaggggacgauuccagugcgcuccaaugcgagcauccaaacgugggacggggugcugcaaggggagcggcugcucaccauguccugcagugacaagauugcacgcuggaacguggugggcauccagggaucacugcucagcauuuucguggagcccauuuacuucucgagcaucauccugggcagccuuuaccacggggaccaccuuuccagggccauguaccagcggaucuccaacauagaggaccugccaccucucuacacccucaacaagccuuugcucaguggcaucagcaaugcagaagcacggcagccagggaaggcccccaacuucagugucaacuggacgguaggcgacuccgcuauugaggucaucaacgccacgacugggaaggaugagcugggccgcgcgucccgccuguguaagcacgcguuguacugucgcuggaugcgugugcacggcaagguucccucccacuuacuacgcuccaagauuaccaagcccaacguguaccaugaguccaagcuggcggcaaaggaguaccaggccgccaaggcgcgucuguucacagccuucaucaaggcggggcugggggccuggguggagaagcccaccgagcaggaccaguucucacucacgccuggagguggcggaucgggagguggcggaucgggagguggcggaucgggccgcagccgccuuuuggaagauuuucgaaacaaccgguaccccaauuuacaacugcgggagauugccggacauauaauggaauuuucccaagaccagcauggguccagauucauucgccugaaacuggagcgugccacaccagcugagcgccagcuugucuuuaaugaaauccuccaggcugccuaccaacucaugguggauguguuugguaguuacgucauugagaaguucuuugaauuuggcagucuugaacagaagcuggcuuuggcagaacggauucgaggucacguccugucauuggcacuacagauguauggcugccguguuauccagaaagcucuugaguuuauuccuucagaccagcagaaugagaugguucgggaacuagauggccaugucuugaagugugugaaagaucagaauggcugucacgugguucagaaaugcauugaauguguacagccccagucuuugcaauuuaucaucgaugcguuuaagggccagguauuugccuuauccacacauccuuauggcucccgagugauugagagaauccuggagcacugucucccugaccagacacucccuauuuuagaggagcuucaccagcacacagagcagcuuguacaggaucaauauggauguuauguaauccaacauguacuggagcacggucguccugaggauaaaagcaaaauuguagcagaaauccgaggcaauguacuuguauugagucagcacaaauuugcaugcaauguugugcagaaguguguuacucacgccucacguacggagcgcgcugugcucaucgaugaggugugcaccaugaacgacgguccccacagugccuuauacaccaugaugaaggaccaguaugccagcuacgugguccgcaagaugauugacguggcggagccaggccagcggaagaucgucaugcauaagauccgaccccacaucgcaacucuucguaaguacaccuauggcaagcacauucuggccaagcuggagaaguacuacaugaagaacgguguugacuuagggggagguggcggaucgggagguggcggaucgggagguggcggaucgggccgcagccgccuuuuggaagauuuucgaaacaaccgguaccccaauuuacaacugcgggagauugccggacauauaauggaauuuucccaagaccagcaugggaacagauucauucagcugaaacuggagcgugccacaccagcugagcgccagcuugucuuuaaugaaauccuccaggcugccuaccaacucaugguggauguguuugguuguuacgucauucagaaguucuuugaauuuggcagucuugaacagaagcuggcuuuggcagaacggauucgaggucacguccugucauuggcacuacagauguauggcucccguguuaucgagaaagcucuugaguuuauuccuucagaccagcagaaugagaugguucgggaacuagauggccaugucuugaagugugugaaagaucagaauggcaaucacgugguucagaaaugcauugaauguguacagccccagucuuugcaauuuaucaucgaugcguuuaagggacagguauuugccuuauccacacauccuuauggcugccgagugauucagagaauccuggagcacugucucccugaccagacacucccuauuuuagaggagcuucaccagcacacagagcagcuuguacaggaucaauauggaaguuauguaauccgccauguacuggagcacggucguccugaggauaaaagcaaaauuguagcagaaauccgaggcaauguacuuguauugagucagcacaaauuugcaaacaauguugugcagaaguguguuacucacgccucacguacggagcgcgcugugcucaucgaugaggugugcaccaugaacgacgguccccacagugccuuauacaccaugaugaaggaccaguaugccugcuacgugguccagaagaugauugacguggcggagccaggccagcggaagaucgucaugcauaagauccgaccccacaucgcaacucuucguaaguacaccuauggcaagcacauucuggccaagcuggagaaguacuacaugaagaacgguguugacuuagggagcggcggcaagcggcccgccgccaccaagaaggccggccaggccaagaagaagaaguga

SEQ ID NO: 9 - RNA, 2145 nt, Artificial Sequence
  source: /note=“Description of Artificial Sequence Synthetic polynucleotide”
augggccgcagccgccuuuuggaagauuuucgaaacaaccgguaccccaauuuacaacugcgggagauugccggacauauaauggaauuuucccaagaccagcauggguccagauucauucagcugaaacuggagcgugccacaccagcugagcgccagcuugucuuuaaugaaauccuccaggcugccuaccaacucaugguggauguguuugguuguuacgucauucagaaguucuuugaauuuggcagucuugaacagaagcuggcuuuggcagaacggauucgaggucacguccugucauuggcacuacagauguauggcucccguguuauccgcaaagcucuugaguuuauuccuucagaccagcagaaugagaugguucgggaacuagauggccaugucuugaagugugugaaagaucagaauggcugucacgugguucagaaaugcauugaauguguacagccccagucuuugcaauuuaucaucgaugcguuuaagggccagguauuugccuuauccacacauccuuauggcucccgagugauugagagaauccuggagcacugucucccugaccagacacucccuauuuuagaggagcuucaccagcacacagagcagcuuguacaggaucaauauggauguuauguaauccaacauguacuggagcacggucguccugaggauaaaagcaaaauuguagcagaaauccgaggcaauguacuuguauugagucagcacaaauuugcaaacaauguugugcagaaguguguuacucacgccucacguacggagcgcgcugugcucaucgaugaggugugcaccaugaacgacgguccccacagugccuuauacaccaugaugaaggaccaguaugccaacuacgugguccagaagaugauugacguggcggagccaggccagcggaagaucgucaugcauaagauccgaccccacaucgcaacucuucguaaguacaccuauggcaagcacauucuggccaagcuggagaaguacuacaugaagaacgguguugacuuagggggagguggcggaucgggagguggcggaucgggagguggcggaucgggccgcagccgccuuuuggaagauuuucgaaacaaccgguaccccaauuuacaacugcgggagauugccggacauauaauggaauuuucccaagaccagcaugggaacagauucauucagcugaaacuggagcgugccacaccagcugagcgccagcuugucuuuaaugaaauccuccaggcugccuaccaacucaugguggauguguuugguaauuacgucauucagaaguucuuugaauuuggcagucuugaacagaagcuggcuuuggcagaacggauucgaggucacguccugucauuggcacuacagauguauggcucccguguuaucgagaaagcucuugaguuuauuccuucagaccagcagaaugagaugguucgggaacuagauggccaugucuugaagugugugaaagaucagaauggcagucacgugguugagaaaugcauugaauguguacagccccagucuuugcaauuuaucaucgaugcguuuaagggacagguauuugccuuauccacacauccuuauggcucccgagugauugagagaauccuggagcacugucucccugaccagacacucccuauuuuagaggagcuucaccagcacacagagcagcuuguacaggaucaauauggauguuauguaauccaacauguacuggagcacggucguccugaggauaaaagcaaaauuguagcagaaauccgaggcaauguacuuguauugagucagcacaaauuugcaagcuauguugugcgcaaguguguuacucacgccucacguacggagcgcgcugugcucaucgaugaggugugcaccaugaacgacgguccccacagugccuuauacaccaugaugaaggaccaguaugccugcuacgugguccagaagaugauugacguggcggagccaggccagcggaagaucgucaugcauaagauccgaccccacaucgcaacucuucguaaguacaccuauggcaagcacauucuggccaagcuggagaaguacuacaugaagaacgguguugacuuaggguga

SEQ ID NO: 10 - RNA, 3573 nt, Artificial Sequence
  source: /note=“Description of Artificial Sequence Synthetic polynucleotide”
auggacuauaaggaccacgacggagacuacaaggaucaugauauugauuacaaagacgaugacgauaagauggccccaaagaagaagcggaaggucgguauccacggagucccagcagcccuccaccucgaccaaacacccagcagacagccuaucccuuccgaaggacugcagcugcauuuaccgcagguuuuagcugacgcugucucacgccugguccuggguaaguuuggugaucugaccgacaacuucuccuccccucacgcucgcagaaaagugcuggcuggagucgucaugacaacaggcacagauguuaaagaugccaaggugauaaguguuucuacaggaggcaaauguauuaauggugaauacaugagugaucguggccuugcauuaaaugacugccaugcagaaauaauaucucggagauccuugcucagauuucuuuauacacaacuugagcuuuacuuaaauaacaaagaugaucaaaaaagauccaucuuucagaaaucagagcgagggggguuuaggcugaaggagaauguccaguuucaucuguacaucagcaccucucccuguggagaugccagaaucuucucaccacaugagccaauccuggaagaaccagcagauagacacccaaaucguaaagcaagaggacagcuacggaccaaaauagagucuggucaggggacgauuccagugcgcuccaaugcgagcauccaaacgugggacggggugcugcaaggggagcggcugcucaccauguccugcagugacaagauugcacgcuggaacguggugggcauccagggaucacugcucagcauuuucguggagcccauuuacuucucgagcaucauccugggcagccuuuaccacggggaccaccuuuccagggccauguaccagcggaucuccaacauagaggaccugccaccucucuacacccucaacaagccuuugcucaguggcaucagcaaugcagaagcacggcagccagggaaggcccccaacuucagugucaacuggacgguaggcgacuccgcuauugaggucaucaacgccacgacugggaaggaugagcugggccgcgcgucccgccuguguaagcacgcguuguacugucgcuggaugcgugugcacggcaagguucccucccacuuacuacgcuccaagauuaccaagcccaacguguaccaugaguccaagcuggcggcaaaggaguaccaggccgccaaggcgcgucuguucacagccuucaucaaggcggggcugggggccuggguggagaagcccaccgagcaggaccaguucucacucacgccuggagguggcggaucgggagguggcggaucgggagguggcggaucgggccgcagccgccuuuuggaagauuuucgaaacaaccgguaccccaauuuacaacugcgggagauugccggacauauaauggaauuuucccaagaccagcauggguccagauucauucagcugaaacuggagcgugccacaccagcugagcgccagcuugucuuuaaugaaauccuccaggcugccuaccaacucaugguggauguguuugguuguuacgucauucagaaguucuuugaauuuggcagucuugaacagaagcuggcuuuggcagaacggauucgaggucacguccugucauuggcacuacagauguauggcucccguguuauccgcaaagcucuugaguuuauuccuucagaccagcagaaugagaugguucgggaacuagauggccaugucuugaagugugugaaagaucagaauggcugucacgugguucagaaaugcauugaauguguacagccccagucuuugcaauuuaucaucgaugcguuuaagggccagguauuugccuuauccacacauccuuauggcucccgagugauugagagaauccuggagcacugucucccugaccagacacucccuauuuuagaggagcuucaccagcacacagagcagcuuguacaggaucaauauggauguuauguaauccaacauguacuggagcacggucguccugaggauaaaagcaaaauuguagcagaaauccgaggcaauguacuuguauugagucagcacaaauuugcaaacaauguugugcagaaguguguuacucacgccucacguacggagcgcgcugugcucaucgaugaggugugcaccaugaacgacgguccccacagugccuuauacaccaugaugaaggaccaguaugccaacuacgugguccagaagaugauugacguggcggagccaggccagcggaagaucgucaugcauaagauccgaccccacaucgcaacucuucguaaguacaccuauggcaagcacauucuggccaagcuggagaaguacuacaugaagaacgguguugacuuagggggagguggcggaucgggagguggcggaucgggagguggcggaucgggccgcagccgccuuuuggaagauuuucgaaacaaccgguaccccaauuuacaacugcgggagauugccggacauauaauggaauuuucccaagaccagcaugggaacagauucauucagcugaaacuggagcgugccacaccagcugagcgccagcuugucuuuaaugaaauccuccaggcugccuaccaacucaugguggauguguuugguaauuacgucauucagaaguucuuugaauuuggcagucuugaacagaagcuggcuuuggcagaacggauucgaggucacguccugucauuggcacuacagauguauggcucccguguuaucgagaaagcucuugaguuuauuccuucagaccagcagaaugagaugguucgggaacuagauggccaugucuugaagugugugaaagaucagaauggcagucacgugguugagaaaugcauugaauguguacagccccagucuuugcaauuuaucaucgaugcguuuaagggacagguauuugccuuauccacacauccuuauggcucccgagugauugagagaauccuggagcacugucucccugaccagacacucccuauuuuagaggagcuucaccagcacacagagcagcuuguacaggaucaauauggauguuauguaauccaacauguacuggagcacggucguccugaggauaaaagcaaaauuguagcagaaauccgaggcaauguacuuguauugagucagcacaaauuugcaagcuauguugugcgcaaguguguuacucacgccucacguacggagcgcgcugugcucaucgaugaggugugcaccaugaacgacgguccccacagugccuuauacaccaugaugaaggaccaguaugccugcuacgugguccagaagaugauugacguggcggagccaggccagcggaagaucgucaugcauaagauccgaccccacaucgcaacucuucguaaguacaccuauggcaagcacauucuggccaagcuggagaaguacuacaugaagaacgguguugacuuagggagcggcggcaagcggcccgccgccaccaagaaggccggccaggccaagaagaagaaguga

SEQ ID NO: 11 - RNA, 828 nt, Artificial Sequence
  source: /note=“Description of Artificial Sequence Synthetic polynucleotide”
auggacuacaaggaccacgauggagauuauaaagaccacgacaucgacuauaaggacgacgacgacaagaugagcgacagcggcgagcagaacuacggcgagagagaguccagaagcgccagcagauccggcuccgcucacggaagcggaaagagcgcuagacauacccccgccagaagcagauccaaggaggauucuagaaggagcagaagcaagagcagaucuagaagcgaaucuagauccagaucuagaagaagcucuagaaggcacuacacaaggucuagaagcagaucuagaagccauagaagaagcagauccagaagcuacucuagagacuacagaaggagacacagccacucccacagcccuauguccacaagaagaaggcacgugggcaauagggccaaccccgacccuaaccccaagaagaagaggaaggugggcuccggcgucuucggcgaagacggcagcggcccuaagaagaagaggaaggugggcagcagcagcaucaccaagagaccccacaccccuacccccggcaucuacaugggcagacccaccuacggcuccucuagaaggagagacuacuacgacagaggcuacgauagaggcuacgacgauagagauuauuacucuagauccuacagaggcggcggaggaggcggaggcggauggagagcugcccaagacagagaccagaucuauagaagaaggagccccagccccuacuauagcagaggcggcuacagaucuagaucuagaucuagaagcuauagccccagaagauacggcggcagcuacccuuacgacgugcccgacuacgccuga

SEQ ID NO: 12 - RNA, 2964 nt, Artificial Sequence
  source: /note=“Description of Artificial Sequence Synthetic polynucleotide”
auggacuacaaggaccacgauggagauuauaaagaccacgacaucgacuauaaggacgacgacgacaagaugagcgacagcggcgagcagaacuacggcgagagagaguccagaagcgccagcagauccggcuccgcucacggaagcggaaagagcgcuagacauacccccgccagaagcagauccaaggaggauucuagaaggagcagaagcaagagcagaucuagaagcgaaucuagauccagaucuagaagaagcucuagaaggcacuacacaaggucuagaagcagaucuagaagccauagaagaagcagauccagaagcuacucuagagacuacagaaggagacacagccacucccacagcccuauguccacaagaagaaggcacgugggcaauagggccaaccccgacccuaaccccaagaagaagaggaaggugggcggagguggcggaucgggcaggagcaggcuuuuggaagauuuucgaaacaaccgcuaccccaauuuacaacugcgggagauugcuggacauauaauggaauuuucccaagaccagcauggguccagauucauucagcugaaacuggagcgugccacaccagcugagcgccagcuugucuucaaugaaauccuccaggcugccuaccaacucaugguggauguguuugguaauuacgucauucagaaguucuuugaauuuggcagucuugaacagaagcuggcuuuggcagaacggauucgaggccacguccugucauuggcacuacagauguauggcugccguguuauccagaaagcucuugaguuuauuccuucagaccagcagaaugagaugguucgggaacuagauggccaugucuugaagugugugaaagaucagaauggcaaucacgugguucagaaaugcauugaauguguacagccccagucuuugcaauuuaucaucgaugcguuuaagggacagguauuugccuuauccacacauccuuauggcugccgagugauucagagaauccuggagcacugucucccugaccagacacucccuauuuuagaggagcuucaccagcacacagagcagcuuguacaggaucaauauggaaauuauguaauccaacauguacuggagcacggucguccugaggauaaaagcaaaauuguagcagaaauccgaggcaauguacuuguauugagucagcacaaauuugcaagcaauguuguggagaaguguguuacucacgccucacguacggagcgcgcugugcucaucgaugaggugugcaccaugaacgacgguccccacagugccuuauacaccaugaugaaggaccaguaugccaacuacgugguccagaagaugauugacguggcggagccaggccagcggaagaucgucaugcauaagauccggccccacaucgcaacucuucguaaguacaccuauggcaagcacauucuggccaagcuggagaaguacuacaugaagaacgguguugacuuagggggagguggcggaucgggagguggcggaucgggagguggcggaucgggcaggagcaggcuuuuggaagauuuucgaaacaaccgcuaccccaauuuacaacugcgggagauugcuggacauauaauggaauuuucccaagaccagcauggguccagauucauucagcugaaacuggagcgugccacaccagcugagcgccagcuugucuucaaugaaauccuccaggcugccuaccaacucaugguggauguguuugguaauuacgucauucagaaguucuuugaauuuggcagucuugaacagaagcuggcuuuggcagaacggauucgaggccacguccugucauuggcacuacagauguauggcugccguguuauccagaaagcucuugaguuuauuccuucagaccagcagaaugagaugguucgggaacuagauggccaugucuugaagugugugaaagaucagaauggcaaucacgugguucagaaaugcauugaauguguacagccccagucuuugcaauuuaucaucgaugcguuuaagggacagguauuugccuuauccacacauccuuauggcugccgagugauucagagaauccuggagcacugucucccugaccagacacucccuauuuuagaggagcuucaccagcacacagagcagcuuguacaggaucaauauggaaauuauguaauccaacauguacuggagcacggucguccugaggauaaaagcaaaauuguagcagaaauccgaggcaauguacuuguauugagucagcacaaauuugcaagcaauguuguggagaaguguguuacucacgccucacguacggagcgcgcugugcucaucgaugaggugugcaccaugaacgacgguccccacagugccuuauacaccaugaugaaggaccaguaugccaacuacgugguccagaagaugauugacguggcggagccaggccagcggaagaucgucaugcauaagauccggccccacaucgcaacucuucguaaguacaccuauggcaagcacauucuggccaagcuggagaaguacuacaugaagaacgguguugacuuagggagcggcggcggcccuaagaagaagaggaaggugggcagcagcagcaucaccaagagaccccacaccccuacccccggcaucuacaugggcagacccaccuacggcuccucuagaaggagagacuacuacgacagaggcuacgauagaggcuacgacgauagagauuauuacucuagauccuacagaggcggcggaggaggcggaggcggauggagagcugcccaagacagagaccagaucuauagaagaaggagccccagccccuacuauagcagaggcggcuacagaucuagaucuagaucuagaagcuauagccccagaagauacggcggcagcuacccuuacgacgugcccgacuacgccuga

SEQ ID NO: 13 - protein, 714 aa, Artificial Sequence
  source: /note=“Description of Artificial Sequence Synthetic polypeptide”
MGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLGGGGGSGGGGSGGGGSGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLG

SEQ ID NO: 14 - protein, 404 aa, Artificial Sequence
  source: /note=“Description of Artificial Sequence Synthetic polypeptide”
MLHLDQTPSRQPIPSEGLQLHLPQVLADAVSRLVLGKFGDLTDNFSSPHARRKVLAGVVMTTGTDVKDAKVISVSTGGKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 15 - protein, 1190 aa, Artificial Sequence
  source: /note=“Description of Artificial Sequence Synthetic polypeptide”
MDYKDHDGDYKDHDIDYKDDDDKMAPKKKRKVGIHGVPAALHLDQTPSRQPIPSEGLQLHLPQVLADAVSRLVLGKFGDLTDNFSSPHARRKVLAGVVMTTGTDVKDAKVISVSTGGKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTPGGGGSGGGGSGGGGSGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLGGGGGSGGGGSGGGGSGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLGSGGKRPAATKKAGQAKKKK

SEQ ID NO: 16 - protein, 714 aa, Artificial Sequence
  source: /note=“Description of Artificial Sequence Synthetic polypeptide”
MGRSRLLEDFRNNRYPNLQLREIAGHIMEFSQDQHGSRFIRLKLERATPAERQLVFNEILQAAYQLMVDVFGSYVIEKFFEFGSLEQKLALAERIRGHVLSLALQMYGCRVIQKALEFIPSDQQNEMVRELDGHVLKCVKDQNGCHVVQKCIECVQPQSLQFIIDAFKGQVFALSTHPYGSRVIERILEHCLPDQTLPILEELHQHTEQLVQDQYGCYVIQHVLEHGRPEDKSKIVAEIRGNVLVLSQHKFACNVVQKCVTHASRTERAVLIDEVCTMNDGPHSALYTMMKDQYASYVVRKMIDVAEPGQRKIVMHKIRPHIATLRKYTYGKHILAKLEKYYMKNGVDLGGGGGSGGGGSGGGGSGRSRLLEDFRNNRYPNLQLREIAGHIMEFSQDQHGNRFIQLKLERATPAERQLVFNEILQAAYQLMVDVFGCYVIQKFFEFGSLEQKLALAERIRGHVLSLALQMYGSRVIEKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGSYVIRHVLEHGRPEDKSKIVAEIRGNVLVLSQHKFANNVVQKCVTHASRTERAVLIDEVCTMNDGPHSALYTMMKDQYACYVVQKMIDVAEPGQRKIVMHKIRPHIATLRKYTYGKHILAKLEKYYMKNGVDLG

SEQ ID NO: 17 - protein, 1190 aa, Artificial Sequence
  source: /note=“Description of Artificial Sequence Synthetic polypeptide”
MDYKDHDGDYKDHDIDYKDDDDKMAPKKKRKVGIHGVPAALHLDQTPSRQPIPSEGLQLHLPQVLADAVSRLVLGKFGDLTDNFSSPHARRKVLAGVVMTTGTDVKDAKVISVSTGGKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTPGGGGSGGGGSGGGGSGRSRLLEDFRNNRYPNLQLREIAGHIMEFSQDQHGSRFIRLKLERATPAERQLVFNEILQAAYQLMVDVFGSYVIEKFFEFGSLEQKLALAERIRGHVLSLALQMYGCRVIQKALEFIPSDQQNEMVRELDGHVLKCVKDQNGCHVVQKCIECVQPQSLQFIIDAFKGQVFALSTHPYGSRVIERILEHCLPDQTLPILEELHQHTEQLVQDQYGCYVIQHVLEHGRPEDKSKIVAEIRGNVLVLSQHKFACNVVQKCVTHASRTERAVLIDEVCTMNDGPHSALYTMMKDQYASYVVRKMIDVAEPGQRKIVMHKIRPHIATLRKYTYGKHILAKLEKYYMKNGVDLGGGGGSGGGGSGGGGSGRSRLLEDFRNNRYPNLQLREIAGHIMEFSQDQHGNRFIQLKLERATPAERQLVFNEILQAAYQLMVDVFGCYVIQKFFEFGSLEQKLALAERIRGHVLSLALQMYGSRVIEKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGSYVIRHVLEHGRPEDKSKIVAEIRGNVLVLSQHKFANNVVQKCVTHASRTERAVLIDEVCTMNDGPHSALYTMMKDQYACYVVQKMIDVAEPGQRKIVMHKIRPHIATLRKYTYGKHILAKLEKYYMKNGVDLGSGGKRPAATKKAGQAKKKK

SEQ ID NO: 18 - protein, 714 aa, Artificial Sequence
  source: /note=“Description of Artificial Sequence Synthetic polypeptide”
MGRSRLLEDFRNNRYPNLQLREIAGHIMEFSQDQHGSRFIQLKLERATPAERQLVFNEILQAAYQLMVDVFGCYVIQKFFEFGSLEQKLALAERIRGHVLSLALQMYGSRVIRKALEFIPSDQQNEMVRELDGHVLKCVKDQNGCHVVQKCIECVQPQSLQFIIDAFKGQVFALSTHPYGSRVIERILEHCLPDQTLPILEELHQHTEQLVQDQYGCYVIQHVLEHGRPEDKSKIVAEIRGNVLVLSQHKFANNVVQKCVTHASRTERAVLIDEVCTMNDGPHSALYTMMKDQYANYVVQKMIDVAEPGQRKIVMHKIRPHIATLRKYTYGKHILAKLEKYYMKNGVDLGGGGGSGGGGSGGGGSGRSRLLEDFRNNRYPNLQLREIAGHIMEFSQDQHGNRFIQLKLERATPAERQLVFNEILQAAYQLMVDVFGNYVIQKFFEFGSLEQKLALAERIRGHVLSLALQMYGSRVIEKALEFIPSDQQNEMVRELDGHVLKCVKDQNGSHVVEKCIECVQPQSLQFIIDAFKGQVFALSTHPYGSRVIERILEHCLPDQTLPILEELHQHTEQLVQDQYGCYVIQHVLEHGRPEDKSKIVAEIRGNVLVLSQHKFASYVVRKCVTHASRTERAVLIDEVCTMNDGPHSALYTMMKDQYACYVVQKMIDVAEPGQRKIVMHKIRPHIATLRKYTYGKHILAKLEKYYMKNGVDLG

SEQ ID NO: 19 - protein, 1190 aa, Artificial Sequence
  source: /note=“Description of Artificial Sequence Synthetic polypeptide”
MDYKDHDGDYKDHDIDYKDDDDKMAPKKKRKVGIHGVPAALHLDQTPSRQPIPSEGLQLHLPQVLADAVSRLVLGKFGDLTDNFSSPHARRKVLAGVVMTTGTDVKDAKVISVSTGGKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTPGGGGSGGGGSGGGGSGRSRLLEDFRNNRYPNLQLREIAGHIMEFSQDQHGSRFIQLKLERATPAERQLVFNEILQAAYQLMVDVFGCYVIQKFFEFGSLEQKLALAERIRGHVLSLALQMYGSRVIRKALEFIPSDQQNEMVRELDGHVLKCVKDQNGCHVVQKCIECVQPQSLQFIIDAFKGQVFALSTHPYGSRVIERILEHCLPDQTLPILEELHQHTEQLVQDQYGCYVIQHVLEHGRPEDKSKIVAEIRGNVLVLSQHKFANNVVQKCVTHASRTERAVLIDEVCTMNDGPHSALYTMMKDQYANYVVQKMIDVAEPGQRKIVMHKIRPHIATLRKYTYGKHILAKLEKYYMKNGVDLGGGGGSGGGGSGGGGSGRSRLLEDFRNNRYPNLQLREIAGHIMEFSQDQHGNRFIQLKLERATPAERQLVFNEILQAAYQLMVDVFGNYVIQKFFEFGSLEQKLALAERIRGHVLSLALQMYGSRVIEKALEFIPSDQQNEMVRELDGHVLKCVKDQNGSHVVEKCIECVQPQSLQFIIDAFKGQVFALSTHPYGSRVIERILEHCLPDQTLPILEELHQHTEQLVQDQYGCYVIQHVLEHGRPEDKSKIVAEIRGNVLVLSQHKFASYVVRKCVTHASRTERAVLIDEVCTMNDGPHSALYTMMKDQYACYVVQKMIDVAEPGQRKIVMHKIRPHIATLRKYTYGKHILAKLEKYYMKNGVDLGSGGKRPAATKKAGQAKKKK

SEQ ID NO: 20 - protein, 275 aa, Artificial Sequence
  source: /note=“Description of Artificial Sequence Synthetic polypeptide”
MDYKDHDGDYKDHDIDYKDDDDKMSDSGEQNYGERESRSASRSGSAHGSGKSARHTPARSRSKEDSRRSRSKSRSRSESRSRSRRSSRRHYTRSRSRSRSHRRSRSRSYSRDYRRRHSHSHSPMSTRRRHVGNRANPDPNPKKKRKVGSGVFGEDGSGPKKKRKVGSSSITKRPHTPTPGIYMGRPTYGSSRRRDYYDRGYDRGYDDRDYYSRSYRGGGGGGGGWRAAQDRDQIYRRRSPSPYYSRGGYRSRSRSRSYSPRRYGGSYPYDVPDYA

SEQ ID NO: 21 - protein, 987 aa, Artificial Sequence
  source: /note=“Description of Artificial Sequence Synthetic polypeptide”
MDYKDHDGDYKDHDIDYKDDDDKMSDSGEQNYGERESRSASRSGSAHGSGKSARHTPARSRSKEDSRRSRSKSRSRSESRSRSRRSSRRHYTRSRSRSRSHRRSRSRSYSRDYRRRHSHSHSPMSTRRRHVGNRANPDPNPKKKRKVGGGGGSGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLGGGGGSGGGGSGGGGSGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLGSGGGPKKKRKVGSSSITKRPHTPTPGIYMGRPTYGSSRRRDYYDRGYDRGYDDRDYYSRSYRGGGGGGGGWRAAQDRDQIYRRRSPSPYYSRGGYRSRSRSRSYSPRRYGGSYPYDVPDYA

SEQ ID NO: 22 - RNA, 16 nt, Homo sapiens
aucaugaucaagaagc

SEQ ID NO: 23 - RNA, 16 nt, Homo sapiens
acaggguuuuagacaa

SEQ ID NO: 24 - RNA, 16 nt, Homo sapiens
acaggguuuuagacaa

SEQ ID NO: 25 - RNA, 70 nt, Artificial Sequence
  source: /note=“Description of Artificial Sequence Synthetic oligonucleotide”
ggcuauggcaucgcaacaccuaaaggauccucauuaagguggguggaauaguauaacaauaugcuaaaug

SEQ ID NO: 26 - RNA, 66 nt, Artificial Sequence
  source: /note=“Description of Artificial Sequence Synthetic oligonucleotide”
uuuuuuaacuuccuuuauuuuccuuacaggguuuuagacaaaaucaaaaagaaggaaggugcucac

SEQ ID NO: 27 - DNA, 30 bp, Artificial Sequence
  source: /note=“Description of Artificial Sequence Synthetic oligonucleotide”
actattccacccaccgtaatgaggatcctt

SEQ ID NO: 28 - DNA, 18 bp, Artificial Sequence
  source: /note=“Description of Artificial Sequence Synthetic oligonucleotide”
tcactttcataatgctgg

SEQ ID NO: 29 - protein, 11 aa, Artificial Sequence
  source: /note=“Description of Artificial Sequence Synthetic peptide”
QYGGYVIRHVL

SEQ ID NO: 30 - protein, 351 aa, Artificial Sequence
  source: /note=“Description of Artificial Sequence Synthetic polypeptide”
GRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQVINEMVRELDGHVLKCVKDQNGNHVVQKCIECVQPQSLQFIIDAFKGQVFALSTHPYGCRVIQRILEHCLPDQTLPILEELHQHTEQLVQDQYGGYVIRHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLG

SEQ ID NO: 31 - protein, 36 aa, Homo sapiens
HIMEFSQDQHGSRFIQLKLERATPAERQLVFNEILQ

SEQ ID NO: 32 - protein, 36 aa, Homo sapiens
HVLSLALQMYGCRVIQKALEFIPSDQQNEMVRELDG

SEQ ID NO: 33 - protein, 36 aa, Homo sapiens
QVFALSTHPYGCRVIQRILEHCLPDQTLPILEELHQ

SEQ ID NO: 34 - protein, 43 aa, Homo sapiens
NVLVLSQHKFASNVVEKCVTHASRTERAVLIDEVCTMNDGPHS

SEQ ID NO: 35 - protein, 36 aa, Homo sapiens
AAYQLMVDVFGNYVIQKFFEFGSLEQKLALAERIRG

SEQ ID NO: 36 - protein, 36 aa, Homo sapiens
HVLKCVKDQNGNHVVQKCIECVQPQSLQFIIDAFKG

SEQ ID NO: 37 - protein, 36 aa, Homo sapiens
HTEQLVQDQYGNYVIQHVLEHGRPEDKSKIVAEIRG

SEQ ID NO: 38 - protein, 66 aa, Homo sapiens
ALYTMMKDQYANYVVQKMIDVAEPGQRKIVMHKIRPHIATLRKYTYGKHILAKLEKYYMKNGVDLG

SEQ ID NO: 39 - protein, 11 aa, Artificial Sequence
  source: /note=“Description of Artificial Sequence Synthetic peptide”
  VARIANT (4)..(4): /replace=“Ala” or “Ser” or “Thr” or “Cys”
  SITE (1)..(11): /note=“Variant residues given in the sequence have no preference with respect to those in the annotations for variant positions”
QHGGRFIRLKL

SEQ ID NO: 40 - protein, 11 aa, Artificial Sequence
  source: /note=“Description of Artificial Sequence Synthetic peptide”
  VARIANT (4)..(4): /replace=“Ala” or “Ser” or “Thr” or “Cys”
  SITE (1)..(11): /note=“Variant residues given in the sequence have no preference with respect to those in the annotations for variant positions”
VFGGYVIRKFF

SEQ ID NO: 41 - protein, 11 aa, Artificial Sequence
  source: /note=“Description of Artificial Sequence Synthetic peptide”
  VARIANT (4)..(4): /replace=“Ala” or “Ser” or “Thr” or “Cys”
  SITE (1)..(11): /note=“Variant residues given in the sequence have no preference with respect to those in the annotations for variant positions”
MYGGRVIRKAL

SEQ ID NO: 42 - protein, 11 aa, Artificial Sequence
  source: /note=“Description of Artificial Sequence Synthetic peptide”
  VARIANT (4)..(4): /replace=“Ala” or “Ser” or “Thr” or “Cys”
  SITE (1)..(11): /note=“Variant residues given in the sequence have no preference with respect to those in the annotations for variant positions”
QNGGHVVRKCI

SEQ ID NO: 43 - protein, 11 aa, Artificial Sequence
  source: /note=“Description of Artificial Sequence Synthetic peptide”
  VARIANT (4)..(4): /replace=“Ala” or “Ser” or “Thr” or “Cys”
  SITE (1)..(11): /note=“Variant residues given in the sequence have no preference with respect to those in the annotations for variant positions”
PYGGRVIRRIL

SEQ ID NO: 44 - protein, 11 aa, Artificial Sequence
  source: /note=“Description of Artificial Sequence Synthetic peptide”
  VARIANT (4)..(4): /replace=“Ala” or “Ser” or “Thr” or “Cys”
  SITE (1)..(11): /note=“Variant residues given in the sequence have no preference with respect to those in the annotations for variant positions”
QYGGYVIRHVL

SEQ ID NO: 45 - protein, 11 aa, Artificial Sequence
  source: /note=“Description of Artificial Sequence Synthetic peptide”
  VARIANT (4)..(4): /replace=“Ala” or “Ser” or “Thr” or “Cys”
  SITE (1)..(11): /note=“Variant residues given in the sequence have no preference with respect to those in the annotations for variant positions”
KFAGNVVRKCV

SEQ ID NO: 46 - protein, 11 aa, Artificial Sequence
  source: /note=“Description of Artificial Sequence Synthetic peptide”
  VARIANT (4)..(4): /replace=“Ala” or “Ser” or “Thr” or “Cys”
  SITE (1)..(11): /note=“Variant residues given in the sequence have no preference with respect to those in the annotations for variant positions”
QYAGYVVRKMI

SEQ ID NO: 47 - protein, 15 aa, Artificial Sequence
  source: /note=“Description of Artificial Sequence Synthetic peptide”
GGGGSGGGGSGGGGS

SEQ ID NO: 48 - protein, 34 aa, Mus musculus
SNHVEVNFLEKFTTERYFRPTWFLSWSPCGECSR

SEQ ID NO: 49 - protein, 34 aa, Homo sapiens
TNHVEVNFIKKFTSERDFHPTWFLSWSPCWECSQ

SEQ ID NO: 50 - protein, 34 aa, Oryctolagus cuniculus
TNHVEVNFLEKLTSEGRLGPTWFLSWSPCWECSM

SEQ ID NO: 51 - protein, 32 aa, Homo sapiens
AAHAEEAFFNTILPAFDPTWYVSSSPCAACAD

SEQ ID NO: 52 - protein, 33 aa, Mus musculus
GCHVELLFLRYISDWDLDPTWFTSWSPCYDCAR

SEQ ID NO: 53 - protein, 34 aa, Homo sapiens
DCHAEIISRRGFIRFLYSELHLYISTAPCGDGAL

SEQ ID NO: 54 - protein, 34 aa, Xenopus laevis
DCHAEVVSRRGFIRFLYSQLHLYISTAPCGDGAL

SEQ ID NO: 55 - protein, 34 aa, Homo sapiens
DCHAEIISRRSLLRFLYTQLHLYISTSPCGDARI

SEQ ID NO: 56 - protein, 34 aa, Rattus sp.
DCHAEIISRRSLLRFLYAQLHLYISTSPCGDARI

SEQ ID NO: 57 - protein, 34 aa, Rattus sp.
DCHAEIVARRAFLHFLYTQLHLYVSTSPCGDARL

SEQ ID NO: 58 - protein, 34 aa, Caenorhabditis elegans
DCHAEILARRGLLRFLYSEVHLFINTAPCGVARI

SEQ ID NO: 59 - protein, 34 aa, Drosophila melanogaster
DSHAEIVSRRCLLKYLYAQFHLYINTAPCGDARI

SEQ ID NO: 60 - protein, 34 aa, Homo sapiens
VCHAELNAIMNKNSTDVKGCSMYVALFPCNECAK

SEQ ID NO: 61 - protein, 35 aa, Saccharomyces cerevisiae
CLHAEENALLEAGRDRVGQNATLYCDTCPCLTCSV

SEQ ID NO: 62 - protein, 34 aa, Homo sapiens
GICAERTAIQKAVSEGYKDFMQDDFISPCGACRQ

SEQ ID NO: 63 - protein, 34 aa, Escherichia coli
TVHAEQSAISHAWLSGEKALAITVNYTPCGHCRQ

SEQ ID NO: 64 - protein, 34 aa, Caenorhabditis elegans
VVHAEMNAIINKRCTTLHDCTVYVTLFPCNKCAQ

SEQ ID NO: 65 - protein, 34 aa, Escherichia coli
TAHAEIMALRQGGLVMQNYRTLYVTLEPCVMCAG

SEQ ID NO: 66 - protein, 34 aa, Haemophilus influenzae
TAHAEIIALRNGAKNIQNYRTLYVTLEPCTMCAG

SEQ ID NO: 67 - protein, 34 aa, Homo sapiens
DSHAEVIARRSFQRYLLHQLVFFSSHTPCGDASI

SEQ ID NO: 68 - protein, 34 aa, Saccharomyces cerevisiae
DCHAEILALRGANTVLLNRIALYISRLPCGDASM

SEQ ID NO: 69 - protein, 34 aa, Drosophila melanogaster
DSHAEVLARRGFLRFLYQELHFLSTQTPCGDACI

SEQ ID NO: 70 - protein, 35 aa, Homo sapiens
TRHAEMVAIDQVLDWCRQSGTVLYVTVEPCIMCAA

SEQ ID NO: 71 - protein, 34 aa, Saccharomyces cerevisiae
VAHAEFMGIDQIKAMLGSRGTLYVTVEPCIMCAS

SEQ ID NO: 72 - protein, 35 aa, Homo sapiens
LLHAVMVCVDLVARGQGRGGYDLYVTREPCAMCAM

SEQ ID NO: 73 - protein, 34 aa, Saccharomyces cerevisiae
IDHSVMVGIRAVGERLREGVDVYLTHEPCSMCSM

SEQ ID NO: 74 - protein, 5 aa, Unknown
  source: /note=“Description of Unknown Cleavable linker sequence”
CPRSC

SEQ ID NO: 75 - protein, 36 aa, Artificial Sequence
  source: /note=“Description of Artificial Sequence Synthetic polypeptide”
HIMEFSQDQHGCRFIRLKLERATPAERQLVFNEILQ

SEQ ID NO: 76 - protein, 36 aa, Artificial Sequence
  source: /note=“Description of Artificial Sequence Synthetic polypeptide”
AAYQLMVDVFGSRVIQKFFEFGSLEQKLALAERIRG

SEQ ID NO: 77 - protein, 36 aa, Artificial Sequence
  source: /note=“Description of Artificial Sequence Synthetic polypeptide”
AAYQLMVDVFGSNVIEKFFEFGSLEQKLALAERIRG

SEQ ID NO: 78 - protein, 36 aa, Artificial Sequence
  source: /note=“Description of Artificial Sequence Synthetic polypeptide”
HVLKCVKDQNGSRVVQKCIECVQPQSLQFIIDAFKG

SEQ ID NO: 79 - protein, 36 aa, Artificial Sequence; identical to SEQ ID NO: 33
  source: /note=“Description of Artificial Sequence Synthetic polypeptide”

SEQ ID NO: 80 - protein, 36 aa, Artificial Sequence
  source: /note=“Description of Artificial Sequence Synthetic polypeptide”
HTEQLVQDQYGSNVIEHVLEHGRPEDKSKIVAEIRG

SEQ ID NO: 81 - protein, 43 aa, Artificial Sequence; identical to SEQ ID NO: 34
  source: /note=“Description of Artificial Sequence Synthetic polypeptide”

SEQ ID NO: 82 - protein, 66 aa, Artificial Sequence
  source: /note=“Description of Artificial Sequence Synthetic polypeptide”
ALYTMMKDQYACYVVRKMIDVAEPGQRKIVMHKIRPHIATLRKYTYGKHILAKLEKYYMKNGVDLG

SEQ ID NO: 83 - DNA, 20 bp, Artificial Sequence
  source: /note=“Description of Artificial Sequence Synthetic primer”
ccatcgaaagtgctgaggat

SEQ ID NO: 84 - DNA, 20 bp, Artificial Sequence
  source: /note=“Description of Artificial Sequence Synthetic primer”
agggctctgcactcctcata

SEQ ID NO: 85 - protein, 36 aa, Artificial Sequence
  source: /note=“Description of Artificial Sequence Synthetic polypeptide”
HIMEFSQDQHGNYFIQLKLERATPAERQLVFNEILQ

SEQ ID NO: 86 - protein, 36 aa, Artificial Sequence; identical to SEQ ID NO: 35
  source: /note=“Description of Artificial Sequence Synthetic polypeptide”

SEQ ID NO: 87 - protein, 36 aa, Artificial Sequence
  source: /note=“Description of Artificial Sequence Synthetic polypeptide”
AAYQLMVDVFGCRVIQKFFEFGSLEQKLALAERIRG

SEQ ID NO: 88 - protein, 36 aa, Artificial Sequence
  source: /note=“Description of Artificial Sequence Synthetic polypeptide”
HVLKCVKDQNGSNVVEKCIECVQPQSLQFIIDAFKG

SEQ ID NO: 89 - protein, 36 aa, Artificial Sequence
  source: /note=“Description of Artificial Sequence Synthetic polypeptide”
HTEQLVQDQYGCYVIRHVLEHGRPEDKSKIVAEIRG

SEQ ID NO: 90 - protein, 43 aa, Artificial Sequence
  source: /note=“Description of Artificial Sequence Synthetic polypeptide”
NVLVLSQHKFACRVVQKCVTHASRTERAVLIDEVCTMNDGPHS

SEQ ID NO: 91 - protein, 66 aa, Artificial Sequence
  source: /note=“Description of Artificial Sequence Synthetic polypeptide”
ALYTMMKDQYACRVVQKMIDVAEPGQRKIVMHKIRPHIATLRKYTYGKHILAKLEKYYMKNGVDLG

SEQ ID NO: 92 - DNA, 105 bp, Unknown
  source: /note=“Description of Unknown EBNA1 GAr sequence”
taaaggagcaggagcaggagcgggaggggcaggagcaggaggggcaggagcaggaggaggggcaggagcaggaggaggggcaggaggggcaggaggggcaggaat

SEQ ID NO: 93 - protein, 36 aa, Artificial Sequence
  source: /note=“Description of Artificial Sequence Synthetic polypeptide”
HIMEFSQDQHGSNFIELKLERATPAERQLVFNEILQ

SEQ ID NO: 94 - protein, 36 aa, Artificial Sequence
  source: /note=“Description of Artificial Sequence Synthetic polypeptide”
AAYQLMVDVFGCYVIRKFFEFGSLEQKLALAERIRG

SEQ ID NO: 95 - protein, 36 aa, Artificial Sequence
  source: /note=“Description of Artificial Sequence Synthetic polypeptide”
QVFALSTHPYGSNVIERILEHCLPDQTLPILEELHQ

SEQ ID NO: 96 - protein, 36 aa, Artificial Sequence
  source: /note=“Description of Artificial Sequence Synthetic polypeptide”
HTEQLVQDQYGSRVIQHVLEHGRPEDKSKIVAEIRG

SEQ ID NO: 97 - protein, 66 aa, Artificial Sequence
  source: /note=“Description of Artificial Sequence Synthetic polypeptide”
ALYTMMKDQYASNVVEKMIDVAEPGQRKIVMHKIRPHIATLRKYTYGKHILAKLEKYYMKNGVDLG

SEQ ID NO: 98 - DNA, 18 bp, Artificial Sequence
  source: /note=“Description of Artificial Sequence Synthetic primer”
tttttttttttttttttt

SEQ ID NO: 99 - DNA, 30 bp, Artificial Sequence
  source: /note=“Description of Artificial Sequence Synthetic oligonucleotide”
gggatatctatcatgatcaagaagcctcag

SEQ ID NO: 100 - DNA, 30 bp, Artificial Sequence
  source: /note=“Description of Artificial Sequence Synthetic oligonucleotide”
  modified_base (29)..(29): Inosine
gggatatctatcatgatcaagaagcctcng

SEQ ID NO: 101 - DNA, 34 bp, Unknown
  source: /note=“Description of Unknown EBNA1 sequence”
gccccccacggggacccacggaggaggggagggc

SEQ ID NO: 102 - RNA, 34 nt, Artificial Sequence
  Description of Artificial Sequence Synthetic oligonucleotide
gccccccacggggacccacggaggaggggagggc

SEQ ID NO: 103 - RNA, 32 nt, Artificial Sequence
  Description of Artificial Sequence Synthetic oligonucleotide
  modified_base (11)..(11): Inosine
  modified_base (14)..(14): Inosine
gcccgacgagncgnggccccgcgaggucgggc

(19) United States
(12) Patent Application Publication
(10) Pub. No.: US 2022/0024999 A1
(43) Pub. Date: Jan. 27, 2022

(54) METHODS OF MODULATING RNA

(71) Applicant: FLAGSHIP PIONEERING INNOVATIONS V, INC., Cambridge, MA (US)

(72) Inventors: David Arthur Berry, Newton, MA (US); Christalyn Rhodes, Dedham, MA (US); Jeremiah Dale Farelli, Marblehead, MA (US)

(21) Appl. No.: 17/298,317

(22) PCT Filed: Nov. 27, 2019
(86) PCT No.: PCT/US2019/063798

§ 371 Date: May 28, 2021

Related U.S. Application Data
(60) Provisional application No. 62/780,442, filed on Dec. 17, 2018.
(60) Provisional application No. 62/778,361, filed on Dec. 12, 2018.
(60) Provisional application No. 62/772,907, filed on Nov. 29, 2018.

(51) Int. Cl. C07K 14/47; C12N 15/113; A61K 31/7088; C12N 9/78; A61K 48/00; A61P 31/20; A61P 35/00; A61P 21/00

(52) U.S. Cl. CPC C07K 14/47; C12N 15/113; A61K 31/7088; C12N 9/78; A61K 48/0066; C07K 2319/85; A61P 35/00; A61P 21/00; C12N 2310/11; C12N 2320/31; A61P 31/20

(57) ABSTRACT

The present disclosure relates generally to methods and compositions for modulating RNA, e.g., using polypeptides comprising Pumilio homology domains.

### RELATED APPLICATIONS

This application claims priority to U.S. Ser. No. 62/772,907 filed Nov. 29, 2018, U.S. Ser. No. 62/778,361 filed Dec. 12, 2018, and U.S. Ser. No. 62/780,442 filed Dec. 17, 2018, the contents of which are each incorporated herein by reference in their entireties.

### SUMMARY

Described herein are compositions and methods for altering RNA structure and function to modulate biological processes.

The primary nucleotide sequence determines the secondary and tertiary structure of RNA. The base pairing of nucleotides forms stems, loops and combinations necessary for binding of RNA ligands such as proteins. As such, editing of the primary sequence and thereby the secondary and/or tertiary structure of an RNA can alter its ligand binding properties and provide a way of modulating downstream processes without altering the function of the ligand (e.g., an RNA-binding polypeptide). Described herein are compositions and related methods to modulate RNA primary, secondary, and tertiary structure and function, and/or splicing, to affect processes effected by RNA-ligand interactions and/or expression of the RNA encoded product.

Accordingly, in one aspect, the disclosure is directed to a polypeptide comprising: (a) an RNA binding domain comprising a plurality of (e.g., 2-50, 10-30, or 16-21) RNA base-binding motifs, each of which binds to an RNA base, and which are ordered in the RNA binding domain to bind to the consecutive order of the RNA bases in the target RNA sequence, linked to (b) a heterologous RNA editing domain.

In another aspect, the disclosure is directed to a polypeptide comprising: (a) an RNA binding domain comprising a plurality of (e.g., 2-50, 10-30, or 16-21) RNA base-binding motifs, each of which binds to an RNA base, and which are ordered in the RNA binding domain to bind to the consecutive order of the RNA bases in the target RNA sequence, linked to (b) a heterologous RNA editing domain, wherein the polypeptide does not comprise a nuclease or a functional fragment thereof.

In another aspect, the disclosure is directed to a polypeptide comprising: (a) an RNA binding domain comprising a plurality of (e.g., 2-50, 10-30, or 16-21) RNA base-binding motifs, each of which binds to an RNA base, and which are ordered in the RNA binding domain to bind to the consecutive order of the RNA bases in the target RNA sequence, linked to (b) a heterologous RNA editing domain comprising a catalytic domain of a deaminase or functional fragment or variant thereof.

In another aspect, the disclosure is directed to a polypeptide comprising: (a) an RNA binding domain comprising a plurality of (e.g., 2-50, 10-30, or 16-21) RNA base-binding motifs, each of which binds to an RNA base, and which are ordered in the RNA binding domain to bind to the consecutive order of the RNA bases in the target RNA sequence, linked to (b) a heterologous RNA effector comprising a splicing factor.

In some embodiments, the plurality of RNA base-binding motifs comprises at least 3 (e.g., at least 4 at least 5, at least 6, at least 7, at least 8, at least 9, between 14-24, between 15-23, between 16-22, between 16-21, between 2-20, between 2-15, between 2-10, between 2-8, between 3-20, between 3-15, between 3-10, between 3-8, between 4-8, up to 25, up to 30) PUM RNA-binding motifs.

In some embodiments, the RNA binding domain binds an RNA sequence of between 2-50 nucleotides (e.g., between 14-30, 15-26, 16-21, 2-40, 2-30, 2-25, 2-20, 5-50, 5-40, 5-30, 5-25, 5-20, 5-15, 2-18, 2-15, 2-12, 2-10, 2-9, 2-8, 3-20, 3-15, 3-10, 3-9, 3-8, 4-12, 4-10, 4-9, 4-8, 5-10, 5-9, 5-8 nucleotides).

In some embodiments, the RNA binding domain is between 90-500 amino acid residues, e.g., between 90-450 amino acid residues, between 90-400 amino acid residues, between 90-350 amino acid residues, between 90-300 amino acid residues, between 120-400 amino acid residues.

In some embodiments, the RNA binding domain has at least 80% identity (e.g., at least 85% identity, at least 87% identity, at least 90% identity, at least 92% identity, at least 95% identity, at least 97% identity, at least 98% identity, or 99% identity) and less than 100% identity to a corresponding amino acid sequence of a wild type PUM-HD, e.g., wild type human PUM1-HD.

In some embodiments, the RNA binding domain binds an RNA sequence comprising a disease-associated mutation.

In some embodiments, the RNA binding domain binds an RNA sequence comprising a disease-associated mutation and the RNA editing domain edits (e.g., corrects) the disease-associated mutation.

In some embodiments, the RNA editing domain comprises a polypeptide comprising a catalytic domain of an RNA deaminase (e.g., an adenosine deaminase or a cytidine deaminase) or a functional fragment or variant thereof.

In some embodiments, the RNA editing domain comprises the catalytic domain of an Adenosine Deaminase Acting on RNA (ADAR) (e.g., human ADAR 1, human ADAR2, human ADAR3, or human ADAR4); an Adenosine Deaminase Acting on tRNAs (ADAT); a Cytosine Deaminase Acting on RNA (CDAR); or a functional fragment or variant thereof.

In some embodiments, the catalytic domain of the deaminase is at least 80% identical (e.g., at least 85%, 87%, 90%, 92%, 95%, 98%, 99%, 100% identical) to a sequence shown in Table B.

In some embodiments, the RNA editing domain modifies at least 1, 2, 3, 4, 5, 6, 7, 8, 9, or 10 (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6- 7, 7-10, 7-9, 7-8, 8-10, 8-9, or 9-10) nucleotides of the target RNA sequence or an RNA comprising the target sequence.

In some embodiments, the RNA editing domain modifies a single nucleotide of the target RNA sequence or an RNA comprising the target sequence.

In some embodiments, the RNA editing domain changes a base to another base, e.g., changes a cytosine to a uracil; an adenosine to an inosine; or a guanosine to an adenosine.

In some embodiments, the RNA editing domain modifies an amino-acid encoding sequence of the target RNA sequence.

In some embodiments, the modification to the amino-acid encoding sequence of the target RNA sequence alters the amino acid sequence of a product polypeptide encoded by the target RNA sequence.

In some embodiments, the RNA editing domain modifies at least 1, 2, 3, 4, 5, 6, 7, 8, 9, or 10 (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6- 7, 7-10, 7-9, 7-8, 8-10, 8-9, or 9-10) nucleotides of the target RNA sequence, and optionally no more than 1, 2, 3, 4, 5, 6, 7, 8, 9, or 10 nucleotides of the target RNA sequence.

In some embodiments, the RNA binding domain binds a secondary structure of an RNA.

In some embodiments, the RNA binding domain binds a pre-mRNA, e.g., an intron-exon junction of a pre-mRNA.

In some embodiments, the polypeptide inhibits (e.g., formation of), destabilizes, and/or eliminates a secondary structure of the target RNA sequence or an RNA comprising the target RNA sequence.

In some embodiments, the polypeptide alters the splicing of the target RNA sequence or an RNA comprising the target RNA sequence.

In some embodiments, the polypeptide inhibits, e.g., eliminates, splicing of the target RNA sequence or an RNA comprising the target RNA sequence at a splice site (e.g., a target splice site), and optionally does not inhibit splicing of the target RNA sequence or an RNA comprising the target RNA sequence at one or more other splice site(s) (e.g., one or more non-target splice site(s)).

In some embodiments, the polypeptide decreases expression of a gene, e.g., a gene encoding the target RNA sequence.

In some embodiments, the polypeptide decreases the level of a product polypeptide encoded by the target RNA sequence.

In some embodiments, the polypeptide eliminates a stop codon, e.g., a premature stop codon, in the target RNA sequence or an RNA comprising the target RNA sequence.

In some embodiments, the polypeptide creates a stop codon, e.g., a premature stop codon, in the target RNA sequence or an RNA comprising the target RNA sequence.

In some embodiments, at least 2 (e.g., 3, 4, 5, 6, 7, 8, 9 or more) of the plurality of RNA base-binding motifs of the RNA-binding domain are joined by a linker, e.g., an amino acid linker.

In some embodiments, the RNA binding domain and the RNA editing domain are linked by a linker, e.g., an amino acid linker.

In some embodiments, the polypeptide further comprises a splicing factor.

In another aspect, the disclosure is directed to a composition comprising a polypeptide described herein, and an anti-sense oligonucleotide comprising a sequence that is complementary to the target RNA sequence.

In another aspect, the disclosure is directed to a nucleic acid encoding a polypeptide described herein.

In some embodiments, the nucleic acid is an RNA, e.g., an mRNA.

In another aspect, the disclosure is directed to a composition comprising a nucleic acid described herein, and an anti-sense oligonucleotide comprising a sequence that is complementary to the target RNA sequence.

In another aspect, the disclosure is directed to a composition comprising a nucleic acid described herein, and a nucleic acid encoding an anti-sense oligonucleotide comprising a sequence that is complementary to the target RNA sequence.

In another aspect, the disclosure is directed to an expression vector (e.g., a plasmid vector, a viral vector) comprising a nucleic acid described herein.

In another aspect, the disclosure is directed to a host cell (e.g., a bacterial host cell, a mammalian host cell) comprising a polypeptide, nucleic acid, composition, or vector described herein.

In another aspect, the disclosure is directed to a GMP-grade pharmaceutical composition comprising a polypeptide, nucleic acid, vector, composition, or host cell described herein, and a pharmaceutically acceptable excipient.

In some embodiments, a polypeptide, nucleic acid, vector, composition, pharmaceutical composition, or host cell described herein is encapsulated or formulated in a pharmaceutical carrier (e.g., a vesicle, liposome, LNP).

In another aspect, the disclosure is directed to a method of modifying (e.g., changing the sequence of) a target RNA, comprising contacting a cell, tissue or subject with a polypeptide, nucleic acid, vector, composition, host cell, or GMP-grade pharmaceutical composition described herein, in an amount and for a time sufficient for the RNA binding domain of the polypeptide to bind the target RNA in the cell, tissue or subject, and for the RNA editing domain of the polypeptide to edit the target RNA.

In some embodiments, the target RNA is a pre-mRNA or an mRNA that has secondary and/or tertiary structure.

In some embodiments, the target RNA is a pre-mRNA, e.g., an intron-exon junction of a pre-mRNA.

In some embodiments, the polypeptide alters the nucleotide sequence of the target RNA.

In some embodiments, altering comprises modifying at least 1, 2, 3, 4, 5, 6, 7, 8, 9, or 10 (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7- 10, 7-9, 7-8, 8-10, 8-9, or 9-10) nucleotides of the target RNA sequence or an RNA comprising the target sequence.

In some embodiments, altering comprises modifying a single nucleotide of the target RNA sequence or an RNA comprising the target sequence.

In some embodiments, altering comprises changing a base to another base, e.g., changes a cytosine to a uracil; an adenosine to an inosine; or a guanosine to an adenosine.

In some embodiments, altering comprises modifying an amino-acid encoding sequence of the target RNA sequence.

In some embodiments, the modification to the amino-acid encoding sequence of the target RNA sequence alters the amino acid sequence of a product polypeptide encoded by the target RNA sequence.

In some embodiments, the target RNA comprises a pre-mRNA or mRNA in a cell, tissue or subject, and the polypeptide alters (e.g., increases or decreases) secondary or tertiary structure of the pre-mRNA or mRNA.

In some embodiments, the target RNA comprises a pre-mRNA or mRNA in a cell, tissue or subject, and the polypeptide alters splicing of the pre-mRNA or mRNA.

In some embodiments, the polypeptide inhibits, e.g., eliminates, splicing of the pre-mRNA or mRNA at a splice site (e.g., a target splice site), and optionally does not inhibit splicing of the pre-mRNA or mRNA at one or more other splice site(s) (e.g., one or more non-target splice site(s)).

In some embodiments, the target RNA comprises Epstein-Barr Virus (EBV) mRNA, e.g., EBV nuclear antigen 1 (EBNA1) mRNA.

In some embodiments, the target RNA comprises Spinal Muscle Neuron 2 (SMN2) mRNA.

In some embodiments, the target RNA comprises GluA2 mRNA.

In some embodiments, the polypeptide comprises an amino acid sequence chosen from SEQ ID NOs: 13-21 or an amino acid sequence with at least 80, 85, 90, 91, 92, 93, 94, 95, 96, 97, 98, or 99% identity thereto or having no more than 1, 2, 3, 4, 5, 6, 7, 8, 9, or 10 base alterations (e.g., substitutions, deletions, or insertions) relative thereto.

In some embodiments, the RNA-binding domain binds to a target RNA sequence comprising an RNA sequence chosen from SEQ ID NOs: 22-25 or having no more than 1, 2, 3, 4, 5, 6, 7, 8, 9, or 10 base alterations relative thereto.

In another aspect, the disclosure is directed to a method of treating a disease or disorder in a subject, e.g., a human subject, comprising administering to the subject an effective amount of a polypeptide, pharmaceutical composition, nucleic acid, vector, composition, or host cell described herein, thereby treating the disease or disorder, wherein the disease or disorder is chosen from Meier-Gorlin syndrome, Seckel syndrome 4, Joubert syndrome 5, Leber congenital amaurosis 10; Charcot-Marie-Tooth disease, type 2; Charcot-Marie-Tooth disease, type 2; Usher syndrome, type 2C; Spinocerebellar ataxia 28; Spinocerebellar ataxia 28; Spinocerebellar ataxia 28; Long QT syndrome 2; Sjogren-Larsson syndrome; Hereditary fructosuria; Hereditary fructosuria; Neuroblastoma; Neuroblastoma; Kallmann syndrome 1; Kallmann syndrome 1; Kallmann syndrome 1; Metachromatic leukodystrophy, Rett syndrome, Amyotrophic lateral sclerosis type 10, Li-Fraumeni syndrome, Cystic fibrosis, Hurler Syndrome, alpha-1-antitrypsin (AlAT) deficiency, Parkinson's disease, Alzheimer's disease, albinism, Amyotrophic lateral sclerosis, Asthma, b-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (I BD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, Sturge-Weber Syndrome, and cancer.

In another aspect, the disclosure is directed to a method of treating a subject (e.g., a human subject) infected by or suspected of being infected by Epstein-Barr Virus (EBV), comprising administering to the subject an effective amount of a polypeptide, pharmaceutical composition, nucleic acid, vector, composition, or host cell described herein, thereby treating the subject infected by or suspected of being infected by Epstein-Barr Virus (EBV).

In some embodiments, the subject has mononucleosis or cancer (e.g., Burkitt lymphoma, Hodgkin's, and nasopharyngeal carcinomas).

In another aspect, the disclosure is directed to a method of treating a subject (e.g., a human subject) having Spinal Muscle Atrophy (SMA), comprising administering to the subject an effective amount of a polypeptide, pharmaceutical composition, nucleic acid, vector, composition, or host cell described herein, thereby treating the subject having SMA.

In another aspect, the disclosure is directed to a method of treating a subject (e.g., a human subject) having Amyotrophic Lateral Sclerosis (ALS), comprising administering to the subject an effective amount of a polypeptide, pharmaceutical composition, nucleic acid, vector, composition, or host cell described herein, thereby treating the subject having ALS.

### BRIEF DESCRIPTION OF THE DRAWINGS

FIGS. 1A and 1B show an illustration of an exemplary RNA editor composition: GluA2.RBD-hADARDD (1A) and an illustration of the expected resulting edit of the GluA2 mRNA sequence (1B).

FIG. 2 shows an illustration of human SMN2 splicing in a Spinal Muscle Atrophy patient.

FIG. 3 shows an exemplary RNA editor composition: SMN2.RBD-hADARDD and an illustration of the expected resulting, corrective edit of the SMN2 mRNA sequence.

FIG. 4 shows an illustration showing editing of the sequence of EBNA1 to augment the secondary structure of the viral mRNA to induce an immune response in a host, with the secondary structures as predicted by MFOLD.

### DETAILED DESCRIPTION

The invention describes RNA-editing compositions and related methods. Compositions described herein (e.g., pharmaceutical compositions) include a polypeptide comprising an RNA binding domain comprising a plurality of (e.g., 2-50, 2-30, 15-30, 16-21, 5-20, 5-15, 5-10) RNA base-binding motifs, each of which binds to an RNA base, and which are ordered in the RNA binding domain to bind to the consecutive order of the RNA bases in the target RNA sequence, linked to a heterologous RNA editing domain, e.g., a deaminase, e.g., an adenosine deaminase or a cytidine deaminase. The compositions and methods described herein may be used to modify an RNA sequence, e.g., to alter one or more of: secondary and/or tertiary structure of the RNA; splicing; the amino acid sequence of an encoded polypeptide; or the level of expression of an encoded polypeptide, or add or eliminate a stop codon (e.g., a premature stop codon). In embodiments, the RNA-binding domain binds an RNA and the RNA editing domain edits the RNA to reduce or increase the secondary and/or tertiary structure of the RNA, and/or alter splicing of the RNA. In some embodiments, the composition reduces the amount of double stranded RNA structure, e.g., to decrease an immune response to the RNA. In some embodiments, the composition increases the amount of double stranded RNA structure, e.g., to increase an immune response to the RNA. In some embodiments, the composition corrects a disease-associated mutation that causes a pathological splice product.

### Definitions

As used herein, term “domain” refers to a structure of a biomolecule that contributes to a specified function of the biomolecule. A domain may comprise a contiguous region (e.g., a contiguous sequence) or distinct, non-contiguous regions (e.g., non-contiguous sequences) of a biomolecule. Examples of protein domains include, but are not limited to, an RNA binding domain, an effector domain, an RNA editing domain.

As used herein, the term “exogenous”, when used with reference to a biomolecule (such as a nucleic acid sequence or polypeptide) means that the biomolecule was introduced into a host genome, cell or organism by human intervention. For example, a nucleic acid that is added into an existing genome, cell, tissue or subject using recombinant DNA techniques or other methods is exogenous to the existing nucleic acid sequence, cell, tissue or subject.

As used herein, the term “heterologous”, when used to describe a first element in reference to a second element means that the first element and second element do not exist in nature disposed as described. For example, a heterologous polypeptide, nucleic acid molecule, construct or sequence refers to (a) a polypeptide, nucleic acid molecule or portion of a polypeptide or nucleic acid molecule sequence that is not native to a cell in which it is expressed, (b) a polypeptide or nucleic acid molecule or portion of a polypeptide or nucleic acid molecule that has been altered or mutated relative to its native state, or (c) a polypeptide or nucleic acid molecule with an altered expression as compared to the native expression levels under similar conditions. For example, a heterologous regulatory sequence (e.g., promoter, enhancer) may be used to regulate expression of a gene or a nucleic acid molecule in a way that is different than the gene or a nucleic acid molecule is normally expressed in nature. In another example, a heterologous domain of a polypeptide or nucleic acid sequence (e.g., an RNA-binding domain of a polypeptide or nucleic acid encoding an RNA-binding domain of a polypeptide) may be disposed relative to other domains or may be a different sequence or from a different source, relative to other domains or portions of a polypeptide or its encoding nucleic acid. In certain embodiments, a heterologous nucleic acid molecule may exist in a native host cell genome but may have an altered expression level or have a different sequence or both. In other embodiments, heterologous nucleic acid molecules may not be endogenous to a host cell or host genome but instead may have been introduced into a host cell by transformation (e.g., transfection, electroporation), wherein the added molecule may integrate into the host genome or can exist as extra-chromosomal genetic material either transiently (e.g., mRNA) or semi-stably for more than one generation (e.g., episomal viral vector, plasmid or other self-replicating vector).

As used herein, the term “mutated”, “mutation” and cognates, when applied to nucleic acid sequences, means that nucleotides in a nucleic acid sequence may be inserted, deleted or changed (e.g., a point mutation) compared to a reference nucleic acid sequence (e.g., a native, wild type or non-pathological nucleic acid sequence).

As used herein, a “nucleic acid” refers to both RNA and DNA molecules including, without limitation, cDNA, genomic DNA, mRNA, tRNA, and also includes synthetic nucleic acid molecules, such as those that are chemically synthesized or recombinantly produced, such as nucleotide sequences described herein. A nucleic acid molecule can be double-stranded or single-stranded, combinations thereof, circular or linear. If single-stranded, the nucleic acid molecule can be the sense strand or the antisense strand. Nucleic acid sequences may be modified chemically or biochemically or may contain non-natural or derivatized nucleotide bases, as will be readily appreciated by those of skill in the art. Such modifications include, for example, labels, methylation, substitution of one or more naturally occurring nucleotides with an analog, inter-nucleotide modifications such as uncharged linkages (for example, methyl phosphonates, phosphotriesters, phosphoramidates, carbamates, etc.), charged linkages (for example, phosphorothioates, phosphorodithioates, etc.), pendant moieties, (for example, polypeptides), intercalators (for example, acridine, psoralen, etc.), chelators, alkylators, and modified linkages (for example, alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of a molecule. Other modifications can include, for example, analogs in which the ribose ring contains a bridging moiety or other structure such as modifications found in “locked” nucleic acids.

As used herein an “RNA binding domain” of a polypeptide is a domain of a polypeptide that specifically binds a target RNA sequence. The RNA-binding domain may comprise a plurality of RNA base-binding motifs, each of which is capable of specifically binding to an RNA base, and which are ordered in the RNA binding domain to bind to the consecutive order of the RNA bases in the target RNA sequence. As used herein, a “PUM RNA-binding motif” is a motif homologous to or derived from a RNA base-binding repeat of a Pumilio homology domain (PUM-HD). In embodiments, a PUM RNA-binding motif is at least 80% (e.g., 85%, 87%, 90%, 92%, 95%, 97%, 98%, 99% or 100%) identical to a RNA base-binding repeat of a PUM-HD and has binding specificity for a particular RNA base. In some embodiments, the PUM RNA-binding motif has a modular unit. In some embodiments, the modular unit binds to the RNA base adenine, wherein modular unit amino acid 1 is Cysteine, modular unit amino acid 2 is Tyrosine, and modular unit amino acid 5 is Glutamine. In some embodiments, the modular unit binds to the RNA base Uracil, wherein modular unit amino acid 1 is Asparagine, modular unit amino acid 2 is Tyrosine, and modular unit amino acid 5 is Glutamine. In some embodiments, the modular unit binds the RNA base Guanine, wherein modular unit amino acid 1 is Serine, modular unit amino acid 2 is Tyrosine, and modular unit amino acid 5 is Glutamic Acid. In some embodiments, the modular unit binds the RNA base Cytosine, wherein modular unit amino acid 1 is Serine, modular unit amino acid 2 is Tyrosine, and modular unit amino acid 5 is Arginine. In some embodiments, the modular unit binds Cytosine, wherein modular unit amino acid 1 is Serine, modular unit amino acid 2 is Tyrosine, and modular unit amino acid 5 is Arginine. Methods of designing and making such modular units, and RNA-binding motifs and domains are found, e.g., in Adamala et al. 2016. PNAS 113(19): E2579-E2588 and in US 2016/0238593.

As used herein, an “RNA effector” is a moiety that acts on RNA to modulate its structure and/or function, e.g., to edit the nucleotide sequence of a target RNA. An example of an RNA effector is a catalytic domain of an enzyme that edits one or more bases of a target RNA sequence (an “RNA editing” domain), e.g., a catalytic domain of a deaminase, e.g., a cytidine deaminase that edits a cytosine to a uracil, an adenosine deaminase that edits an adenosine to an inosine, or a catalytic domain of an APOBEC3A, which has been reported to have the capacity to convert G to A (e.g., as in Ahmadreza et al. 2015. PloS one 10.3: e0120089). Such enzymes include Adenosine Deaminases Acting on RNA (ADARs) (e.g., human ADAR 1, human ADAR2, human ADAR3, or human ADAR4); Adenosine Deaminases Acting on tRNAs (ADATs), Cytosine Deaminases Acting on RNA (CDARs), APOBEC, APOBEC3A A3A, TadA or CDA.

As used herein, the term “host” cell, as used herein, refer to a cell and/or its genome into which protein and/or genetic material has been introduced. The term is intended to refer not only to the particular subject cell and/or genome, but to the progeny of such a cell and/or the genome of the progeny of such a cell. Because certain modifications may occur in succeeding generations due to either mutation or environmental influences, such progeny may not, in fact, be identical to the parent cell, but are still included within the scope of the term “host cell” as used herein. A host genome or host cell may be an isolated cell or cell line grown in culture, or genomic material isolated from such a cell or cell line, or may be a host cell or host genome which composing living tissue or an organism.

As used herein, the terms “effective” or “sufficient” amount and/or time of a composition described herein refer to a quantity and/or time sufficient to, when administered to a cell, tissue or subject, including a mammal (e.g., a human), effect the desired results, including effects at the cellular level, tissue level, or clinical results, and, as such, an “effective” or “sufficient” or synonym thereto depends upon the context in which it is being applied. For example, in the context of modulating RNA structure it is an amount of the composition sufficient to achieve a change to RNA structure as compared to the response obtained without administration of the composition (e.g., polypeptide, nucleic acid, vector, etc.). The amount of a given composition described herein that will correspond to such an amount will vary depending upon various factors, such as the given agent, the pharmaceutical formulation, the route of administration, the type of disease or disorder, the identity of the cell, tissue or subject (e.g., age, sex, weight) or host being treated, and the like, but can nevertheless be routinely determined by one skilled in the art. Also, as used herein, a “therapeutically effective amount” of a composition of the present disclosure is an amount that results in a beneficial or desired result in a subject as compared to a control. As defined herein, a therapeutically effective amount of a composition of the present disclosure may be readily determined by one of ordinary skill by routine methods known in the art. Dosage regimen may be adjusted to provide the optimum therapeutic response.

As used herein, the terms “increasing” and “decreasing” refer to modulating resulting in, respectively, greater or lesser amounts, of function, expression, or activity of a metric relative to a reference. For example, subsequent to administration of composition described herein, an RNA function and/or structure (e.g., expression or regulatory activity) as described herein may be increased or decreased in a cell, tissue or subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount prior to administration. Generally, the metric is measured subsequent to administration at a time that the administration has had the recited effect, e.g., hours, days, at least one week, one month, 3 months, or 6 months, or after a treatment regimen has begun in the context of a subject.

As used herein, a “pharmaceutical composition” or “pharmaceutical preparation” is a composition or preparation having pharmacological activity or other direct effect in the mitigation, treatment, or prevention of disease, and/or a finished dosage form or formulation thereof and which is indicated for human use. A pharmaceutical composition is typically GMP grade, i.e., it meets US regulatory (FDA) specifications for compositions to be used in humans. For example, a GMP-grade composition is typically tested for endotoxin and meets a release criterion of having less than a specified amount of endotoxin.

“Treatment” and “treating,” as used herein, refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

### RNA-Binding Domain

An RNA-binding domain of a polypeptide described herein specifically binds a target RNA sequence. The RNA-binding domain may comprise a plurality of RNA base-binding motifs, each of which is capable of specifically binding to an RNA base, and which motifs are ordered in the RNA binding domain such as to bind to the consecutive order of the RNA bases in the target RNA sequence. An RNA-binding motif may be based on a sequence homologous to or derived from a RNA base-binding repeat of a Pumilio homology domain (PUM-HD) (a “PUM RNA-binding motif”). In embodiments, a PUM RNA-binding motif is at least 80% (e.g., 85%, 87%, 90%, 92%, 95%, 97%, 98%, 99% or 100%) identical to a RNA base-binding motif of a PUM-HD and has binding specificity for a particular RNA base. In PUM RNA-binding motifs, specificity for a target RNA base is engineered based on conserved positions on topologically equivalent protein surfaces, governed by hydrogen bonds or van der Waals interactions, that bind the Watson-Crick edge of the nucleic acids. These topologies are targeted to RNA using glutamate and serine at the 1st and 5th positions to recognize guanine; glutamine and cysteine/serine to recognize adenine; and glutamine and asparagine to recognize uracil. Methods of designing and making such modular units, and RNA-binding motifs and domains are found, e.g., in Lu et al. 2009. Curr Opin Struct Biol. 19(1): 110-115; Adamala et al. 2016. PNAS 113(19): E2579-E2588; and US 2016/0238593.

In embodiments, the RNA binding domain has at least 80% identity (e.g., at least 85% identity, at least 87% identity, at least 90% identity, at least 92% identity, at least 95% identity, at least 97% identity, at least 98% identity, or 99% identity) and less than 100% identity to a corresponding amino acid sequence of a wild type PUM-HD, e.g., wild type human PUM1-HD. In one example, HsPUM1-HD RNA-binding motifs to target for mutagenesis and the correlative recognized nucleotides are shown in Table A (from Wang et al. 2002. Cell 110(4):501-12).

TABLE A

| | AA sequence | | |
|---|---|---|---|
| Repeat | of repeat | Target Residue | Nucleotide |
| R1 | HIMEFSQDQHGS | Ser863, Arg864, | A |
| | RFIQLKLERATP | Gln867 | |
| | AERQLVFNEILQ | | |
| R3 | HVLSLALQMYGC | Cys935, Arg936, | A |
| | RVIQKALEFIPS | Gln939 | |
| | DQQNEMVRELDG | | |
| R5 | QVFALSTHPYGC | Cys1007, Arg1008, | A |
| | RVIQRILEHCLP | Gln1011 | |
| | DQTLPILEELHQ | | |
| R7 | NVLVLSQHKFAS | Ser1079, Ans1080, | G |
| | NVVEKCVTHASR | Glu1083 | |
| | TERAVLIDEVCT | | |
| | MNDGPHS | | |
| R2 | AAYQLMVDVFGN | Asn899, Tyr900, | U |
| | YVIQKFFEFGSL | Gln903 | |
| | EQKLALAERIRG | | |
| R4 | HVLKCVKDQNGN | Asn971, His972, | U |
| | HVVQKCIECVQP | Gln975 | |
| | QSLQFIIDAFKG | | |
| R6 | HTEQLVQDQYGN | Asn1043, Tyr1044, | U |
| | YVIQHVLEHGRP | Gln1047 | |
| | EDKSKIVAEIRG | | |
| R8 | ALYTMMKDQYAN | Tyr1123, Asn 1122, | U |
| | YVVQKMIDVAEP | Gln1126 | |
| | GQRKIVMHKIRP | | |
| | HIATLRKYTYGK | | |
| | HILAKLEKYYMK | | |
| | NGVDLG | | |

For example, to bind an uracil (U) rather than guanine (G) in repeat 7, the following amino acid residue changes are made: E1083Q, S1079N and N1080Y, as described, e.g., in Cheong and Tanaka. 2006. PNAS vol. 103, 37: 13635-9.

The engineered RNA-binding domain is designed to bind a target RNA sequence. Typically, the RNA binding domain binds a target sequence of 2-50 RNA nucleotides (e.g., 2-50 nucleotides (e.g., 2-50, 2-40, 2-30, 2-25, 2-24, 2-23, 2-22, 2-21, 2-20, 2-19, 2-18, 2-17, 2-16, 2-15, 2-14, 2-13, 2-12, 2-11, 2-10, 2-9, 2-8, 5-50, 5-40, 5-30, 5-25, 5-24, 5-23, 5-22, 5-21, 5-20, 5-19, 5-18, 5-17, 5-16, 5-15, 5-14, 5-13, 5-12, 5-11, 5-10, 5-9, 5-8, 10-50, 10-40, 10-30, 10-25, 10-24, 10-23, 10-22, 10-21, 10-20, 10-19, 10-18, 10-17, 10-16, 10-15, 10-14, 10-13, 10-12, 10-11, 15-50, 15-40, 15-30, 15-25, 15-24, 15-23, 15-22, 15-21, 15-20, 15-19, 15-18, 15-17, 15-16, 16-50, 16-40, 16-30, 16-25, 16-24, 16-23, 16-22, 16-21, 16-20, 16-19, 16-18, 16-17, 17-50, 17-40, 17-30, 17-25, 17-24, 17-23, 17-22, 17-21, 17-20, 17-19, 17-18, 18-50, 18-40, 18-30, 18-25, 18-24, 18-23, 18-22, 18-21, 18-20, 18-19, 19-50, 19-40, 19-30, 19-25, 19-24, 19-23, 19-22, 19-21, 19-20, 20-50, 20-40, 20-30, 20-25, 20-24, 20-23, 20-22, 20-21, 21-50, 21-40, 21-30, 21-25, 21-24, 21-23, 21-22, 25-50, 25-40, 25-30, 30-50, 30-40, or 40-50, 3-20, 3-15, 3-10, 3-9, 3-8, 4-12, 4-10, 4-9, 4-8, 5-10, 5-9, 5-8 nucleotides). In some embodiments, the RNA binding domain binds a target sequence of 16-21 RNA nucleotides. In some embodiments, the RNA binding domain binds at least 16 RNA nucleotides (and optionally no more than 30, 29, 28, 27, 26, 25, 24, 23, 22, or 21 RNA nucleotides). The plurality of RNA base-binding motifs may include at least 3 (e.g., at least 4 at least 5, at least 6, at least 7, at least 8, at least 9, between 2-20, between 2-15, between 2-10, between 2-8, between 3-20, between 3-15, between 3-10, between 3-8, between 4-8, up to 25, up to 30) PUM RNA-binding motifs. In some embodiments, the RNA binding domain comprises 2-50, 2-40, 2-30, 2-25, 2-24, 2-23, 2-22, 2-21, 2-20, 2-19, 2-18, 2-17, 2-16, 2-15, 2-14, 2-13, 2-12, 2-11, 2-10, 2-9, 2-8, 5-50, 5-40, 5-30, 5-25, 5-24, 5-23, 5-22, 5-21, 5-20, 5-19, 5-18, 5-17, 5-16, 5-15, 5-14, 5-13, 5-12, 5-11, 5-10, 5-9, 5-8, 10-50, 10-40, 10-30, 10-25, 10-24, 10-23, 10-22, 10-21, 10-20, 10-19, 10-18, 10-17, 10-16, 10-15, 10-14, 10-13, 10-12, 10-11, 15-50, 15-40, 15-30, 15-25, 15-24, 15-23, 15-22, 15-21, 15-20, 15-19, 15-18, 15-17, 15-16, 16-50, 16-40, 16-30, 16-25, 16-24, 16-23, 16-22, 16-21, 16-20, 16-19, 16-18, 16-17, 17-50, 17-40, 17-30, 17-25, 17-24, 17-23, 17-22, 17-21, 17-20, 17-19, 17-18, 18-50, 18-40, 18-30, 18-25, 18-24, 18-23, 18-22, 18-21, 18-20, 18-19, 19-50, 19-40, 19-30, 19-25, 19-24, 19-23, 19-22, 19-21, 19-20, 20-50, 20-40, 20-30, 20-25, 20-24, 20-23, 20-22, 20-21, 21-50, 21-40, 21-30, 21-25, 21-24, 21-23, 21-22, 25-50, 25-40, 25-30, 30-50, 30-40, or 40-50 PUM RNA-binding motifs, e.g., a number of PUM RNA-binding motifs corresponding to the number of RNA nucleotides bound (e.g., the length of the target RNA sequence).

In some embodiments, an RNA-binding domain binds a target RNA sequence in an mRNA encoded by the GluA2 (e.g., human GluA2) gene. In some embodiments, the RNA-binding domain binds to a target RNA sequence comprising nucleotides corresponding to 1537-1552 of the human GluA2 gene, or a nucleic acid sequence within 50 bases of nucleotides 1537-1552 in Reference sequence NM_000826.

In some embodiments, an RNA-binding domain binds a target RNA sequence in an mRNA encoded by the SMN2 (e.g., human SMN2) gene. In some embodiments, the RNA-binding domain binds to a target RNA sequence comprising nucleotides corresponding to 31,995-32,010 of the human SMN2 gene, or a nucleic acid sequence within 50 bases of nucleotides 31,995-32,010 in Reference sequence NM-022876.

An RNA binding domain described herein may be between 90-500 amino acid residues, e.g., between 90-450 amino acid residues, between 90-400 amino acid residues, between 90-350 amino acid residues, between 90-300 amino acid residues, between 120-400 amino acid residues. An RNA binding domain may bind an RNA sequence, e.g., an mRNA sequence, e.g., an mRNA sequence that folds into a secondary or tertiary structure, e.g., a double stranded RNA sequence. An RNA binding domain may bind an RNA sequence, e.g., an mRNA sequence, e.g., an mRNA sequence comprising a disease-associated mutation, e.g., a point mutation.

In some embodiments, a PUM RNA-binding motif describes herein binds to cytosine. More particularly, PUM RNA-binding motifs may be engineered to bind cytosine, e.g., by the methods of U.S. Ser. No. 10/233,218B2, which is hereby incorporated by reference. In some embodiments, an RNA-binding domain comprises one or more PUM RNA-binding motifs that binds to cytosine. For example, an PUM RNA binding motif that binds cytosine may comprise a sequence with the formula X₁X₂X₃X₄X₅X₆X₇X₈X₉X₁₀X₁₁ wherein:

X₁ is glutamine (Q), X₂ is histidine (H); X₃ is glycine (G); X₄ is selected from the group including glycine (G), alanine (A), serine (S), threonine (T) and cysteine (C); X₅ is arginine (R); X₆ is phenylalanine (F); X₇ is isoleucine (I); X₈ is arginine (R); X₉ is leucine (L); X₁₀ is lysine (K); and X₁₁ is leucine (L); or

X₁ is valine (V); X₂ is phenylalanine (F); X₃ is glycine (G); X₄ is selected from the group including glycine (G), alanine (A), serine (S), threonine (T) and cysteine (C); X₅ is tyrosine (Y); X₆ is valine (V); X₇ is isoleucine (I); X₈ is arginine (R); X₉ is lysine (K); X₁₀ is phenylalanine (F); and X₁₁ is phenylalanine (F); or

X₁ is methionine (M); X₂ is tyrosine (Y); X₃ is glycine (G); X₄ is selected from the group including glycine (G), alanine (A), serine (S), threonine (T) and cysteine (C); X₅ is arginine (R); X₆ is valine (V); X₇ is isoleucine (I); X₈ is arginine (R); X₉ is lysine (K); X₁₀ is alanine (A); and X₁₁ is leucine (L); or

X₁ is glutamine (Q); X₂ is asparagine (N); X₃ is glycine (G); X₄ is selected from the group including glycine (G), alanine (A), serine (S), threonine (T) and cysteine (C); X₅ is histidine (H); X₆ is valine (V); X₇ is valine (V); X₈ is arginine (R); X₉ is lysine (K); X₁₀ is cysteine (C); and X₁₁ is isoleucine (I); or

X₁ is proline (P); X₂ is tyrosine (Y); X₃ is glycine (G); X₄ is selected from the group including glycine (G), alanine (A), serine (S), threonine (T) and cysteine (C); X₅ is arginine (R); X₆ is valine (V); X₇ is isoleucine (I); X₈ is arginine; (R); X₉ is arginine (R); X₁₀ is isoleucine (I); and X₁₁ is leucine (L); or

X₁ is glutamine (Q); X₂ is tyrosine (Y); X₃ is glycine (G); X₄ is selected from the group including glycine (G), alanine (A), serine (S), threonine (T) and cysteine (C); X₅ is tyrosine (Y); X₆ is valine (V); X₇ is isoleucine (I); X₈ is arginine; (R); X₉ is histidine (H); X₁₀ is valine (V); and X₁₁ is leucine (L); or

X₁ is lysine (K); X₂ is phenylalanine (F); X₃ is alanine (A); X₄ is selected from the group including glycine (G), alanine (A), serine (S), threonine (T) and cysteine (C); X₅ is asparagine (N); X₆ is valine (V); X₇ is valine (V); X₈ is arginine; (R); X₉ is lysine (K); X₁₀ is cysteine (C); and X₁₁ is valine (V); or

X₁ is glutamine (Q); X₂ is tyrosine (Y); X₃ is alanine (A); X₄ is selected from the group including glycine (G), alanine (A), serine (S), threonine (T) and cysteine (C); X₅ is tyrosine (Y); X₆ is valine (V); X₇ is valine (V); X₈ is arginine; (R); X₉ is lysine (K); X₁₀ is methionine (M); and X₁₁ is isoleucine (I).

For example, an RNA binding motif that binds cytosine may comprise the amino acid sequence QYGGYVIRHVL (SEQ ID NO: 100). In some embodiments, an RNA binding domain comprising an RNA-binding motif that binds cytosine may comprise the amino acid sequence:

(SEQ ID NO: 101)
GRSRLLEDFRNNRYPNLQLREIAGHIMEFSQDQHGSRFIQLKLERATPAE
RQLVFNEILQAAYQLMVDVFGNYVIQKFFEFGSLEQKLALAERIRGHVLS
LALQMYGCRVIQKALEFIPSDQQVINEMVRELDGHVLKCVKDQNGNHVVQ
KCIECVQPQSLQFIIDAFKGQVFALSTHPYGCRVIQRILEHCLPDQTLPI
LEELHQHTEQLVQDQYGGYVIRHVLEHGRPEDKSKIVAEIRGNVLVLSQH
KFASNVVEKCVTHASRTERAVLIDEVCTMNDGPHSALYTMMKDQYANYVV
QKMIDVAEPGQRKIVMHKIRPHIATLRKYTYGKHILAKLEKYYMKNGVDL
G

Exemplary RNA-Binding Domains

Exemplary RNA-binding domains, e.g., comprising a plurality of RNA binding motifs (e.g., a plurality of PUM RNA-binding motifs or sequences homologous to or derived from a PUM-HD), include the RNA-binding domains of SEQ ID NOs: 13 or 15-21, or as encoded by SEQ ID NOs: 4 or 6-12. In some embodiments, an RNA-binding domain comprises an amino acid sequence with at least 80, 85, 90, 91, 92, 93, 94, 95, 96, 97, 98, or 99% identity to the RNA-binding domain of SEQ ID NOs: 13 or 15-21 (or comprising no more than 1, 2, 3, 4, 5, 6, 7, 8, 9, or 10 base alterations relative thereto), or are encoded by a nucleic acid sequence with at least 80, 85, 90, 91, 92, 93, 94, 95, 96, 97, 98, or 99% identity to the RNA-binding domain encoding sequence of SEQ ID NOs: 4 or 6-12. In some embodiments, an RNA-binding domain comprises one or more RNA-binding motifs from a first exemplary RNA-binding domain and one or more RNA-binding motifs from a second exemplary RNA-binding domain.

Dual PUF Design with (G4S)3 Linker (Wildtype PUF Targeting Sequence)

mRNA sequence:
(SEQ ID NO: 4)
augggcaggagcaggcuuuuggaagauuuucgaaacaaccgCuaccccaauuuacaacugcgggagauugcugga
cauauaauggaauuuucccaagaccagcauggguccagauucauucagcugaaacuggagcgugccacaccagcug
agcgccagcuugucuucaaugaaauccuccaggcugccuaccaacucaugguggauguguuugguaauuacgucau
ucagaaguucuuugaauuuggcagucuugaacagaagcuggcuuuggcagaacggauucgaggccacguccuguc
auuggcacuacagauguauggcugccguguuauccagaaagcucuugaguuuauuccuucagaccagcagaaugag
augguucgggaacuagauggccaugucuugaagugugugaaagaucagaauggcaaucacgugguucagaaaugc
auugaauguguacagccccagucuuugcaauuuaucaucgaugcguuuaagggacagguauuugccuuauccacac
auccuuauggcugccgagugauucagagaauccuggagcacugucucccugaccagacacucccuauuuuagagga
gcuucaccagcacacagagcagcuuguacaggaucaauauggaaauuauguaauccaacauguacuggagcacggu
cguccugaggauaaaagcaaaauuguagcagaaauccgaggcaauguacuuguauugagucagcacaaauuugcaa
gcaauguuguggagaaguguguuacucacgccucacguacggagcgcgcugugcucaucgaugaggugugcacca
ugaacgacgguccccacagugccuuauacaccaugaugaaggaccaguaugccaacuacgugguccagaagaugau
ugacguggcggagccaggccagcggaagaucgucaugcauaagauccggccccacaucgcaacucuucguaaguac
accuauggcaagcacauucuggccaagcuggagaaguacuacaugaagaacgguguugacuuagggGGAGGU
GGCGGAUCGGGAGGUGGCGGAUCGGGAGGUGGCGGAUCGggcaggagcaggcuuuu
ggaagauuuucgaaacaaccgCuaccccaauuuacaacugcgggagauugcuggacauauaauggaauuuucccaa
gaccagcauggguccagauucauucagcugaaacuggagcgugccacaccagcugagcgccagcuugucuucaaug
aaauccuccaggcugccuaccaacucaugguggauguguuugguaauuacgucauucagaaguucuuugaauuug
gcagucuugaacagaagcuggcuuuggcagaacggauucgaggccacguccugucauuggcacuacagauguaug
gcugccguguuauccagaaagcucuugaguuuauuccuucagaccagcagaaugagaugguucgggaacuagaug
gccaugucuugaagugugugaaagaucagaauggcaaucacgugguucagaaaugcauugaauguguacagcccca
gucuuugcaauuuaucaucgaugcguuuaagggacagguauuugccuuauccacacauccuuauggcugccgagu
gauucagagaauccuggagcacugucucccugaccagacacucccuauuuuagaggagcuucaccagcacacagagc
agcuuguacaggaucaauauggaaauuauguaauccaacauguacuggagcacggucguccugaggauaaaagcaa
aauuguagcagaaauccgaggcaauguacuuguauugagucagcacaaauuugcaagcaauguuguggagaagug
uguuacucacgccucacguacggagcgcgcugugcucaucgaugaggugugcaccaugaacgacgguccccacagu
gccuuauacaccaugaugaaggaccaguaugccaacuacgugguccagaagaugauugacguggcggagccaggcc
agcggaagaucgucaugcauaagauccggccccacaucgcaacucuucguaaguacaccuauggcaagcacauucug
gccaagcuggagaaguacuacaugaagaacgguguugacuuaggguga
Protein sequence:
(SEQ ID NO: 13)
MGRSRLLEDFRNNRYPNLQLREIAGHIMEFSQDQHGSRFIQLKLERATPAERQLV
FNEILQAAYQLMVDVFGNYVIQKFFEFGSLEQKLALAERIRGHVLSLALQMYGC
RVIQKALEFIPSDQQNEMVRELDGHVLKCVKDQNGNHVVQKCIECVQPQSLQFII
DAFKGQVFALSTHPYGCRVIQRILEHCLPDQTLPILEELHQHTEQLVQDQYGNYV
IQHVLEHGRPEDKSKIVAEIRGNVLVLSQHKFASNVVEKCVTHASRTERAVLIDE
VCTMNDGPHSALYTMMKDQYANYVVQKMIDVAEPGQRKIVMHKIRPHIATLR
KYTYGKHILAKLEKYYMKNGVDLGGGGGSGGGGSGGGGSGRSRLLEDFRNNR
YPNLQLREIAGHIMEFSQDQHGSRFIQLKLERATPAERQLVFNEILQAAYQLMVD
VFGNYVIQKFFEFGSLEQKLALAERIRGHVLSLALQMYGCRVIQKALEFIPSDQQ
NEMVRELDGHVLKCVKDQNGNHVVQKCIECVQPQSLQFIIDAFKGQVFALSTHP
YGCRVIQRILEHCLPDQTLPILEELHQHTEQLVQDQYGNYVIQHVLEHGRPEDKS
KIVAEIRGNVLVLSQHKFASNVVEKCVTHASRTERAVLIDEVCTMNDGPHSALY
TMMKDQYANYVVQKMIDVAEPGQRKIVMHKIRPHIATLRKYTYGKHILAKLEK
YYMKNGVDLG*

Domain of ADAR2DD (Amino Acids 299-701) with E488Q Mutations

mRNA sequence:
(SEQ ID NO: 5)
AUGCUCCACCUCGACCAAACACCCAGCAGACAGCCUAUCCCUUCCGAAGGA
CUGcagcugcauuuaccgcagguuuuagcugacgcugucucacgccugguccuggguaaguuuggugaucugac
cgacaacuucuccuccccucacgcucgcagaaaagugcuggcuggagucgucaugacaacaggcacagauguuaaa
gaugccaaggugauaaguguuucuacaggaggcaaauguauuaauggugaauacaugagugaucguggccuugca
uuaaaugacugccaugcagaaauaauaucucggagauccuugcucagauuucuuuauacacaacuugagcuuuacu
uaaauaacaaagaugaucaaaaaagauccaucuuucagaaaucagagcgagggggguuuaggcugaaggagaaugu
ccaguuucaucuguacaucagcaccucucccuguggagaugccagaaucuucucaccacaugagccaauccuggaa
gaaccagcagauagacacccaaaucguaaagcaagaggacagcuacggaccaaaauagagucuggucaggggacgau
uccagugcgcuccaaugcgagcauccaaacgugggacggggugcugcaaggggagcggcugcucaccauguccugc
agugacaagauugcacgcuggaacguggugggcauccagggaucacugcucagcauuuucguggagcccauuuac
uucucgagcaucauccugggcagccuuuaccacggggaccaccuuuccagggccauguaccagcggaucuccaaca
uagaggaccugccaccucucuacacccucaacaagccuuugcucaguggcaucagcaaugcagaagcacggcagcca
gggaaggcccccaacuucagugucaacuggacgguaggcgacuccgcuauugaggucaucaacgccacgacuggga
aggaugagcugggccgcgcgucccgccuguguaagcacgcguuguacugucgcuggaugcgugugcacggcaagg
uucccucccacuuacuacgcuccaagauuaccaagcccaacguguaccaugaguccaagcuggcggcaaaggaguac
caggccgccaaggcgcgucuguucacagccuucaucaaggcggggcugggggccuggguggagaagcccaccgagc
aggaccaguucucacucacgCCUUGA
Protein sequence:
(SEQ ID NO: 14)
MLHLDQTPSRQPIPSEGLQLHLPQVLADAVSRLVLGKFGDLTDNFSSPHARRKVL
AGVVMTTGTDVKDAKVISVSTGGKCINGEYMSDRGLALNDCHAEIISRRSLLRFL
YTQLELYLNNKDDQKRSIFQKSERGGFRLKENVQFHLYISTSPCGDARIFSPHEPI
LEEPADRHPNRKARGQLRTKIESGQGTIPVRSNASIQTWDGVLQGERLLTMSCSD
KIARWNVVGIQGSLLSIFVEPIYFSSIILGSLYHGDHLSRAMYQRISNIEDLPPLYTL
NKPLLSGISNAEARQPGKAPNFSVNWTVGDSAIEVINATTGKDELGRASRLCKHA
LYCRWMRVHGKVPSHLLRSKITKPNVYHESKLAAKEYQAAKARLFTAFIKAGL
GAWVEKPTEQDQFSLTP*

### Fusion Polypeptide of Dual PUF Design Fused to ADAR2DD (Wildtype PUF Targeting Sequence)

mRNA sequence:
(SEQ ID NO: 6)
AUGGACUAUAAGGACCACGACGGAGACUACAAGGAUCAUGAUAUUGAUUA
CAAAGACGAUGACGAUAAGAUGGCCCCAAAGAAGAAGCGGAAGGUCGGUA
UCCACGGAGUCCCAGCAGCCCUCCACCUCGACCAAACACCCAGCAGACAGC
CUAUCCCUUCCGAAGGACUGcagcugcauuuaccgcagguuuuagcugacgcugucucacgccugg
uccuggguaaguuuggugaucugaccgacaacuucuccuccccucacgcucgcagaaaagugcuggcuggagucgu
caugacaacaggcacagauguuaaagaugccaaggugauaaguguuucuacaggaggcaaauguauuaauggugaa
uacaugagugaucguggccuugcauuaaaugacugccaugcagaaauaauaucucggagauccuugcucagauuuc
uuuauacacaacuugagcuuuacuuaaauaacaaagaugaucaaaaaagauccaucuuucagaaaucagagcgaggg
ggguuuaggcugaaggagaauguccaguuucaucuguacaucagcaccucucccuguggagaugccagaaucuuc
ucaccacaugagccaauccuggaagaaccagcagauagacacccaaaucguaaagcaagaggacagcuacggaccaaa
auagagucuggucaggggacgauuccagugcgcuccaaugcgagcauccaaacgugggacggggugcugcaaggg
gagcggcugcucaccauguccugcagugacaagauugcacgcuggaacguggugggcauccagggaucacugcuca
gcauuuucguggagcccauuuacuucucgagcaucauccugggcagccuuuaccacggggaccaccuuuccagggc
cauguaccagcggaucuccaacauagaggaccugccaccucucuacacccucaacaagccuuugcucaguggcauca
gcaaugcagaagcacggcagccagggaaggcccccaacuucagugucaacuggacgguaggcgacuccgcuauuga
ggucaucaacgccacgacugggaaggaugagcugggccgcgcgucccgccuguguaagcacgcguuguacugucgc
uggaugcgugugcacggcaagguucccucccacuuacuacgcuccaagauuaccaagcccaacguguaccaugagu
ccaagcuggcggcaaaggaguaccaggccgccaaggcgcgucuguucacagccuucaucaaggcggggcugggggc
cuggguggagaagcccaccgagcaggaccaguucucacucacgCCUGGAGGUGGCGGAUCGGGAG
GUGGCGGAUCGGGAGGUGGCGGAUCGggcaggagcaggcuuuuggaagauuuucgaaacaac
cgCuaccccaauuuacaacugcgggagauugcuggacauauaauggaauuuucccaagaccagcauggguccagau
ucauucagcugaaacuggagcgugccacaccagcugagcgccagcuugucuucaaugaaauccuccaggcugccua
ccaacucaugguggauguguuugguaauuacgucauucagaaguucuuugaauuuggcagucuugaacagaagcu
ggcuuuggcagaacggauucgaggccacguccugucauuggcacuacagauguauggcugccguguuauccagaa
agcucuugaguuuauuccuucagaccagcagaaugagaugguucgggaacuagauggccaugucuugaagugugu
gaaagaucagaauggcaaucacgugguucagaaaugcauugaauguguacagccccagucuuugcaauuuaucauc
gaugcguuuaagggacagguauuugccuuauccacacauccuuauggcugccgagugauucagagaauccuggag
cacugucucccugaccagacacucccuauuuuagaggagcuucaccagcacacagagcagcuuguacaggaucaaua
uggaaauuauguaauccaacauguacuggagcacggucguccugaggauaaaagcaaaauuguagcagaaauccga
ggcaauguacuuguauugagucagcacaaauuugcaagcaauguuguggagaaguguguuacucacgccucacgu
acggagcgcgcugugcucaucgaugaggugugcaccaugaacgacgguccccacagugccuuauacaccaugauga
aggaccaguaugccaacuacgugguccagaagaugauugacguggcggagccaggccagcggaagaucgucaugca
uaagauccggccccacaucgcaacucuucguaaguacaccuauggcaagcacauucuggccaagcuggagaaguacu
acaugaagaacgguguugacuuagggGGAGGUGGCGGAUCGGGAGGUGGCGGAUCGGGA
GGUGGCGGAUCGggcaggagcaggcuuuuggaagauuuucgaaacaaccgCuaccccaauuuacaacugc
gggagauugcuggacauauaauggaauuuucccaagaccagcauggguccagauucauucagcugaaacuggagcg
ugccacaccagcugagcgccagcuugucuucaaugaaauccuccaggcugccuaccaacucaugguggauguguuu
gguaauuacgucauucagaaguucuuugaauuuggcagucuugaacagaagcuggcuuuggcagaacggauucga
ggccacguccugucauuggcacuacagauguauggcugccguguuauccagaaagcucuugaguuuauuccuuca
gaccagcagaaugagaugguucgggaacuagauggccaugucuugaagugugugaaagaucagaauggcaaucacg
ugguucagaaaugcauugaauguguacagccccagucuuugcaauuuaucaucgaugcguuuaagggacagguau
uugccuuauccacacauccuuauggcugccgagugauucagagaauccuggagcacugucucccugaccagacacu
cccuauuuuagaggagcuucaccagcacacagagcagcuuguacaggaucaauauggaaauuauguaauccaacau
guacuggagcacggucguccugaggauaaaagcaaaauuguagcagaaauccgaggcaauguacuuguauugaguc
agcacaaauuugcaagcaauguuguggagaaguguguuacucacgccucacguacggagcgcgcugugcucaucga
ugaggugugcaccaugaacgacgguccccacagugccuuauacaccaugaugaaggaccaguaugccaacuacgug
guccagaagaugauugacguggcggagccaggccagcggaagaucgucaugcauaagauccggccccacaucgcaa
cucuucguaaguacaccuauggcaagcacauucuggccaagcuggagaaguacuacaugaagaacgguguugacuu
agggAGCGGCGGCAAGCGGCCCGCCGCCACCAAGAAGGCCGGCCAGGCCAAG
AAGAAGAAGuga
Protein sequence:
(SEQ ID NO: 15)
MDYKDHDGDYKDHDIDYKDDDDKMAPKKKRKVGIHGVPAALHLDQTPSRQPI
PSEGLQLHLPQVLADAVSRLVLGKFGDLTDNFSSPHARRKVLAGVVMTTGTDV
KDAKVISVSTGGKCINGEYMSDRGLALNDCHAEIISRRSLLRFLYTQLELYLNNK
DDQKRSIFQKSERGGFRLKENVQFHLYISTSPCGDARIFSPHEPILEEPADRHPNRK
ARGQLRTKIESGQGTIPVRSNASIQTWDGVLQGERLLTMSCSDKIARWNVVGIQG
SLLSIFVEPIYFSSIILGSLYHGDHLSRAMYQRISNIEDLPPLYTLNKPLLSGISNAEA
RQPGKAPNFSVNWTVGDSAIEVINATTGKDELGRASRLCKHALYCRWMRVHGK
VPSHLLRSKITKPNVYHESKLAAKEYQAAKARLFTAFIKAGLGAWVEKPTEQDQ
FSLTPGGGGSGGGGSGGGGSGRSRLLEDFRNNRYPNLQLREIAGHIMEFSQDQH
GSRFIQLKLERATPAERQLVFNEILQAAYQLMVDVFGNYVIQKFFEFGSLEQKLA
LAERIRGHVLSLALQMYGCRVIQKALEFIPSDQQNEMVRELDGHVLKCVKDQNG
NHVVQKCIECVQPQSLQFIIDAFKGQVFALSTHPYGCRVIQRILEHCLPDQTLPILE
ELHQHTEQLVQDQYGNYVIQHVLEHGRPEDKSKIVAEIRGNVLVLSQHKFASNV
VEKCVTHASRTERAVLIDEVCTMNDGPHSALYTMMKDQYANYVVQKMIDVAE
PGQRKIVMHKIRPHIATLRKYTYGKHILAKLEKYYMKNGVDLGGGGGSGGGGS
GGGGSGRSRLLEDFRNNRYPNLQLREIAGHIMEFSQDQHGSRFIQLKLERATPAE
RQLVFNEILQAAYQLMVDVFGNYVIQKFFEFGSLEQKLALAERIRGHVLSLALQ
MYGCRVIQKALEFIPSDQQNEMVRELDGHVLKCVKDQNGNHVVQKCIECVQPQ
SLQFIIDAFKGQVFALSTHPYGCRVIQRILEHCLPDQTLPILEELHQHTEQLVQDQY
GNYVIQHVLEHGRPEDKSKIVAEIRGNVLVLSQHKFASNVVEKCVTHASRTERA
VLIDEVCTMNDGPHSALYTMMKDQYANYVVQKMIDVAEPGQRKIVMHKIRPHI
ATLRKYTYGKHILAKLEKYYMKNGVDLGSGGKRPAATKKAGQAKKKK*

Dual PUF design with (G4S)3 linker targeted towards nucleotides 1537-1552 of the human GluA2 (Reference sequence NM_000826) nucleotide sequence (aucaugaucaagaagc (SEQ ID NO: 22))

mRNA sequence:
(SEQ ID NO: 7)
augGGCCGCAGCCGCCUUUUGGAAGAUUUUCGAAACAACCGGUACCCCAAU
UUACAACUGCGGGAGAUUGCCGGACAUAUAAUGGAAUUUUCCCAAGACCA
GCAUGGGUCCAGAUUCAUUCGCCUGAAACUGGAGCGUGCCACACCAGCUG
AGCGCCAGCUUGUCUUUAAUGAAAUCCUCCAGGCUGCCUACCAACUCAUGG
UGGAUGUGUUUGGUAGUUACGUCAUUGAGAAGUUCUUUGAAUUUGGCAGU
CUUGAACAGAAGCUGGCUUUGGCAGAACGGAUUCGAGGUCACGUCCUGUC
AUUGGCACUACAGAUGUAUGGCUGCCGUGUUAUCCAGAAAGCUCUUGAGU
UUAUUCCUUCAGACCAGCAGAAUGAGAUGGUUCGGGAACUAGAUGGCCAU
GUCUUGAAGUGUGUGAAAGAUCAGAAUGGCUGUCACGUGGUUCAGAAAUG
CAUUGAAUGUGUACAGCCCCAGUCUUUGCAAUUUAUCAUCGAUGCGUUUA
AGGGCCAGGUAUUUGCCUUAUCCACACAUCCUUAUGGCUCCCGAGUGAUU
GAGAGAAUCCUGGAGCACUGUCUCCCUGACCAGACACUCCCUAUUUUAGA
GGAGCUUCACCAGCACACAGAGCAGCUUGUACAGGAUCAAUAUGGAUGUU
AUGUAAUCCAACAUGUACUGGAGCACGGUCGUCCUGAGGAUAAAAGCAAA
AUUGUAGCAGAAAUCCGAGGCAAUGUACUUGUAUUGAGUCAGCACAAAUU
UGCAUGCAAUGUUGUGCAGAAGUGUGUUACUCACGCCUCACGUACGGAGC
GCGCUGUGCUCAUCGAUGAGGUGUGCACCAUGAACGACGGUCCCCACAGU
GCCUUAUACACCAUGAUGAAGGACCAGUAUGCCAGCUACGUGGUCCGCAA
GAUGAUUGACGUGGCGGAGCCAGGCCAGCGGAAGAUCGUCAUGCAUAAGA
UCCGACCCCACAUCGCAACUCUUCGUAAGUACACCUAUGGCAAGCACAUUC
UGGCCAAGCUGGAGAAGUACUACAUGAAGAACGGUGUUGACUUAGGGGGA
GGUGGCGGAUCGGGAGGUGGCGGAUCGGGAGGUGGCGGAUCGGGCCGCAG
CCGCCUUUUGGAAGAUUUUCGAAACAACCGGUACCCCAAUUUACAACUGC
GGGAGAUUGCCGGACAUAUAAUGGAAUUUUCCCAAGACCAGCAUGGGAAC
AGAUUCAUUCAGCUGAAACUGGAGCGUGCCACACCAGCUGAGCGCCAGCU
UGUCUUUAAUGAAAUCCUCCAGGCUGCCUACCAACUCAUGGUGGAUGUGU
UUGGUUGUUACGUCAUUCAGAAGUUCUUUGAAUUUGGCAGUCUUGAACAG
AAGCUGGCUUUGGCAGAACGGAUUCGAGGUCACGUCCUGUCAUUGGCACU
ACAGAUGUAUGGCUCCCGUGUUAUCGAGAAAGCUCUUGAGUUUAUUCCUU
CAGACCAGCAGAAUGAGAUGGUUCGGGAACUAGAUGGCCAUGUCUUGAAG
UGUGUGAAAGAUCAGAAUGGCAAUCACGUGGUUCAGAAAUGCAUUGAAUG
UGUACAGCCCCAGUCUUUGCAAUUUAUCAUCGAUGCGUUUAAGGGACAGG
UAUUUGCCUUAUCCACACAUCCUUAUGGCUGCCGAGUGAUUCAGAGAAUC
CUGGAGCACUGUCUCCCUGACCAGACACUCCCUAUUUUAGAGGAGCUUCAC
CAGCACACAGAGCAGCUUGUACAGGAUCAAUAUGGAAGUUAUGUAAUCCG
CCAUGUACUGGAGCACGGUCGUCCUGAGGAUAAAAGCAAAAUUGUAGCAG
AAAUCCGAGGCAAUGUACUUGUAUUGAGUCAGCACAAAUUUGCAAACAAU
GUUGUGCAGAAGUGUGUUACUCACGCCUCACGUACGGAGCGCGCUGUGCU
CAUCGAUGAGGUGUGCACCAUGAACGACGGUCCCCACAGUGCCUUAUACAC
CAUGAUGAAGGACCAGUAUGCCUGCUACGUGGUCCAGAAGAUGAUUGACG
UGGCGGAGCCAGGCCAGCGGAAGAUCGUCAUGCAUAAGAUCCGACCCCACA
UCGCAACUCUUCGUAAGUACACCUAUGGCAAGCACAUUCUGGCCAAGCUG
GAGAAGUACUACAUGAAGAACGGUGUUGACUUAGGGuga
Protein sequence:
(SEQ ID NO: 16)
MGRSRLLEDFRNNRYPNLQLREIAGHIMEFSQDQHGSRFIRLKLERATPAERQLV
FNEILQAAYQLMVDVFGSYVIEKFFEFGSLEQKLALAERIRGHVLSLALQMYGCR
VIQKALEFIPSDQQNEMVRELDGHVLKCVKDQNGCHVVQKCIECVQPQSLQFIID
AFKGQVFALSTHPYGSRVIERILEHCLPDQTLPILEELHQHTEQLVQDQYGCYVIQ
HVLEHGRPEDKSKIVAEIRGNVLVLSQHKFACNVVQKCVTHASRTERAVLIDEV
CTMNDGPHSALYTMMKDQYASYVVRKMIDVAEPGQRKIVMHKIRPHIATLRKY
TYGKHILAKLEKYYMKNGVDLGGGGGSGGGGSGGGGSGRSRLLEDFRNNRYPN
LQLREIAGHIMEFSQDQHGNRFIQLKLERATPAERQLVFNEILQAAYQLMVDVFG
CYVIQKFFEFGSLEQKLALAERIRGHVLSLALQMYGSRVIEKALEFIPSDQQNEM
VRELDGHVLKCVKDQNGNHVVQKCIECVQPQSLQFIIDAFKGQVFALSTHPYGC
RVIQRILEHCLPDQTLPILEELHQHTEQLVQDQYGSYVIRHVLEHGRPEDKSKIVA
EIRGNVLVLSQHKFANNVVQKCVTHASRTERAVLIDEVCTMNDGPHSALYTMM
KDQYACYVVQKMIDVAEPGQRKIVMHKIRPHIATLRKYTYGKHILAKLEKYYM
KNGVDLG*

Fusion polypeptide of Dual PUF design fused to ADAR2DD (PUF targeted towards nucleotides 1537-1552 of the human GluA2 (Reference sequence NM_000826) nucleotide sequence [aucaugaucaagaagc] (SEQ ID NO: 22))

mRNA sequence:
(SEQ ID NO: 8)
AUGGACUAUAAGGACCACGACGGAGACUACAAGGAUCAUGAUAUUGAUUA
CAAAGACGAUGACGAUAAGAUGGCCCCAAAGAAGAAGCGGAAGGUCGGUA
UCCACGGAGUCCCAGCAGCCCUCCACCUCGACCAAACACCCAGCAGACAGC
CUAUCCCUUCCGAAGGACUGcagcugcauuuaccgcagguuuuagcugacgcugucucacgccugg
uccuggguaaguuuggugaucugaccgacaacuucuccuccccucacgcucgcagaaaagugcuggcuggagucgu
caugacaacaggcacagauguuaaagaugccaaggugauaaguguuucuacaggaggcaaauguauuaauggugaa
uacaugagugaucguggccuugcauuaaaugacugccaugcagaaauaauaucucggagauccuugcucagauuuc
uuuauacacaacuugagcuuuacuuaaauaacaaagaugaucaaaaaagauccaucuuucagaaaucagagcgaggg
ggguuuaggcugaaggagaauguccaguuucaucuguacaucagcaccucucccuguggagaugccagaaucuuc
ucaccacaugagccaauccuggaagaaccagcagauagacacccaaaucguaaagcaagaggacagcuacggaccaaa
auagagucuggucaggggacgauuccagugcgcuccaaugcgagcauccaaacgugggacggggugcugcaaggg
gagcggcugcucaccauguccugcagugacaagauugcacgcuggaacguggugggcauccagggaucacugcuca
gcauuuucguggagcccauuuacuucucgagcaucauccugggcagccuuuaccacggggaccaccuuuccagggc
cauguaccagcggaucuccaacauagaggaccugccaccucucuacacccucaacaagccuuugcucaguggcauca
gcaaugcagaagcacggcagccagggaaggcccccaacuucagugucaacuggacgguaggcgacuccgcuauuga
ggucaucaacgccacgacugggaaggaugagcugggccgcgcgucccgccuguguaagcacgcguuguacugucgc
uggaugcgugugcacggcaagguucccucccacuuacuacgcuccaagauuaccaagcccaacguguaccaugagu
ccaagcuggcggcaaaggaguaccaggccgccaaggcgcgucuguucacagccuucaucaaggcggggcugggggc
cuggguggagaagcccaccgagcaggaccaguucucacucacgCCUGGAGGUGGCGGAUCGGGAG
GUGGCGGAUCGGGAGGUGGCGGAUCGGGCCGCAGCCGCCUUUUGGAAGAU
UUUCGAAACAACCGGUACCCCAAUUUACAACUGCGGGAGAUUGCCGGACA
UAUAAUGGAAUUUUCCCAAGACCAGCAUGGGUCCAGAUUCAUUCGCCUGA
AACUGGAGCGUGCCACACCAGCUGAGCGCCAGCUUGUCUUUAAUGAAAUC
CUCCAGGCUGCCUACCAACUCAUGGUGGAUGUGUUUGGUAGUUACGUCAU
UGAGAAGUUCUUUGAAUUUGGCAGUCUUGAACAGAAGCUGGCUUUGGCAG
AACGGAUUCGAGGUCACGUCCUGUCAUUGGCACUACAGAUGUAUGGCUGC
CGUGUUAUCCAGAAAGCUCUUGAGUUUAUUCCUUCAGACCAGCAGAAUGA
GAUGGUUCGGGAACUAGAUGGCCAUGUCUUGAAGUGUGUGAAAGAUCAGA
AUGGCUGUCACGUGGUUCAGAAAUGCAUUGAAUGUGUACAGCCCCAGUCU
UUGCAAUUUAUCAUCGAUGCGUUUAAGGGCCAGGUAUUUGCCUUAUCCAC
ACAUCCUUAUGGCUCCCGAGUGAUUGAGAGAAUCCUGGAGCACUGUCUCC
CUGACCAGACACUCCCUAUUUUAGAGGAGCUUCACCAGCACACAGAGCAGC
UUGUACAGGAUCAAUAUGGAUGUUAUGUAAUCCAACAUGUACUGGAGCAC
GGUCGUCCUGAGGAUAAAAGCAAAAUUGUAGCAGAAAUCCGAGGCAAUGU
ACUUGUAUUGAGUCAGCACAAAUUUGCAUGCAAUGUUGUGCAGAAGUGUG
UUACUCACGCCUCACGUACGGAGCGCGCUGUGCUCAUCGAUGAGGUGUGC
ACCAUGAACGACGGUCCCCACAGUGCCUUAUACACCAUGAUGAAGGACCAG
UAUGCCAGCUACGUGGUCCGCAAGAUGAUUGACGUGGCGGAGCCAGGCCA
GCGGAAGAUCGUCAUGCAUAAGAUCCGACCCCACAUCGCAACUCUUCGUAA
GUACACCUAUGGCAAGCACAUUCUGGCCAAGCUGGAGAAGUACUACAUGA
AGAACGGUGUUGACUUAGGGGGAGGUGGCGGAUCGGGAGGUGGCGGAUCG
GGAGGUGGCGGAUCGGGCCGCAGCCGCCUUUUGGAAGAUUUUCGAAACAA
CCGGUACCCCAAUUUACAACUGCGGGAGAUUGCCGGACAUAUAAUGGAAU
UUUCCCAAGACCAGCAUGGGAACAGAUUCAUUCAGCUGAAACUGGAGCGU
GCCACACCAGCUGAGCGCCAGCUUGUCUUUAAUGAAAUCCUCCAGGCUGCC
UACCAACUCAUGGUGGAUGUGUUUGGUUGUUACGUCAUUCAGAAGUUCUU
UGAAUUUGGCAGUCUUGAACAGAAGCUGGCUUUGGCAGAACGGAUUCGAG
GUCACGUCCUGUCAUUGGCACUACAGAUGUAUGGCUCCCGUGUUAUCGAG
AAAGCUCUUGAGUUUAUUCCUUCAGACCAGCAGAAUGAGAUGGUUCGGGA
ACUAGAUGGCCAUGUCUUGAAGUGUGUGAAAGAUCAGAAUGGCAAUCACG
UGGUUCAGAAAUGCAUUGAAUGUGUACAGCCCCAGUCUUUGCAAUUUAUC
AUCGAUGCGUUUAAGGGACAGGUAUUUGCCUUAUCCACACAUCCUUAUGG
CUGCCGAGUGAUUCAGAGAAUCCUGGAGCACUGUCUCCCUGACCAGACACU
CCCUAUUUUAGAGGAGCUUCACCAGCACACAGAGCAGCUUGUACAGGAUC
AAUAUGGAAGUUAUGUAAUCCGCCAUGUACUGGAGCACGGUCGUCCUGAG
GAUAAAAGCAAAAUUGUAGCAGAAAUCCGAGGCAAUGUACUUGUAUUGAG
UCAGCACAAAUUUGCAAACAAUGUUGUGCAGAAGUGUGUUACUCACGCCU
CACGUACGGAGCGCGCUGUGCUCAUCGAUGAGGUGUGCACCAUGAACGAC
GGUCCCCACAGUGCCUUAUACACCAUGAUGAAGGACCAGUAUGCCUGCUAC
GUGGUCCAGAAGAUGAUUGACGUGGCGGAGCCAGGCCAGCGGAAGAUCGU
CAUGCAUAAGAUCCGACCCCACAUCGCAACUCUUCGUAAGUACACCUAUGG
CAAGCACAUUCUGGCCAAGCUGGAGAAGUACUACAUGAAGAACGGUGUUG
ACUUAGGGAGCGGCGGCAAGCGGCCCGCCGCCACCAAGAAGGCCGGCCAGG
CCAAGAAGAAGAAGuga
Protein sequence:
(SEQ ID NO: 17)
MDYKDHDGDYKDHDIDYKDDDDKMAPKKKRKVGIHGVPAALHLDQTPSRQPI
PSEGLQLHLPQVLADAVSRLVLGKFGDLTDNFSSPHARRKVLAGVVMTTGTDV
KDAKVISVSTGGKCINGEYMSDRGLALNDCHAEIISRRSLLRFLYTQLELYLNNK
DDQKRSIFQKSERGGFRLKENVQFHLYISTSPCGDARIFSPHEPILEEPADRHPNRK
ARGQLRTKIESGQGTIPVRSNASIQTWDGVLQGERLLTMSCSDKIARWNVVGIQG
SLLSIFVEPIYFSSIILGSLYHGDHLSRAMYQRISNIEDLPPLYTLNKPLLSGISNAEA
RQPGKAPNFSVNWTVGDSAIEVINATTGKDELGRASRLCKHALYCRWMRVHGK
VPSHLLRSKITKPNVYHESKLAAKEYQAAKARLFTAFIKAGLGAWVEKPTEQDQ
FSLTPGGGGSGGGGSGGGGSGRSRLLEDFRNNRYPNLQLREIAGHIMEFSQDQH
GSRFIRLKLERATPAERQLVFNEILQAAYQLMVDVFGSYVIEKFFEFGSLEQKLAL
AERIRGHVLSLALQMYGCRVIQKALEFIPSDQQNEMVRELDGHVLKCVKDQNGC
HVVQKCIECVQPQSLQFIIDAFKGQVFALSTHPYGSRVIERILEHCLPDQTLPILEE
LHQHTEQLVQDQYGCYVIQHVLEHGRPEDKSKIVAEIRGNVLVLSQHKFACNVV
QKCVTHASRTERAVLIDEVCTMNDGPHSALYTMMKDQYASYVVRKMIDVAEP
GQRKIVMHKIRPHIATLRKYTYGKHILAKLEKYYMKNGVDLGGGGGSGGGGSG
GGGSGRSRLLEDFRNNRYPNLQLREIAGHIMEFSQDQHGNRFIQLKLERATPAER
QLVFNEILQAAYQLMVDVFGCYVIQKFFEFGSLEQKLALAERIRGHVLSLALQM
YGSRVIEKALEFIPSDQQNEMVRELDGHVLKCVKDQNGNHVVQKCIECVQPQSL
QFIIDAFKGQVFALSTHPYGCRVIQRILEHCLPDQTLPILEELHQHTEQLVQDQYG
SYVIRHVLEHGRPEDKSKIVAEIRGNVLVLSQHKFANNVVQKCVTHASRTERAV
LIDEVCTMNDGPHSALYTMMKDQYACYVVQKMIDVAEPGQRKIVMHKIRPHIA
TLRKYTYGKHILAKLEKYYMKNGVDLGSGGKRPAATKKAGQAKKKK*

Dual PUF design with (G4S)3 linker targeted towards nucleotides 31,995-32,010 of the human SMN2 (Reference sequence NM-022876) nucleotide sequence (ACAGGGUUUUAGACAA (SEQ ID NO: 24))

mRNA sequence:
(SEQ ID NO: 9)
augGGCCGCAGCCGCCUUUUGGAAGAUUUUCGAAACAACCGGUACCCCAAU
UUACAACUGCGGGAGAUUGCCGGACAUAUAAUGGAAUUUUCCCAAGACCA
GCAUGGGUCCAGAUUCAUUCAGCUGAAACUGGAGCGUGCCACACCAGCUG
AGCGCCAGCUUGUCUUUAAUGAAAUCCUCCAGGCUGCCUACCAACUCAUGG
UGGAUGUGUUUGGUUGUUACGUCAUUCAGAAGUUCUUUGAAUUUGGCAGU
CUUGAACAGAAGCUGGCUUUGGCAGAACGGAUUCGAGGUCACGUCCUGUC
AUUGGCACUACAGAUGUAUGGCUCCCGUGUUAUCCGCAAAGCUCUUGAGU
UUAUUCCUUCAGACCAGCAGAAUGAGAUGGUUCGGGAACUAGAUGGCCAU
GUCUUGAAGUGUGUGAAAGAUCAGAAUGGCUGUCACGUGGUUCAGAAAUG
CAUUGAAUGUGUACAGCCCCAGUCUUUGCAAUUUAUCAUCGAUGCGUUUA
AGGGCCAGGUAUUUGCCUUAUCCACACAUCCUUAUGGCUCCCGAGUGAUU
GAGAGAAUCCUGGAGCACUGUCUCCCUGACCAGACACUCCCUAUUUUAGA
GGAGCUUCACCAGCACACAGAGCAGCUUGUACAGGAUCAAUAUGGAUGUU
AUGUAAUCCAACAUGUACUGGAGCACGGUCGUCCUGAGGAUAAAAGCAAA
AUUGUAGCAGAAAUCCGAGGCAAUGUACUUGUAUUGAGUCAGCACAAAUU
UGCAAACAAUGUUGUGCAGAAGUGUGUUACUCACGCCUCACGUACGGAGC
GCGCUGUGCUCAUCGAUGAGGUGUGCACCAUGAACGACGGUCCCCACAGU
GCCUUAUACACCAUGAUGAAGGACCAGUAUGCCAACUACGUGGUCCAGAA
GAUGAUUGACGUGGCGGAGCCAGGCCAGCGGAAGAUCGUCAUGCAUAAGA
UCCGACCCCACAUCGCAACUCUUCGUAAGUACACCUAUGGCAAGCACAUUC
UGGCCAAGCUGGAGAAGUACUACAUGAAGAACGGUGUUGACUUAGGGGGA
GGUGGCGGAUCGGGAGGUGGCGGAUCGGGAGGUGGCGGAUCGGGCCGCAG
CCGCCUUUUGGAAGAUUUUCGAAACAACCGGUACCCCAAUUUACAACUGC
GGGAGAUUGCCGGACAUAUAAUGGAAUUUUCCCAAGACCAGCAUGGGAAC
AGAUUCAUUCAGCUGAAACUGGAGCGUGCCACACCAGCUGAGCGCCAGCU
UGUCUUUAAUGAAAUCCUCCAGGCUGCCUACCAACUCAUGGUGGAUGUGU
UUGGUAAUUACGUCAUUCAGAAGUUCUUUGAAUUUGGCAGUCUUGAACAG
AAGCUGGCUUUGGCAGAACGGAUUCGAGGUCACGUCCUGUCAUUGGCACU
ACAGAUGUAUGGCUCCCGUGUUAUCGAGAAAGCUCUUGAGUUUAUUCCUU
CAGACCAGCAGAAUGAGAUGGUUCGGGAACUAGAUGGCCAUGUCUUGAAG
UGUGUGAAAGAUCAGAAUGGCAGUCACGUGGUUGAGAAAUGCAUUGAAUG
UGUACAGCCCCAGUCUUUGCAAUUUAUCAUCGAUGCGUUUAAGGGACAGG
UAUUUGCCUUAUCCACACAUCCUUAUGGCUCCCGAGUGAUUGAGAGAAUC
CUGGAGCACUGUCUCCCUGACCAGACACUCCCUAUUUUAGAGGAGCUUCAC
CAGCACACAGAGCAGCUUGUACAGGAUCAAUAUGGAUGUUAUGUAAUCCA
ACAUGUACUGGAGCACGGUCGUCCUGAGGAUAAAAGCAAAAUUGUAGCAG
AAAUCCGAGGCAAUGUACUUGUAUUGAGUCAGCACAAAUUUGCAAGCUAU
GUUGUGCGCAAGUGUGUUACUCACGCCUCACGUACGGAGCGCGCUGUGCU
CAUCGAUGAGGUGUGCACCAUGAACGACGGUCCCCACAGUGCCUUAUACAC
CAUGAUGAAGGACCAGUAUGCCUGCUACGUGGUCCAGAAGAUGAUUGACG
UGGCGGAGCCAGGCCAGCGGAAGAUCGUCAUGCAUAAGAUCCGACCCCACA
UCGCAACUCUUCGUAAGUACACCUAUGGCAAGCACAUUCUGGCCAAGCUG
GAGAAGUACUACAUGAAGAACGGUGUUGACUUAGGGuga
Protein sequence:
(SEQ ID NO: 18)
MGRSRLLEDFRNNRYPNLQLREIAGHIMEFSQDQHGSRFIQLKLERATPAERQLV
FNEILQAAYQLMVDVFGCYVIQKFFEFGSLEQKLALAERIRGHVLSLALQMYGS
RVIRKALEFIPSDQQNEMVRELDGHVLKCVKDQNGCHVVQKCIECVQPQSLQFII
DAFKGQVFALSTHPYGSRVIERILEHCLPDQTLPILEELHQHTEQLVQDQYGCYVI
QHVLEHGRPEDKSKIVAEIRGNVLVLSQHKFANNVVQKCVTHASRTERAVLIDE
VCTMNDGPHSALYTMMKDQYANYVVQKMIDVAEPGQRKIVMHKIRPHIATLR
KYTYGKHILAKLEKYYMKNGVDLGGGGGSGGGGSGGGGSGRSRLLEDFRNNR
YPNLQLREIAGHIMEFSQDQHGNRFIQLKLERATPAERQLVFNEILQAAYQLMVD
VFGNYVIQKFFEFGSLEQKLALAERIRGHVLSLALQMYGSRVIEKALEFIPSDQQN
EMVRELDGHVLKCVKDQNGSHVVEKCIECVQPQSLQFIIDAFKGQVFALSTHPY
GSRVIERILEHCLPDQTLPILEELHQHTEQLVQDQYGCYVIQHVLEHGRPEDKSKI
VAEIRGNVLVLSQHKFASYVVRKCVTHASRTERAVLIDEVCTMNDGPHSALYT
MMKDQYACYVVQKMIDVAEPGQRKIVMHKIRPHIATLRKYTYGKHILAKLEKY
YMKNGVDLG*

Fusion polypeptide of Dual PUF design fused to ADAR2DD (PUF targeted towards nucleotides 31,995-32,010 of the human SMN2 (Reference sequence NM-022876) nucleotide sequence [ACAGGGUUUUAGACAA] (SEQ ID NO: 25))

mRNA sequence:
(SEQ ID NO: 10)
AUGGACUAUAAGGACCACGACGGAGACUACAAGGAUCAUGAUAUUGAUUA
CAAAGACGAUGACGAUAAGAUGGCCCCAAAGAAGAAGCGGAAGGUCGGUA
UCCACGGAGUCCCAGCAGCCCUCCACCUCGACCAAACACCCAGCAGACAGC
CUAUCCCUUCCGAAGGACUGcagcugcauuuaccgcagguuuuagcugacgcugucucacgccugg
uccuggguaaguuuggugaucugaccgacaacuucuccuccccucacgcucgcagaaaagugcuggcuggagucgu
caugacaacaggcacagauguuaaagaugccaaggugauaaguguuucuacaggaggcaaauguauuaauggugaa
uacaugagugaucguggccuugcauuaaaugacugccaugcagaaauaauaucucggagauccuugcucagauuuc
uuuauacacaacuugagcuuuacuuaaauaacaaagaugaucaaaaaagauccaucuuucagaaaucagagcgaggg
ggguuuaggcugaaggagaauguccaguuucaucuguacaucagcaccucucccuguggagaugccagaaucuuc
ucaccacaugagccaauccuggaagaaccagcagauagacacccaaaucguaaagcaagaggacagcuacggaccaaa
auagagucuggucaggggacgauuccagugcgcuccaaugcgagcauccaaacgugggacggggugcugcaaggg
gagcggcugcucaccauguccugcagugacaagauugcacgcuggaacguggugggcauccagggaucacugcuca
gcauuuucguggagcccauuuacuucucgagcaucauccugggcagccuuuaccacggggaccaccuuuccagggc
cauguaccagcggaucuccaacauagaggaccugccaccucucuacacccucaacaagccuuugcucaguggcauca
gcaaugcagaagcacggcagccagggaaggcccccaacuucagugucaacuggacgguaggcgacuccgcuauuga
ggucaucaacgccacgacugggaaggaugagcugggccgcgcgucccgccuguguaagcacgcguuguacugucgc
uggaugcgugugcacggcaagguucccucccacuuacuacgcuccaagauuaccaagcccaacguguaccaugagu
ccaagcuggcggcaaaggaguaccaggccgccaaggcgcgucuguucacagccuucaucaaggcggggcugggggc
cuggguggagaagcccaccgagcaggaccaguucucacucacgCCUGGAGGUGGCGGAUCGGGAG
GUGGCGGAUCGGGAGGUGGCGGAUCGGGCCGCAGCCGCCUUUUGGAAGAU
UUUCGAAACAACCGGUACCCCAAUUUACAACUGCGGGAGAUUGCCGGACA
UAUAAUGGAAUUUUCCCAAGACCAGCAUGGGUCCAGAUUCAUUCAGCUGA
AACUGGAGCGUGCCACACCAGCUGAGCGCCAGCUUGUCUUUAAUGAAAUC
CUCCAGGCUGCCUACCAACUCAUGGUGGAUGUGUUUGGUUGUUACGUCAU
UCAGAAGUUCUUUGAAUUUGGCAGUCUUGAACAGAAGCUGGCUUUGGCAG
AACGGAUUCGAGGUCACGUCCUGUCAUUGGCACUACAGAUGUAUGGCUCC
CGUGUUAUCCGCAAAGCUCUUGAGUUUAUUCCUUCAGACCAGCAGAAUGA
GAUGGUUCGGGAACUAGAUGGCCAUGUCUUGAAGUGUGUGAAAGAUCAGA
AUGGCUGUCACGUGGUUCAGAAAUGCAUUGAAUGUGUACAGCCCCAGUCU
UUGCAAUUUAUCAUCGAUGCGUUUAAGGGCCAGGUAUUUGCCUUAUCCAC
ACAUCCUUAUGGCUCCCGAGUGAUUGAGAGAAUCCUGGAGCACUGUCUCC
CUGACCAGACACUCCCUAUUUUAGAGGAGCUUCACCAGCACACAGAGCAGC
UUGUACAGGAUCAAUAUGGAUGUUAUGUAAUCCAACAUGUACUGGAGCAC
GGUCGUCCUGAGGAUAAAAGCAAAAUUGUAGCAGAAAUCCGAGGCAAUGU
ACUUGUAUUGAGUCAGCACAAAUUUGCAAACAAUGUUGUGCAGAAGUGUG
UUACUCACGCCUCACGUACGGAGCGCGCUGUGCUCAUCGAUGAGGUGUGC
ACCAUGAACGACGGUCCCCACAGUGCCUUAUACACCAUGAUGAAGGACCAG
UAUGCCAACUACGUGGUCCAGAAGAUGAUUGACGUGGCGGAGCCAGGCCA
GCGGAAGAUCGUCAUGCAUAAGAUCCGACCCCACAUCGCAACUCUUCGUAA
GUACACCUAUGGCAAGCACAUUCUGGCCAAGCUGGAGAAGUACUACAUGA
AGAACGGUGUUGACUUAGGGGGAGGUGGCGGAUCGGGAGGUGGCGGAUCG
GGAGGUGGCGGAUCGGGCCGCAGCCGCCUUUUGGAAGAUUUUCGAAACAA
CCGGUACCCCAAUUUACAACUGCGGGAGAUUGCCGGACAUAUAAUGGAAU
UUUCCCAAGACCAGCAUGGGAACAGAUUCAUUCAGCUGAAACUGGAGCGU
GCCACACCAGCUGAGCGCCAGCUUGUCUUUAAUGAAAUCCUCCAGGCUGCC
UACCAACUCAUGGUGGAUGUGUUUGGUAAUUACGUCAUUCAGAAGUUCUU
UGAAUUUGGCAGUCUUGAACAGAAGCUGGCUUUGGCAGAACGGAUUCGAG
GUCACGUCCUGUCAUUGGCACUACAGAUGUAUGGCUCCCGUGUUAUCGAG
AAAGCUCUUGAGUUUAUUCCUUCAGACCAGCAGAAUGAGAUGGUUCGGGA
ACUAGAUGGCCAUGUCUUGAAGUGUGUGAAAGAUCAGAAUGGCAGUCACG
UGGUUGAGAAAUGCAUUGAAUGUGUACAGCCCCAGUCUUUGCAAUUUAUC
AUCGAUGCGUUUAAGGGACAGGUAUUUGCCUUAUCCACACAUCCUUAUGG
CUCCCGAGUGAUUGAGAGAAUCCUGGAGCACUGUCUCCCUGACCAGACACU
CCCUAUUUUAGAGGAGCUUCACCAGCACACAGAGCAGCUUGUACAGGAUC
AAUAUGGAUGUUAUGUAAUCCAACAUGUACUGGAGCACGGUCGUCCUGAG
GAUAAAAGCAAAAUUGUAGCAGAAAUCCGAGGCAAUGUACUUGUAUUGAG
UCAGCACAAAUUUGCAAGCUAUGUUGUGCGCAAGUGUGUUACUCACGCCU
CACGUACGGAGCGCGCUGUGCUCAUCGAUGAGGUGUGCACCAUGAACGAC
GGUCCCCACAGUGCCUUAUACACCAUGAUGAAGGACCAGUAUGCCUGCUAC
GUGGUCCAGAAGAUGAUUGACGUGGCGGAGCCAGGCCAGCGGAAGAUCGU
CAUGCAUAAGAUCCGACCCCACAUCGCAACUCUUCGUAAGUACACCUAUGG
CAAGCACAUUCUGGCCAAGCUGGAGAAGUACUACAUGAAGAACGGUGUUG
ACUUAGGGAGCGGCGGCAAGCGGCCCGCCGCCACCAAGAAGGCCGGCCAGG
CCAAGAAGAAGAAGuga
Protein sequence:
(SEQ ID NO: 19)
MDYKDHDGDYKDHDIDYKDDDDKMAPKKKRKVGIHGVPAALHLDQTPSRQPI
PSEGLQLHLPQVLADAVSRLVLGKFGDLTDNFSSPHARRKVLAGVVMTTGTDV
KDAKVISVSTGGKCINGEYMSDRGLALNDCHAEIISRRSLLRFLYTQLELYLNNK
DDQKRSIFQKSERGGFRLKENVQFHLYISTSPCGDARIFSPHEPILEEPADRHPNRK
ARGQLRTKIESGQGTIPVRSNASIQTWDGVLQGERLLTMSCSDKIARWNVVGIQG
SLLSIFVEPIYFSSIILGSLYHGDHLSRAMYQRISNIEDLPPLYTLNKPLLSGISNAEA
RQPGKAPNFSVNWTVGDSAIEVINATTGKDELGRASRLCKHALYCRWMRVHGK
VPSHLLRSKITKPNVYHESKLAAKEYQAAKARLFTAFIKAGLGAWVEKPTEQDQ
FSLTPGGGGSGGGGSGGGGSGRSRLLEDFRNNRYPNLQLREIAGHIMEFSQDQH
GSRFIQLKLERATPAERQLVFNEILQAAYQLMVDVFGCYVIQKFFEFGSLEQKLA
LAERIRGHVLSLALQMYGSRVIRKALEFIPSDQQNEMVRELDGHVLKCVKDQNG
CHVVQKCIECVQPQSLQFIIDAFKGQVFALSTHPYGSRVIERILEHCLPDQTLPILE
ELHQHTEQLVQDQYGCYVIQHVLEHGRPEDKSKIVAEIRGNVLVLSQHKFANNV
VQKCVTHASRTERAVLIDEVCTMNDGPHSALYTMMKDQYANYVVQKMIDVAE
PGQRKIVMHKIRPHIATLRKYTYGKHILAKLEKYYMKNGVDLGGGGGSGGGGS
GGGGSGRSRLLEDFRNNRYPNLQLREIAGHIMEFSQDQHGNRFIQLKLERATPAE
RQLVFNEILQAAYQLMVDVFGNYVIQKFFEFGSLEQKLALAERIRGHVLSLALQ
MYGSRVIEKALEFIPSDQQNEMVRELDGHVLKCVKDQNGSHVVEKCIECVQPQS
LQFIIDAFKGQVFALSTHPYGSRVIERILEHCLPDQTLPILEELHQHTEQLVQDQYG
CYVIQHVLEHGRPEDKSKIVAEIRGNVLVLSQHKFASYVVRKCVTHASRTERAV
LIDEVCTMNDGPHSALYTMMKDQYACYVVQKMIDVAEPGQRKIVMHKIRPHIA
TLRKYTYGKHILAKLEKYYMKNGVDLGSGGKRPAATKKAGQAKKKK*
Splicing modulator hTRA2-beta1
mRNA sequence (PUF insertion site (e.g., deletion site) in underlined bold region
[BbsI cassette]):
(SEQ ID NO: 11)
AUGGACUACAAGGACCACGAUGGAGAUUAUAAAGACCACGACAUCGACUA
UAAGGACGACGACGACAAGAUGAGCGACAGCGGCGAGCAGAACUACGGCG
AGAGAGAGUCCAGAAGCGCCAGCAGAUCCGGCUCCGCUCACGGAAGCGGA
AAGAGCGCUAGACAUACCCCCGCCAGAAGCAGAUCCAAGGAGGAUUCUAG
AAGGAGCAGAAGCAAGAGCAGAUCUAGAAGCGAAUCUAGAUCCAGAUCUA
GAAGAAGCUCUAGAAGGCACUACACAAGGUCUAGAAGCAGAUCUAGAAGC
CAUAGAAGAAGCAGAUCCAGAAGCUACUCUAGAGACUACAGAAGGAGACA
CAGCCACUCCCACAGCCCUAUGUCCACAAGAAGAAGGCACGUGGGCAAUAG
GGCCAACCCCGACCCUAACCCCAAGAAGAAGAGGAAGGUGGGCUCCGGCG
UCUUCucGAAGACGGCAGCGGCCCUAAGAAGAAGAGGAAGGUGGGCAGC
AGCAGCAUCACCAAGAGACCCCACACCCCUACCCCCGGCAUCUACAUGGGC
AGACCCACCUACGGCUCCUCUAGAAGGAGAGACUACUACGACAGAGGCUAC
GAUAGAGGCUACGACGAUAGAGAUUAUUACUCUAGAUCCUACAGAGGCGG
CGGAGGAGGCGGAGGCGGAUGGAGAGCUGCCCAAGACAGAGACCAGAUCU
AUAGAAGAAGGAGCCCCAGCCCCUACUAUAGCAGAGGCGGCUACAGAUCU
AGAUCUAGAUCUAGAAGCUAUAGCCCCAGAAGAUACGGCGGCAGCUACCC
UUACGACGUGCCCGACUACGCCUGA
Protein sequence (PUF inserted in underlined bold region [BbsI cassette]):
(SEQ ID NO: 20)
MDYKDHDGDYKDHDIDYKDDDDKMSDSGEQNYGERESRSASRSGSAHGSGKS
ARHTPARSRSKEDSRRSRSKSRSRSESRSRSRRSSRRHYTRSRSRSRSHRRSRSRS
YSRDYRRRHSHSHSPMSTRRRHVGNRANPDPNPKKKRKVGSGVFGEDGSGPKK
KRKVGSSSITKRPHTPTPGIYMGRPTYGSSRRRDYYDRGYDRGYDDRDYYSRSY
RGGGGGGGGWRAAQDRDQIYRRRSPSPYYSRGGYRSRSRSRSYSPRRYGGSYPY
DVPDYA*
Fusion polypeptide of hTRA2-beta1 with dual PUF design
mRNA sequence:
(SEQ ID NO: 12)
AUGGACUACAAGGACCACGAUGGAGAUUAUAAAGACCACGACAUCGACUA
UAAGGACGACGACGACAAGAUGAGCGACAGCGGCGAGCAGAACUACGGCG
AGAGAGAGUCCAGAAGCGCCAGCAGAUCCGGCUCCGCUCACGGAAGCGGA
AAGAGCGCUAGACAUACCCCCGCCAGAAGCAGAUCCAAGGAGGAUUCUAG
AAGGAGCAGAAGCAAGAGCAGAUCUAGAAGCGAAUCUAGAUCCAGAUCUA
GAAGAAGCUCUAGAAGGCACUACACAAGGUCUAGAAGCAGAUCUAGAAGC
CAUAGAAGAAGCAGAUCCAGAAGCUACUCUAGAGACUACAGAAGGAGACA
CAGCCACUCCCACAGCCCUAUGUCCACAAGAAGAAGGCACGUGGGCAAUAG
GGCCAACCCCGACCCUAACCCCAAGAAGAAGAGGAAGGUGGGCGGAGGUG
GCGGAUCGggcaggagcaggcuuuuggaagauuuucgaaacaaccgCuaccccaauuuacaacugcgggaga
uugcuggacauauaauggaauuuucccaagaccagcauggguccagauucauucagcugaaacuggagcgugccac
accagcugagcgccagcuugucuucaaugaaauccuccaggcugccuaccaacucaugguggauguguuugguaau
uacgucauucagaaguucuuugaauuuggcagucuugaacagaagcuggcuuuggcagaacggauucgaggccac
guccugucauuggcacuacagauguauggcugccguguuauccagaaagcucuugaguuuauuccuucagaccag
cagaaugagaugguucgggaacuagauggccaugucuugaagugugugaaagaucagaauggcaaucacgugguu
cagaaaugcauugaauguguacagccccagucuuugcaauuuaucaucgaugcguuuaagggacagguauuugcc
uuauccacacauccuuauggcugccgagugauucagagaauccuggagcacugucucccugaccagacacucccua
uuuuagaggagcuucaccagcacacagagcagcuuguacaggaucaauauggaaauuauguaauccaacauguacu
ggagcacggucguccugaggauaaaagcaaaauuguagcagaaauccgaggcaauguacuuguauugagucagcac
aaauuugcaagcaauguuguggagaaguguguuacucacgccucacguacggagcgcgcugugcucaucgaugag
gugugcaccaugaacgacgguccccacagugccuuauacaccaugaugaaggaccaguaugccaacuacguggucc
agaagaugauugacguggcggagccaggccagcggaagaucgucaugcauaagauccggccccacaucgcaacucu
ucguaaguacaccuauggcaagcacauucuggccaagcuggagaaguacuacaugaagaacgguguugacuuaggg
GGAGGUGGCGGAUCGGGAGGUGGCGGAUCGGGAGGUGGCGGAUCGggcaggag
caggcuuuuggaagauuuucgaaacaaccgCuaccccaauuuacaacugcgggagauugcuggacauauaauggaa
uuuucccaagaccagcauggguccagauucauucagcugaaacuggagcgugccacaccagcugagcgccagcuug
ucuucaaugaaauccuccaggcugccuaccaacucaugguggauguguuugguaauuacgucauucagaaguucu
uugaauuuggcagucuugaacagaagcuggcuuuggcagaacggauucgaggccacguccugucauuggcacuac
agauguauggcugccguguuauccagaaagcucuugaguuuauuccuucagaccagcagaaugagaugguucggg
aacuagauggccaugucuugaagugugugaaagaucagaauggcaaucacgugguucagaaaugcauugaaugug
uacagccccagucuuugcaauuuaucaucgaugcguuuaagggacagguauuugccuuauccacacauccuuaugg
cugccgagugauucagagaauccuggagcacugucucccugaccagacacucccuauuuuagaggagcuucaccag
cacacagagcagcuuguacaggaucaauauggaaauuauguaauccaacauguacuggagcacggucguccugagg
auaaaagcaaaauuguagcagaaauccgaggcaauguacuuguauugagucagcacaaauuugcaagcaauguugu
ggagaaguguguuacucacgccucacguacggagcgcgcugugcucaucgaugaggugugcaccaugaacgacgg
uccccacagugccuuauacaccaugaugaaggaccaguaugccaacuacgugguccagaagaugauugacguggcg
gagccaggccagcggaagaucgucaugcauaagauccggccccacaucgcaacucuucguaaguacaccuauggcaa
gcacauucuggccaagcuggagaaguacuacaugaagaacgguguugacuuagggAGCGGCGGCGGCCC
UAAGAAGAAGAGGAAGGUGGGCAGCAGCAGCAUCACCAAGAGACCCCACA
CCCCUACCCCCGGCAUCUACAUGGGCAGACCCACCUACGGCUCCUCUAGAA
GGAGAGACUACUACGACAGAGGCUACGAUAGAGGCUACGACGAUAGAGAU
UAUUACUCUAGAUCCUACAGAGGCGGCGGAGGAGGCGGAGGCGGAUGGAG
AGCUGCCCAAGACAGAGACCAGAUCUAUAGAAGAAGGAGCCCCAGCCCCUA
CUAUAGCAGAGGCGGCUACAGAUCUAGAUCUAGAUCUAGAAGCUAUAGCC
CCAGAAGAUACGGCGGCAGCUACCCUUACGACGUGCCCGACUACGCCUGA
Protein sequence:
(SEQ ID NO: 21)
MDYKDHDGDYKDHDIDYKDDDDKMSDSGEQNYGERESRSASRSGSAHGSGKS
ARHTPARSRSKEDSRRSRSKSRSRSESRSRSRRSSRRHYTRSRSRSRSHRRSRSRS
YSRDYRRRHSHSHSPMSTRRRHVGNRANPDPNPKKKRKVGGGGGSGRSRLLED
FRNNRYPNLQLREIAGHIMEFSQDQHGSRFIQLKLERATPAERQLVFNEILQAAY
QLMVDVFGNYVIQKFFEFGSLEQKLALAERIRGHVLSLALQMYGCRVIQKALEFI
PSDQQNEMVRELDGHVLKCVKDQNGNHVVQKCIECVQPQSLQFIIDAFKGQVFA
LSTHPYGCRVIQRILEHCLPDQTLPILEELHQHTEQLVQDQYGNYVIQHVLEHGRP
EDKSKIVAEIRGNVLVLSQHKFASNVVEKCVTHASRTERAVLIDEVCTMNDGPH
SALYTMMKDQYANYVVQKMIDVAEPGQRKIVMHKIRPHIATLRKYTYGKHILA
KLEKYYMKNGVDLGGGGGSGGGGSGGGGSGRSRLLEDFRNNRYPNLQLREIAG
HIMEFSQDQHGSRFIQLKLERATPAERQLVFNEILQAAYQLMVDVFGNYVIQKFF
EFGSLEQKLALAERIRGHVLSLALQMYGCRVIQKALEFIPSDQQNEMVRELDGH
VLKCVKDQNGNHVVQKCIECVQPQSLQFIIDAFKGQVFALSTHPYGCRVIQRILE
HCLPDQTLPILEELHQHTEQLVQDQYGNYVIQHVLEHGRPEDKSKIVAEIRGNVL
VLSQHKFASNVVEKCVTHASRTERAVLIDEVCTMNDGPHSALYTMMKDQYAN
YVVQKMIDVAEPGQRKIVMHKIRPHIATLRKYTYGKHILAKLEKYYMKNGVDL
GSGGGPKKKRKVGSSSITKRPHTPTPGIYMGRPTYGSSRRRDYYDRGYDRGYDD
RDYYSRSYRGGGGGGGGWRAAQDRDQIYRRRSPSPYYSRGGYRSRSRSRSYSPR
RYGGSYPYDVPDYA*

### RNA Effector

In some embodiments, a polypeptide described herein comprises an RNA effector.

In some embodiments, the RNA effector does not comprise nuclease (e.g., endonuclease and/or exonuclease) activity. In some embodiments, the RNA effector does not comprise a nuclease (e.g., an endonuclease and/or exonuclease). In some embodiments, the RNA effector does not comprise a nuclease or a functional fragment thereof. In some embodiments, an RNA effector does not break a phosphodiester bond.

A further example of an RNA effector is a splicing modulator, e.g., a splicing factor. A splicing modulator can include an agent that recruit one or more components of the cellular splicing machinery. A splicing modulator can also encompass or an agent that inhibits or blocks binding of one or more components of the cellular splicing machinery (e.g., to the target RNA sequence or an RNA comprising the target RNA sequence). In some embodiments, a splicing factor comprises a naturally occurring component of the cellular splicing machinery or a functional fragment or variant thereof. In some embodiments, a splicing factor comprises a recombinant and/or synthetic component of the cellular splicing machinery or a functional fragment or variant thereof. In some embodiments, a splicing modulator (e.g., a splicing factor) comprises Sam68, hnRNP G, SRSF1, hnRNP A1/A2, TDP-43, SRp-30c, PSF, or hnRNP M.

In some embodiments, the RNA effector comprises an RNA editing domain, e.g., as described below.

### RNA-Editing Domain

Certain polypeptides described herein include an RNA editing domain.

In some embodiments, an RNA editing domain produces a substitution in an RNA.

In some embodiments, an RNA editing domain produces an insertion or deletion in an RNA. In some embodiments, the RNA editing domain produces an insertion of less than 5, 4, 3, 2, or 1 nucleotides in the RNA. In some embodiments, the RNA editing domain produces a deletion of less than 5, 4, 3, 2, or 1 nucleotides in the RNA. In some embodiments, the RNA editing domain: (a) breaks a phosphodiester bond, producing a first portion of the RNA and a second portion of the RNA, (b) optionally adds or removes nucleotides from the first or second portion, and (c) rejoins the first portion with the second portion. In some embodiments, this RNA editing results in an insertion, deletion, or replacement of one or more nucleotides in the RNA. RNA editing to produce insertions and deletions is described, e.g., in Benne “RNA editing in trypanosomes” European Journal of Biochemistry 221:1 (1994) pages 9-23, which is herein incorporated by reference in its entirety.

In some embodiments, the RNA editing domain comprises the catalytic domain of an enzyme that edits one or more bases of a target RNA sequence, a functional fragment or variant thereof (e.g., a functional fragment or variant of a cytidine or adenosine deaminase). The RNA editing domain may be a polypeptide sequence comprising a catalytic domain of an RNA deaminase, e.g., an adenosine deaminase, a cytidine deaminase. For example, the RNA editing domain is the catalytic domain of an Adenosine Deaminase Acting on RNA (ADAR) (e.g., human ADAR 1, human ADAR2, human ADAR3, or human ADAR4); an Adenosine Deaminase Acting on tRNAs (ADAT), a Cytosine Deaminase Acting on RNA (CDAR). In embodiments, the catalytic domain of the deaminase comprises a sequence at least 80% identical (e.g., at least 85%, 87%, 90%, 92%, 95%, 98%, 99%, 100% identical) to a sequence having a GenBank Accession # identified in Table B. In embodiments, the catalytic domain of the deaminase comprises a sequence at least 80% identical (e.g., at least 85%, 87%, 90%, 92%, 95%, 98%, 99%, 100% identical) to a catalytic core domain sequence shown in Table B.

TABLE B
Catalytic core domain of cytidine and adenosine deaminases (Maas and Rich,
BioEssays 22: 790-802 (2000) John Wiley & Sons, Inc.)

| | | GenBank | Sequence alignment with clustal W1.8 |
|---|---|---|---|
| Gene | Species | Accession # | (catalytic core domain) |
| APOBEC1 | mouse | U22262 | SNHVEVNFLEKFTTERY-FRPTWFLSWSPCGECSR |
| APOBEC1 | human | L26234 | TNHVEVNFIKKFTSERD-FHPTWFLSWSPCWECSQ |
| APOBEC1 | rabbit | OCU10695 | TNHVEVNFLEKLTSEGR-LGPTWFLSWSPCWECSM |
| APOBEC2 | human | AF161698 | AAHAEEAFFNTILPAFD---PTWYVSSSPCAACAD |
| AID | mouse | AF132979 | GCHVELLFLRYISD-WD-LDPTWFTSWSPCYDCAR |
| ADAR1 | human | H5U10439 | DCHAEIISRRGFIRFLY-SELHLYISTAPCGDGAL |
| ADAR1 | X. laevis | XLU88065 | DCHAEVVSRRGFIRFLY-SQLHLYISTAPCGDGAL |
| ADAR2 | human | H5U76420 | DCHAEIISRRSLLRFLY-TQLHLYISTSPCGDARI |
| ADAR2 | rat | RN U43534 | DCHAEIISRRSLLRFLY-AQLHLYISTSPCGDARI |
| RED2 | rat | RN U74586 | DCHAEIVARRAFLHFLY-TQLHLYVSTSPCGDARL |
| ADAR | C. elegans | AF051275 | DCHAEILARRGLLRFLY-SEVHLFINTAPCGVARI |
| ADAR | D. | DMBN35H14a | DSHAEIVSRRCLLKYLY-AQFHLYINTAPCGDARI |
| | melanogaster | | |
| dCMP/CMP | Human | L12136 | VCHAELNAIMN-KNSTDVKGCSMYVALFPCNECAK |
| dCMP/CMP | Yeast | YSCDCD1 | CLHAEENALLEAGRDRVGQNATLYCDTCPCLTCSV |
| CDA | Human | L27943 | GICAERTAIQKAVSEGY-KDFMQDDFISPCGACRQ |
| CDA | E. coli | ECCDD | TVHAEQSAISHAWLSGE-KALAITVNYTPCGHCRQ |
| hypCDA | C. elegans | P30648 | VVHAEMNAIIN-KRCTTLHDCTVYVTLFPCNKCAQ |
| hypCDA | E. coli | P30134 | TAHAEIMALRQGGLVMQ-NYRTLYVTLEPCVMCAG |
| hypCDA | H. influenza | P44931 | TAHAEIIALRNGAKNIQ-NYRTLYVTLEPCTMCAG |
| ADAT1 | Human | AF125188 | DSHAEVIARRSFQRYLL-HQLVFFSSHTPCGDASI |
| ADAT1 | Yeast | SCE7297 | DCHAEILALRGANTVLL-NRIALYISRLPCGDASM |
| ADAT1 | D. | AF192530 | DSHAEVLARRGFLRFLY-QELHFLSTQTPCGDACI |
| | melanogaster | | |
| ADAT2 | Human | AL031320.6a | TRHAEMVAIDQVLDWCRQSGTVLYVTVEPCIMCAA |
| ADAT2 | Yeast | SCE242667 | VAHAEFMGIDQIKAMLG-SRGTLYVTVEPCIMCAS |
| ADAT3 | Human | AC012615.1a | LLHAVMVCVDLVARGQGRGGYDLYVTREPCAMCAM |
| ADAT3 | Yeast | SCE242668 | IDHSVMVGIRAVGERLR-EGVDVYLTHEPCSMCSM |

In some embodiments, an RNA editing domain comprises a deaminase that targets single stranded RNA (ssRNA). In some embodiments, an RNA editing domain comprises a deaminase that targets double stranded RNA (dsRNA). Without wishing to be bound by theory, although mRNA is typically a single stranded RNA, mRNA may comprise secondary structural elements that form dsRNA which may be edited by a deaminase that targets dsRNA. In some embodiments, compositions described herein may further comprise a nucleic acid with complementarity to a target RNA sequence (e.g., an antisense oligonucleotide) and which is capable of hybridizing to a target RNA sequence. Without wishing to be bound by theory, the dsRNA formed by a nucleic acid with complementarity to a target RNA sequence, e.g., an antisense oligonucleotide, and the target RNA sequence may allow the target RNA sequence to be targeted by a deaminase that targets dsRNA, e.g., in the absence of mRNA secondary structure that forms dsRNA. In some embodiments, a nucleic acid with complementarity to a target RNA sequence comprises DNA. In some embodiments, a nucleic acid with complementarity to a target RNA sequence comprises RNA. In some embodiments, a nucleic acid with complementarity to a target RNA sequence comprises one or more modified or synthetic nucleotides.

Exemplary nucleic acids with complementarity to a target RNA sequence (e.g., an antisense oligonucleotide), e.g., GluA2 mRNA or SMN2 mRNA, include but are not limited to SEQ ID NOs: 26-29.

70 nt targeting sequence for GLUA2
mRNA sequence (residue to be modified in bold/underline):
(SEQ ID NO: 26)
5′-ggcuauggcaucgcaacaccuaaaggauccucauuaAgguggguggaauaguauaacaauaugcuaaaug-3′
66 nt targeting sequence for SMN2 (PUF targeting in bold/underline):
(SEQ ID NO: 27)
5′-
UUUUUUAACUUCCUUUAUUUUCCUUACAGGGUUUUAGACAAAAUCAAAAAGAAGGAAGGUGCUCA
C-3′
Anti-sense oligonucleotide targeting sequence for GLUA2
(SEQ ID NO: 28)
5′-ACTATTCCACCCACCGTAATGAGGATCCTT-3′
Anti-sense oligonucleotide targeting sequence for SMN2
(SEQ ID NO: 29)
5′ - TCACTTTCATAATGCTGG - 3′

Exemplary RNA-editing domains include but are not limited to the RNA-editing domains of SEQ ID NOs: 14-21, or as encoded by SEQ ID NOs: 5-12. In some embodiments, an RNA-editing domain comprises an amino acid sequence with at least 80, 85, 90, 91, 92, 93, 94, 95, 96, 97, 98, or 99% identity to the RNA-editing domain of SEQ ID NOs: 14-21 (or comprising no more than 1, 2, 3, 4, 5, 6, 7, 8, 9, or 10 alterations relative thereto), or are encoded by a nucleic acid sequence with at least 80, 85, 90, 91, 92, 93, 94, 95, 96, 97, 98, or 99% identity to the RNA-editing domain encoding sequence of SEQ ID NOs: 5-12.

### Linkers

In some embodiments, polypeptides described herein may include one or more linkers. In some embodiments, RNA base-binding motifs in an RNA-binding domain are joined by a linker. In some embodiments, the RNBA-binding domain and RNA-editing domain have a linker between them. A linker may be a chemical bond, e.g., one or more covalent bonds or non-covalent bonds. In some embodiments links are covalent. In some embodiments, links are non-covalent. In some embodiments, a linker is a peptide linker. Such a linker may be between 2-30 amino acids, or longer. In some embodiments, a linker is used, e.g., to provide molecular flexibility of secondary and tertiary structures, or to allow separate domains or motifs to function (e.g., to bind a target) while minimizing steric hindrance. A linker may comprise flexible, rigid, and/or cleavable linkers described herein. In some embodiments, a linker includes at least one glycine, alanine, and serine amino acids to provide for flexibility. In some embodiments, a linker is a hydrophobic linker, such as including a negatively charged sulfonate group, polyethylene glycol (PEG) group, or pyrophosphate diester group. In some embodiments, a linker is cleavable to selectively release a moiety (e.g. a domain) from another, but sufficiently stable to prevent premature cleavage.

Commonly used flexible linkers have sequences consisting primarily of stretches of Gly and Ser residues (“GS” linker). Flexible linkers may be useful for joining domains that require a certain degree of movement or interaction and may include small, non-polar (e.g. Gly) or polar (e.g. Ser or Thr) amino acids. Incorporation of Ser or Thr can also maintain the stability of a linker in aqueous solutions by forming hydrogen bonds with water molecules, and therefore reduce unfavorable interactions between a linker and protein moieties.

Rigid linkers are useful to keep a fixed distance between domains and to maintain their independent functions. Rigid linkers may also be useful when a spatial separation of domains is critical to preserve the stability or bioactivity of one or more components in the fusion. Rigid linkers may have an alpha helix-structure or Pro-rich sequence, (XP)n, with X designating any amino acid, preferably Ala, Lys, or Glu.

Cleavable linkers may release free functional domains in vivo. In some embodiments, linkers may be cleaved under specific conditions, such as presence of reducing reagents or proteases. In vivo cleavable linkers may utilize reversible nature of a disulfide bond. One example includes a thrombin-sensitive sequence (e.g., PRS) between the two Cys residues. In vitro thrombin treatment of CPRSC results in the cleavage of a thrombin-sensitive sequence, while a reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under certain conditions, in specific cells or tissues, or constrained within certain cellular compartments. Specificity of many proteases offers slower cleavage of the linker in constrained compartments.

Examples of linking molecules include a hydrophobic linker, such as a negatively charged sulfonate group; lipids, such as a poly (—CH2-) hydrocarbon chains, such as polyethylene glycol (PEG) group, unsaturated variants thereof, hydroxylated variants thereof, amidated or otherwise N-containing variants thereof, noncarbon linkers; carbohydrate linkers; phosphodiester linkers, or other molecule capable of covalently linking two or more components of a disrupting agent (e.g. two polypeptides). Non-covalent linkers are also included, such as hydrophobic lipid globules to which the polypeptide is linked, for example through a hydrophobic region of a polypeptide or a hydrophobic extension of a polypeptide, such as a series of residues rich in leucine, isoleucine, valine, or perhaps also alanine, phenylalanine, or even tyrosine, methionine, glycine or other hydrophobic residue. Components of a disrupting agent may be linked using charge-based chemistry, such that a positively charged component of a disrupting agent is linked to a negative charge of another component or nucleic acid.

### Methods of Making Compositions

Methods of making recombinant proteins or polypeptides (e.g., polypeptides described herein) are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

A protein or polypeptide of compositions of the present disclosure can be biochemically synthesized, e.g., by employing standard solid phase techniques. Such methods include exclusive solid phase synthesis, partial solid phase synthesis methods, fragment condensation, classical solution synthesis. These methods can be used when a peptide is relatively short (i.e., 10 kDa) and/or when it cannot be produced by recombinant techniques (e.g., not encoded by a nucleic acid sequence) and therefore involves different chemistry. Solid phase synthesis procedures are well known in the art and further described by John Morrow Stewart and Janis Dillaha Young, Solid Phase Peptide Syntheses, 2nd Ed., Pierce Chemical Company, 1984; and Coin, I., et al., Nature Protocols, 2:3247-3256, 2007.

For longer polypeptides, recombinant methods may be used. Methods of making a recombinant therapeutic polypeptide are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013). Exemplary methods for producing a therapeutic pharmaceutical protein or polypeptide involve expression in mammalian cells, although recombinant proteins can also be produced using insect cells, yeast, bacteria, or other cells under control of appropriate promoters. Mammalian expression vectors may comprise nontranscribed elements such as an origin of replication, a suitable promoter, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

In cases where large amounts of the polypeptide are desired, it can be generated using techniques such as described by Brian Bray, Nature Reviews Drug Discovery, 2:587-593, 2003; and Weissbach & Weissbach, 1988, Methods for Plant Molecular Biology, Academic Press, NY, Section VIII, pp 421-463. Various mammalian cell culture systems can be employed to express and manufacture recombinant protein. Examples of mammalian expression systems include CHO cells, COS cells, HeLA and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein. Viral and bacteriophage expression vectors are generated by traditional genetic techniques. For gene transfer to dividing and non-dividing cells, viral expression vectors may include Lentivirus or Adenovirus (AAV). For gene transfer to the central nervous system (CNS), either AAV vectors or M13 bacteriophage vectors may be used.

Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010).

Nucleic acids as described herein or nucleic acids encoding a protein described herein, may be incorporated into a vector. Vectors, including those derived from retroviruses such as lentivirus, are suitable tools to achieve long-term gene transfer since they allow long-term, stable integration of a transgene and its propagation in daughter cells. Examples of vectors include expression vectors, replication vectors, probe generation vectors, and sequencing vectors. An expression vector may be provided to a cell in the form of a viral vector. Viral vector technology is well known in the art, and described in a variety of virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

Expression of natural or synthetic nucleic acids is typically achieved by operably linking a nucleic acid encoding the gene of interest to a promoter, and incorporating the construct into an expression vector. Vectors can be suitable for replication and integration in eukaryotes. Typical cloning vectors contain transcription and translation terminators, initiation sequences, and promoters useful for expression of the desired nucleic acid sequence.

Additional promoter elements, e.g., enhancing sequences, may regulate frequency of transcriptional initiation. Typically, these sequences are located in a region 30-110 bp upstream of a transcription start site, although a number of promoters have recently been shown to contain functional elements downstream of transcription start sites as well. Spacing between promoter elements frequently is flexible, so that promoter function is preserved when elements are inverted or moved relative to one another. In a thymidine kinase (tk) promoter, spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. In some embodiments of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, an actin promoter, a myosin promoter, a hemoglobin promoter, and a creatine kinase promoter.

The present disclosure should not interpreted to be limited to use of any particular promoter or category of promoters (e.g. constitutive promoters). For example, in some embodiments, inducible promoters are contemplated as part of the present disclosure. In some embodiments, use of an inducible promoter provides a molecular switch capable of turning on expression of a polynucleotide sequence to which it is operatively linked, when such expression is desired. In some embodiments, use of an inducible promoter provides a molecular switch capable of turning off expression when expression is not desired. Examples of inducible promoters include, but are not limited to a metallothionine promoter, a glucocorticoid promoter, a progesterone promoter, and a tetracycline promoter.

In some embodiments, an expression vector to be introduced can also contain either a selectable marker gene or a reporter gene or both to facilitate identification and selection of expressing cells from the population of cells sought to be transfected or infected through viral vectors. In some aspects, a selectable marker may be carried on a separate piece of DNA and used in a co-transfection procedure. Both selectable markers and reporter genes may be flanked with appropriate expression control sequences to enable expression in the host cells. Useful selectable markers may include, for example, antibiotic-resistance genes, such as neo, etc.

In some embodiments, reporter genes may be used for identifying potentially transfected cells and/or for evaluating the functionality of expression control sequences. In general, a reporter gene is a gene that is not present in or expressed by a recipient source (of a reporter gene) and that encodes a polypeptide whose expression is manifested by some easily detectable property, e.g., enzymatic activity or visualizable fluorescence. Expression of a reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, a construct with a minimal 5′ flanking region that shows highest level of expression of reporter gene is identified as a promoter. Such promoter regions may be linked to a reporter gene and used to evaluate agents for ability to modulate promoter-driven transcription.

### Applications

The RNA-editing compositions (e.g., polypeptides, nucleic acids, vectors and host cell described herein) can address therapeutic needs, for example, by correcting a loss-of-function mutation (e.g., one or more point mutation) in an RNA in a cell, tissue or subject. For example, the RNA-editing compositions (e.g., polypeptides, nucleic acids, vectors and host cell described herein) may be used to treat diseases associated with a mutation, e.g., one or more point mutation, in a gene.

The compositions described herein (e.g., polypeptides, nucleic acids, vectors and host cell described herein) may be used to treat a disease or condition. In some embodiments, the disease is selected from Meier-Gorlin syndrome, Seckel syndrome 4, Joubert syndrome 5, Leber congenital amaurosis 10; Charcot-Marie-Tooth disease, type 2; Charcot-Marie-Tooth disease, type 2; Usher syndrome, type 2C; Spinocerebellar ataxia 28; Spinocerebellar ataxia 28; Spinocerebellar ataxia 28; Long QT syndrome 2; Sjogren-Larsson syndrome; Hereditary fructosuria; Hereditary fructosuria; Neuroblastoma; Neuroblastoma; Kallmann syndrome 1; Kallmann syndrome 1; Kallmann syndrome 1; Metachromatic leukodystrophy, Rett syndrome, Amyotrophic lateral sclerosis type 10, Li-Fraumeni syndrome, Cystic fibrosis, Hurler Syndrome, alpha-1-antitrypsin (A1AT) deficiency, Parkinson's disease, Alzheimer's disease, albinism, Amyotrophic lateral sclerosis, Asthma, b-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (I BD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, Sturge-Weber Syndrome, and cancer. In some embodiments, the disclosure is directed to the use of a composition described herein (e.g., a polypeptide, nucleic acid, vector, or host cell described herein) in the manufacture of a medicament for the treatment or prevention of a disease or disorder (e.g., a genetic disorder) selected from a disease or disorder listed herein.

### Formulation, Administration and Delivery

In various embodiments, the disclosure provides pharmaceutical compositions of polypeptides, nucleic acids, vectors and host cells described herein, formulated with a pharmaceutically acceptable excipient. Pharmaceutically acceptable excipient includes an excipient that is useful in preparing a pharmaceutical composition that is generally safe, nontoxic, and desirable, and includes excipients that are acceptable for veterinary use as well as for human pharmaceutical use. Such excipients may be aqueous or non-aqueous. Appropriate excipients may aid in, e.g., stability, solubility, buffering, of the composition. Formulation of protein therapeutics is described in Meyer (Ed.), Therapeutic Protein Drug Products: Practical Approaches to formulation in the Laboratory, Manufacturing, and the Clinic, Woodhead Publishing Series (2012).

Pharmaceutical compositions according to the present disclosure may be delivered in a therapeutically effective amount. A precise therapeutically effective amount is an amount of a composition, e.g., polypeptides, nucleic acids, vectors and host cells described herein, that has a desired therapeutic effect on the subject. This amount will vary depending upon a variety of factors, including but not limited to characteristics of a therapeutic compound (including activity, pharmacokinetics, pharmacodynamics, and bioavailability), physiological condition of a subject (including age, sex, disease type and stage, general physical condition, responsiveness to a given dosage, and type of medication), nature of a pharmaceutically acceptable carrier or carriers in a formulation, and/or route of administration. Modes of administration to a subject may include systemic, parenteral, enteral or local.

In some embodiments a polypeptide or nucleic acid composition described herein may be delivered to a cell, tissue or subject using a vector. The vector may be, e.g., a plasmid or a virus. In some embodiments delivery is in vivo, in vitro, ex vivo, or in situ. In some embodiments the virus is an adeno associated virus (AAV), a lentivirus, an adenovirus. In some embodiments a polypeptide or nucleic acid composition described herein is delivered to cells with a viral-like particle or a virosome. In some embodiments the delivery uses more than one virus, viral-like particle or virosome.

Liposomal Formulations

Exemplary formulations suitable as vehicles or carriers for delivery of a polypeptide, pharmaceutical composition, nucleic acid, vector, composition, or host cell described herein, include microemulsions, monolayers, micelles, bilayers, vesicles or lipid particles. These formulations provide a biocompatible and biodegradable delivery system for a polypeptide, pharmaceutical composition, nucleic acid, vector, composition, or host cell described herein.

Liposomes provide an example of lipid particles, which are composed of amphiphilic lipids arranged in a spherical bilayer or bilayers. Liposomes are unilamellar or multilamellar vesicles which have a membrane formed from a lipophilic material and an aqueous interior. The aqueous portion comprises the a polypeptide, pharmaceutical composition, nucleic acid, vector, composition, or host cell described herein, to be delivered. Cationic liposomes possess the advantage of being able to fuse to the cell wall. Non-cationic liposomes, although not able to fuse as efficiently with the cell wall, are taken up by macrophages in vivo.

Liposomes have several advantages including a small diameter; biocompatibility and biodegradability; ability to incorporate a wide range of contents, e.g., water and lipid soluble drugs. Liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Exemplary non-ionic liposomal systems suitable for delivery of drugs to the skin include systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P. Pharma. Sci., 1994, 4, 6, 466).

Liposomes can be sterically stabilized to include one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M}1, or (B) is derivatized with one or more hydrophilic polymers, such as a polyethylene glycol (PEG) moiety (Allen et al., FEBS Letters, 1987, 223, 42; Wu et al., Cancer Research, 1993, 53, 3765). Long-circulating, e.g., stealth, liposomes can also be employed. Such liposomes are generally described in U.S. Pat. No. 5,013,556. The compounds disclosed herein can also be administered by controlled release means and/or delivery devices such as those described in U.S. Pat. Nos. 3,845,770; 3,916,899; 3,536,809; 3,598,123; and 4,008,719.

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

Liposomes comprising lipids can be derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C_{1215G}, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

A number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes. U.S. Pat. No. 5,665,710 to Rahman et al. describes certain methods of encapsulating oligodeoxynucleotides in liposomes.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations. The use of surfactants in drug products, formulations and in emulsions has been reviewed (Rieger, in Pharmaceutical Dosage Forms, Marcel Dekker, Inc., New York, N.Y., 1988, p. 285).

Another example of delivery vehicles include nanostructured lipid carriers (NLCs), which are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. For a review, see, e.g., Li et al. 2017, Nanomaterials 7, 122; doi:10.3390/nano7060122.

In some embodiments, a nucleic acid, vector, or composition described herein can be encapsulated in a lipid formulation, e.g., to form a nucleic acid-lipid particle. Nucleic acid lipid particles typically contain a cationic lipid, a non-cationic lipid, and a lipid that prevents aggregation of the particle (e.g., a PEG-lipid conjugate). These particles are useful for systemic applications, as they exhibit extended circulation lifetimes following intravenous (i.v.) injection and accumulate at distal sites (e.g., sites physically separated from the administration site). Particles which include an encapsulated condensing agent-nucleic acid complex as set forth in PCT Publication No. WO 00/03683. The particles typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO 96/40964.

In one embodiment, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1.

The cationic lipid may be, for example, N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (Tech G1), or a mixture thereof. The cationic lipid may comprise from about 20 mol % to about 50 mol % or about 40 mol % of the total lipid present in the particle.

In one embodiment, the lipid particle includes 40% 2, 2-Dilinoleyl-4-dimethylaminoethyl-[1,3]-dioxolane: 10% DSPC: 40% Cholesterol: 10% PEG-C-DOMG (mole percent) with a particle size of 63.0±20 nm and a 0.027 siRNA/Lipid Ratio.

The non-cationic lipid may be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid may be from about 5 mol % to about 90 mol %, about 10 mol %, or about 58 mol % if cholesterol is included, of the total lipid present in the particle.

The conjugated lipid that inhibits aggregation of particles may be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C]₈). The conjugated lipid that prevents aggregation of particles may be from 0 mol % to about 20 mol % or about 2 mol % of the total lipid present in the particle.

In some embodiments, the nucleic acid-lipid particle further includes cholesterol at, e.g., about 10 mol % to about 60 mol % or about 48 mol % of the total lipid present in the particle.

In one embodiment, the formulations is an MC3 comprising formulations are described, e.g., in International Application No. PCT/US10/28224, filed Jun. 10, 2010, which is hereby incorporated by reference. The synthesis and structure of MC3 containing formulations is described in, e.g., pages 114-119 of WO 2013/155204, incorporated by reference. In some embodiments, the MC3 formulation comprises a preparation of DLin-M-C3-DMA (i.e., (6Z,9Z,28Z,31Z)-heptatriaconta-6,9,28,31-tetraen-19-yl 4-(dimethylamino)butanoate)

In some embodiment, a polypeptide, nucleic acid, vector or host cell composition described herein may be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles (LNPs) are another example of a carrier that provides a biocompatible and biodegradable delivery system for the pharmaceutical compositions described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for the compositions and systems described herein. For a review, see Ha et al. July 2016. Acta Pharmaceutica Sinica B. Volume 6, Issue 4, Pages 287-296; https://doi.org/10.1016/j.apsb.2016.02.001.

All publications, patent applications, patents, and other publications and references (e.g., sequence database reference numbers) cited herein are incorporated by reference in their entirety. For example, all GenBank, Unigene, and Entrez sequences referred to herein, e.g., in any Table or Example herein, are incorporated by reference. Unless otherwise specified, the sequence accession numbers specified herein, including in any Table herein, refer to the database entries current as of Nov. 29, 2018. When one gene or protein references a plurality of sequence accession numbers, all of the sequence variants are encompassed.

### EXAMPLES

The invention is further illustrated by the following examples. The examples are provided for illustrative purposes only and are not to be construed as limiting the scope or content of the invention in any way.

### Example 1: Design and Expression of Fusion Construct

This example describes the design and production of fusion proteins comprising an RNA-binding domain fused to an RNA editing domain.

RNA binding domain: an 8 nucleotide sequence of a target RNA is converted to a topological protein recognition code as described above and by Cheong and Tanaka. 2006. PNAS vol. 103, 37: 13635-9. This code is incorporated into the RNA binding domain of PUM1 (SEQ ID NO:1) which is Gly 828 to Gly 1176 of the amino acid sequence of GenBank: AAG31807.1, using, e.g., site directed mutagenesis of a pTYB3 plasmid encoding PUM1 with the Quick Change II XL Site Directed Mutagenesis Kit (Stratagene, La Jolla, Calif.).

RNA editing domain: a construct is designed containing the catalytically active domain of human ADAR2 (hADAR2DD) (aa 276-701 of SEQ ID NO:2) with the E488Q mutation for enhanced deaminase activity as described in Kuttan and Bass. 2012. PNAS 2012 and Phelps, Kelly J et al Nucleic Acids Research 2015.

The corresponding sequences of the ORFs of the RNA-binding and RNA-editing domains described above are synthesized from the aforementioned plasmids and amplified with polymerase chain reaction (PCR) using the primers described in Sinnamon et al. 2017. PNAS 114.44 (2017): E9395-E9402, then cloned into an ampicillin resistant pcDNA-CMV vector backbone using the Gibson Assembly® protocol (New England Biolabs), per the manufacturer's instructions, with the RNA-binding domain being fused in frame at the C-terminus of hADAR2DD. Constructs are confirmed with DNA sequencing.

The fusion protein can be expressed in E. coli strain BL21 (DE3) cells. Plasmids are expressed in E. coli cells are grown in Lennox LB media (Sigma, USA) at 37° C. overnight. Cells are harvested by centrifugation at 6000 g for 30 min, then resuspended in a lysis buffer, sonicated, and purified as described in Wang, X. et al 2002. The lysates are cleared by spinning at 20,000 rpm for 30 min, then loaded onto a 10 ml Ni-NTA agarose column (Qiagen, USA). The elute is purified with a Sephedex75 gel filtration column then concentrated to ˜5.5 mg/ml in 10 mM Tris (pH 7.4), 150 mM NaCl, and 2 mM dithiothreitol (DTT). The aliquots are flash-frozen in liquid nitrogen and stored at −80° C. as described in Wang, X. et al. 2002 and Dawson, T. R. 2003.

Protein purification is confirmed with SDS/PAGE and Coomassie blue staining. The peak fraction of fusion protein is serially diluted in 100 μg/ml bovine serum albumin (BSA) and resolved by SDS-PAGE comparing to known concentrations of BSA as a standard.

### Example 2: Editing Efficiency Assay

This example describes an assay to evaluate the editing efficiency of a fusion protein prepared as described herein.

Panoply™ Human ADAR knockdown HEK293 cells (Creative Biogene, Shirley, N.Y.) are seeded at a density of (3×105/well) onto poly-d-lysine-coated 24-well plates maintained in Dulbecco's Modified Eagle's Medium (DMEM) supplemented with 10% fetal bovine serum, 1% penicillin-streptomycin solution, 1 mM sodium pyruvate, and 2 mM glutamine at 37 C, 5% CO2 for 24 hours. Cells are transfected with constructs of the fusion protein with lipofectamine 2000 per the manufacturers protocol and maintained for 72 h after transfection. RNA editing efficiency is validated by isolating total RNA from cells with TRIZOL (Invitrogen) following the manufacturer's instructions, then DNaseI treatment on 1 ug of total RNA, followed by reverse transcription. cDNA is synthesized with iScript cDNA synthesis kit (BioRad, Hercules, Calif.) with randomly selected RT-primers and subjected to PCR-amplification. The products are directly sequenced to compare (A) to inosine (I) substitution of ADAR deficient cells transfected with the subject fusion protein to their time-matched controls as described in Wettengel et al. 2016. Nucleic Acids Research 45, 5: 2797-2808.

### Example 3: RNA Editing of an Exemplary ORF Point Mutation

This example demonstrates the ability of a fusion polypeptide of the invention to edit an ORF mRNA.

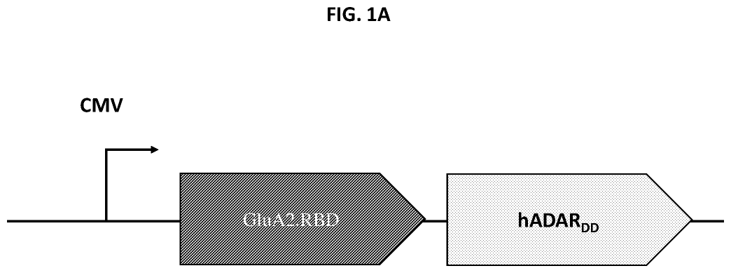

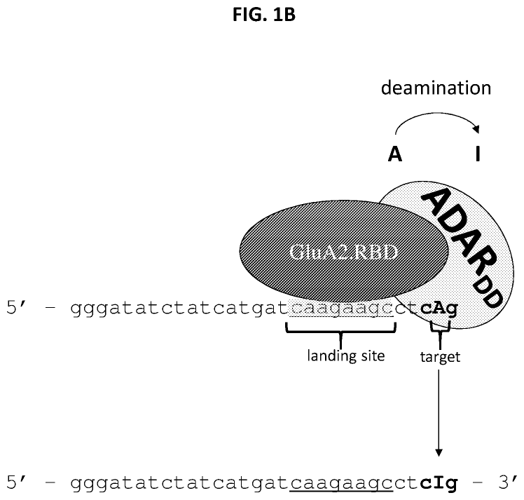

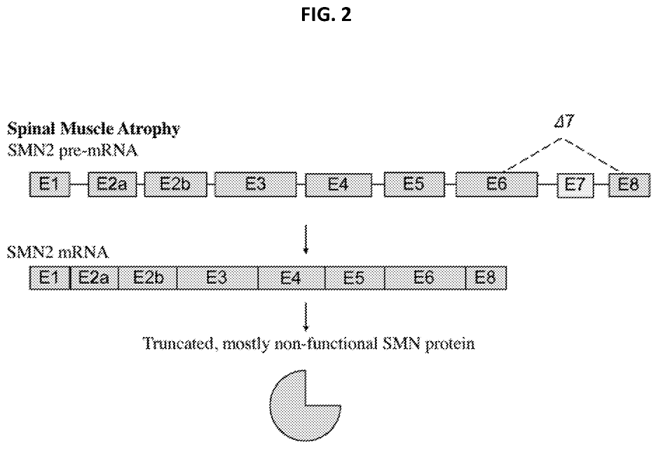

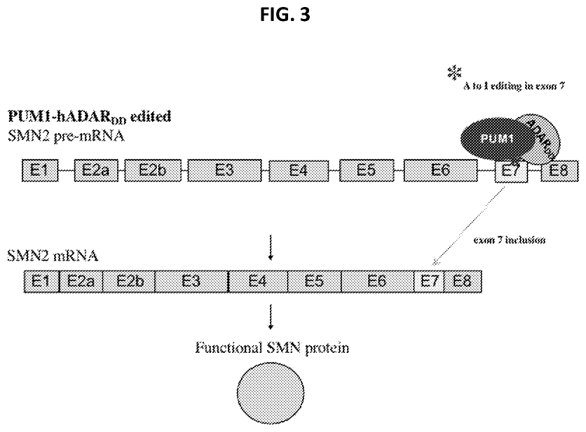

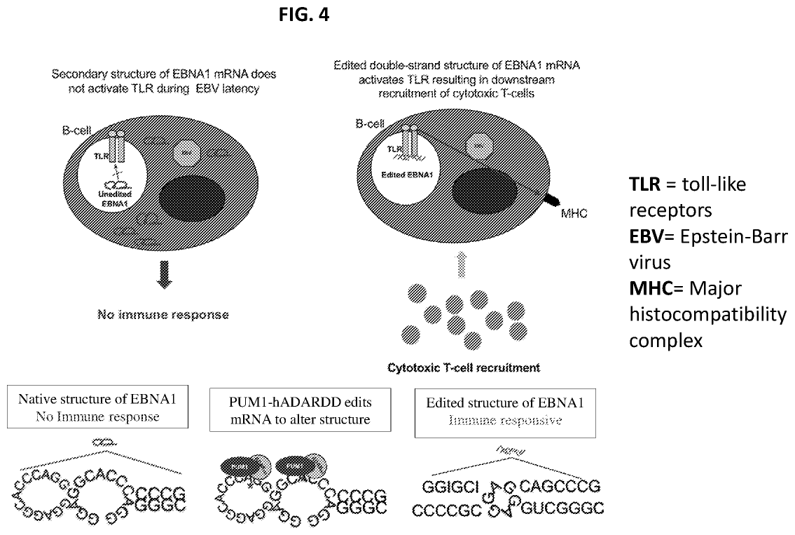

In this example, RNA editing is used to alter the sequence and function of a transport protein related to dysregulated ion flux in a neuronal disorder. In neurons, nearly 99% of the GluA2 subunit of the α-Amino-3-hydroxy-5-methyl-4-isoxazolepropionic acid (AMPA) complex is edited by the naturally occurring ADAR complex. This editing of the GluA2 converts a codon for a polar glutamine (Q) to a codon for a charged arginine (R). This conversion results in a loss of Ca2+ permeability in motor neurons where GluA2 has been edited. Patients with Amyotrophic Lateral Sclerosis (ALS) exhibit loss of this GluA2 editing and resulting calcium related excitotoxity in motor neurons. A polypeptide of the invention can be used to edit the target codon of human GluA2 mRNA to produce the corrective amino acid substitution Q607R in the resulting protein. The codon for amino acid 607 of GluA2 comprises nucleotides 1555-1557 of the human GluA2 nucleotide sequence (NCBI reference sequence NM_001083620.1). The effector polypeptide of the invention thus includes the catalytic domain of a human ADAR which will edit the relevant codon CAG (glutamine) to CIG, which is read as CGG (arginine), linked to an RNA-binding (targeting) domain that will specifically bind a sequence upstream of nucleotides 1555-1557 of the human GluA2 nucleotide sequence (See FIG. 1). In particular, the effector polypeptide is constructed as follows:

RNA-binding domain: The sequence of PUM1-HD is altered to bind an 8 nucleotide sequence upstream of the target codon to be edited (amino acids 1545-1552 of the human GluA2 nucleotide sequence: caagaagc), to create GluA2.RBD as follows:

Repeat 1: Mutation S863C and Q867R
Wild-type: HIMEFSQDQHGSRFIQLKLERATPAERQLVFNEILQ
Mutant for GluA2 recognition: HIMEFSQDQHGCRFIRLKLERATPAERQLVFNEILQ
Repeat 2: Mutation N899S and Y867R
Wild-type: AAYQLMVDVFGNYVIQKFFEFGSLEQKLALAERIRG
Mutant for GluA2 recognition: AAYQLMVDVFGSRVIQKFFEFGSLEQKLALAERIRG
Repeat 3: Mutation C935S, R936N and Q939E
Wild-type: HVLSLALQMYGCRVIQKALEFIPSDQQNEMVRELDG
Mutant for GluA2 recognition: AAYQLMVDVFGSNVIEKFFEFGSLEQKLALAERIRG
Repeat 4: Mutation N971S and H972R
Wild-type: HVLKCVKDQNGNHVVQKCIECVQPQSLQFIIDAFKG
Mutant for GluA2 recognition: HVLKCVKDQNGSRVVQKCIECVQPQSLQFIIDAFKG
Repeat 5: No mutation
Wild-type: QVFALSTHPYGCRVIQRILEHCLPDQTLPILEELHQ
Mutant for GluA2 recognition: QVFALSTHPYGCRVIQRILEHCLPDQTLPILEELHQ
Repeat 6: N1043S, Y1044N and Q1047E
Wild-type: HTEQLVQDQYGNYVIQHVLEHGRPEDKSKIVAEIRG
Mutant for GluA2 recognition: HTEQLVQDQYGSNVIEHVLEHGRPEDKSKIVAEIRG
Repeat 7: No mutation
Wild-type: NVLVLSQHKFASNVVEKCVTHASRTERAVLIDEVCTMNDGPHS
Mutant for GluA2 recognition:
NVLVLSQHKFASNVVEKCVTHASRTERAVLIDEVCTMNDGPHS
Repeat 8: N1122C and Q1126R
Wild-type: ALYTMMKDQYANYVVQKMIDVAEPGQRKIVMHKIRP
HIATLRKYTYGKHILAKLEKYYMKNGVDLG
Mutant for GluA2 recognition:
ALYTMMKDQYACYVVRKMIDVAEPGQRKIVMHKIRPHIATLRKYTYGKHILAKLEKYY
MKNGVDLG

RNA-editing domain: the RNA-editing domain comprises a catalytic domain of human ADAR2DD and is designed and made as in Example 1. PCR ligation and amplification of the fusion construct GluA2.RBD-ADAR2DD is performed as described in Adamala, et al. 2016. PNAS 113.19: E2579-E2588. Alternative exemplary constructs for use in the methods of this Example include RNA editing domains, RNA effectors, and/or polypeptides disclosed herein (and/or nucleic acids encoding the same), e.g., SEQ ID NOs: 16 or 17 (and/or SEQ ID NOs: 7 or 8). Alternative exemplary target RNA sequences include mRNA sequence corresponding to nucleotides 1537-1552 of human GluA2 (Reference sequence NM_000826) or a sequence within 50 nucleotides of nucleotides 1537-1552 of human GluA2.

Assay: The GluA2.RBD-ADAR2DD construct described above is used in the following test model:

Mouse neuroblastoma (N2A) cells, cells are seeded at a density of 1×10³ cells per well in 24-well plates and maintained in Eagle's Minimum Essential Medium (EMEM) supplemented with 10% fetal bovine serum, 1% penicillin-streptomycin solution, 1 mM sodium pyruvate, and 2 mM glutamine at 37 C, 5% CO₂ overnight. After 24 h, cells are transfected with ADAR siRNA lentivirus (abm cat: iV037759a) plasmids. ADAR knockdown is confirmed by western blot for ADAR expression levels.

After 24 hours, ADAR deficient N2A cells in Opti-MEM reduced serum media (Thermo Fisher Scientific) are transfected with Lipofectamine 2000 (Thermo Fisher Scientific) and 125 ng of the GluA2.RBD-ADAR2DD plasmid described above. Following 72 h, RNA editing is validated by isolating total RNA from cells with TRIZOL (Invitrogen) following protocol on manufacturer's website, then DNaseI treatment on 1 ug of total RNA, followed by reverse transcription. cDNA is synthesized with iScript cDNA synthesis kit (BioRad, Hercules, Calif.) with GluA2 RT-primers (fwd: CCATCGAAAGTGCTGAGGAT and rev: AGGGCTCTGCACTCCTCATA) and subjected to PCR-amplification. The products are directly sequenced to compare the rate of (A) to inosine (I) substitution of ADAR deficient cells transfected with GluA2.RBD-ADAR2DD to their time-matched controls with no ADAR activity as described in Wettengel, Jacqueline et al.

### Example 4: Editing of a Pre-mRNA to Generate Alternative Spliced Products

In Spinal Muscle Atrophy (SMA), the leading genetic cause of infant mortality, SMN protein is lacking due to a mutation or absence of the SMN1 gene. (Hua et al. 2007. PLoS biology vol. 5,4: e73.) Humans possess a SMN2 gene (Homo sapiens survival of motor neuron 2, centromeric (SMN2), RefSeqGene on chromosome 5 NCBI Reference Sequence: NG_008728.1) almost identical SMN1 capable of SMN protein production; however, a critical cytosine (C) to thymidine (T) mutation at the 6th position (C6U transition in transcript) of exon 7 and an adenosine (A) to guanosine (G) transition at the 100th position (A100G) of intron 7 reduces the recognition of splice sites resulting in the skipping of exon 7 in pre-mRNA splicing events. (Singh et al. 2012. PLoS One 7.11: e49595). Due to the skipped exon, the subsequent SMN protein is unstable and partially functional, leading to the SMA phenotype. This example describes the design and making of an exemplary composition described herein that could reduce the ‘splicing out’ of exon 7 in the pre-mRNA of SMN2 thereby rescuing SMN production and abrogating the disease phenotype.

### Plasmid Construction

RNA-binding domain: SMN2 pre-mRNA is modified to drive exon 7 inclusion with a an SMN2 RNA binding-hADARDD fusion construct. The target sequences of SMN2 that potentiate the inclusion of exon 7 are described in Hua et al. 2007. PLoS biology vol. 5, 4:e73. To target exon 7 of SMN2, we perform site directed mutagenesis of the PUM1-HD to target an 8-nucleotide sequence: UUAGACAA (pos. 27003-27010 of human SMN2 NCBI reference sequence NG_008728.1)

Mutations to be made to PUM 1 are as follows:

Repeat 1: Mutation S863N and R864Y
Wild-type: HIMEFSQDQHGSRFIQLKLERATPAERQLVFNEILQ
Mutant for SMN2 recognition: HIMEFSQDQHGNYFIQLKLERATPAERQLVFNEILQ
Repeat 2: No Mutation
Wild-type: AAYQLMVDVFGNYVIQKFFEFGSLEQKLALAERIRG
Mutant for SMN2 recognition: AAYQLMVDVFGNYVIQKFFEFGSLEQKLALAERIRG
Repeat 3: No Mutation
Wild-type: HVLSLALQMYGCRVIQKALEFIPSDQQNEMVRELDG
Mutant for SMN2 recognition: AAYQLMVDVFGCRVIQKFFEFGSLEQKLALAERIRG
Repeat 4: N971S, H972N and Q975E
Wild-type: HVLKCVKDQNGNHVVQKCIECVQPQSLQFIIDAFKG
Mutant for SMN2 recognition: HVLKCVKDQNGSNVVEKCIECVQPQSLQFIIDAFKG
Repeat 5: No mutation
Wild-type: QVFALSTHPYGCRVIQRILEHCLPDQTLPILEELHQ
Mutant for SMN2 recognition: QVFALSTHPYGCRVIQRILEHCLPDQTLPILEELHQ
Repeat 6: N1043C and Q1047R
Wild-type: HTEQLVQDQYGNYVIQHVLEHGRPEDKSKIVAEIRG
Mutant for SMN2 recognition: HTEQLVQDQYGCYVIRHVLEHGRPEDKSKIVAEIRG
Repeat 7: S1079C, N108OR and E1083Q
Wild-type: NVLVLSQHKFASNVVEKCVTHASRTERAVLIDEVCTMNDGPHS
Mutant for SMN2 recognition:
NVLVLSQHKFACRVVQKCVTHASRTERAVLIDEVCTMNDGPHS
Repeat 8: N1122C and Y1123R
Wild-type: ALYTMMKDQYANYVVQKMIDVAEPGQRKIVMHKIRP
HIATLRKYTYGKHILAKLEKYYMKNGVDLG
Mutant for SMN2 recognition: ALYTMMKDQYACRVVQKMIDVAEPGQRKIVMHKIRP
HIATLRKYTYGKHILAKLEKYYMKNGVDLG

RNA-editing domain: the RNA-editing domain comprises a catalytic domain of human ADAR2DD and is designed and made as in Example 1.

PCR ligation and amplification of the fusion construct SMN2.RBD-ADAR2DD is performed as described in Adamala, et al. 2016. PNAS 113.19: E2579-E2588. Alternative exemplary constructs for use in the methods of this Example include RNA editing domains, RNA effectors, and/or polypeptides disclosed herein (and/or nucleic acids encoding the same), e.g., SEQ ID NOs: 18 or 19 (and/or SEQ ID NOs: 9 or 10). Alternative exemplary target RNA sequences include mRNA sequence corresponding to nucleotides 31,995-32,010 of human SMN2 (Reference sequence NM-022876) or a sequence within 50 nucleotides of nucleotides 31, 995-32,010 of human SMN2.

### Cell Culture and Transfection

Human SMA type I fibroblast (Coriell Repositories) cells are plated 24 hours prior to transfection and maintained in DMEM supplemented with 10% of non-inactivated FBS, 37° C., 5% CO2. At ˜50% confluence, cells are transiently transfected with 0.5 μg SMN2.RBD-hADARDD plasmid. 4 h later, media is replaced with fresh medium. Total RNA is extracted after 48 h transfection.

### RT-PCR for Exon Inclusion

RT-PCR analysis for detection of exon 7 splicing of SMN2 is performed on the test cells described above as previously described in Cho et al. 2014. Biochimica et Biophysica Acta (BBA)-Gene Regulatory Mechanisms 1839.6: 517-525. Control cells are transfected with a plasmid expressing hADARDD without an RNA-binding fusion.

Total RNA is extracted from the control and test mammalian cells by RiboEx reagent (Geneall) and ethanol precipitation. Reverse transcription is performed in a total volume of 20 μl, containing 1 μg RNA, 0.5 μg oligo-dT, dNTP mix (0.5 mM each dNTP), 6 mM MgCl2, 4 μl of 5× ImProm-II™ reaction buffer and 1 μl of ImProm-II™ reverse transcriptase (Promega). RT-PCR amplification of SMN+exon 7, SMN−exon 7 and GAPDH control is conducted and PCR products (amplified using, e.g., the exon 6 and exon 8 PCR primers of Cho et al.) are analyzed on 2% agarose gels with ethidium bromide solution (0.5μ/ml). Test cells produce a larger SMN2 mRNA which includes exon 7. PCR products are digested with DdeI (NEB) and loaded onto 5% native polyacrylamide gels for detection.

### Example 5: Editing the Sequence of EBNA1 to Induce Anti-Viral Response to Epstein-Barr Virus

Epstein-Barr Virus (EBV) causes mononucleosis and is associated with many human cancers including Burkitt lymphoma, Hodgkin's, and nasopharyngeal carcinomas (Tellam et al. 2008. PNAS 105.27: 9319-9324). Following initial lytic infection, EBV has been shown to avoid immune surveillance and persist in a latent infection. During latent infection, to restrict the antiviral cytotoxic T response, EBV-encoded nuclear antigen 1 (EBNA1) maintains encoded protein sequence but biases codons used in mRNA such that the subsequent secondary structure does not include double strand stem features necessary for the antiviral response and downstream antigen presentation. The glycine-alanine repeat domain (GAr) within EBNA is responsible for translational efficiency and enhanced immune recognition. In this domain, 99% of the glycine residues and 100% of the alanine residues within the GAr domain (position 87-352 of UniprotKB P03211) are comprised of purine codons (GGG, GGA, and GCA) which is significantly more than human average glycine and alanine purine codons, 49.3% and 33.3%, respectively (Tellam). This example describes the design and making of an exemplary composition described herein to edit the sequence of EBNA1 to augment the secondary structure of the viral mRNA in order to induce an anti-viral response.

### Plasmid Construction

RNA-binding domain: The sequences of EBV E1-GAr are referenced in Tellam et al. 2008. PNAS 105.27: 9319-9324 (Table 1). In this example, to target EBV E1-Gar secondary structure, we perform site directed mutagenesis of the PUM1-HD at an 8-nucleotide sequence: GCGGGAGG, which is found in positions 20-27 of the 105-mer nucleotide sequence of the native EBNA1 GAr found in Table 1 of Tellam et al.

(5′TAAaggagcaggagcaggagcgggaggggcaggagcaggaggggc
aggagcaggaggaggggcaggagcaggaggaggggcaggaggggcagg
aggggcaggaAT-3′).

To target this sequence, mutations to be made to hPUM 1 are as follows:

Repeat 1: R864N and Q867E
Wild-type: HIMEFSQDQHGSRFIQLKLERATPAERQLVFNEILQ
Mutant: HIMEFSQDQHGSNFIELKLERATPAERQLVFNEILQ
Repeat 2: N899C and Q903R
Wild-type: AAYQLMVDVFGNYVIQKFFEFGSLEQKLALAERIRG
Mutant: AAYQLMVDVFGCYVIRKFFEFGSLEQKLALAERIRG
Repeat 3: C935S, R936N and Q939E
Wild-type: HVLSLALQMYGCRVIQKALEFIPSDQQNEMVRELDG
Mutant: AAYQLMVDVFGSNVIEKFFEFGSLEQKLALAERIRG
Repeat 4: N971S, H972N and Q975E
Wild-type: HVLKCVKDQNGNHVVQKCIECVQPQSLQFIIDAFKG
Mutant: HVLKCVKDQNGSNVVEKCIECVQPQSLQFIIDAFKG
Repeat 5: C1007S, R1008N and Q1011E
Wild-type: QVFALSTHPYGCRVIQRILEHCLPDQTLPILEELHQ
Mutant: QVFALSTHPYGSNVIERILEHCLPDQTLPILEELHQ
Repeat 6: N1043S and Y1044R
Wild-type: HTEQLVQDQYGNYVIQHVLEHGRPEDKSKIVAEIRG
Mutant: HTEQLVQDQYGSRVIQHVLEHGRPEDKSKIVAEIRG
Repeat 7: No Mutation
Wild-type: NVLVLSQHKFASNVVEKCVTHASRTERAVLIDEVCTMND
GPHS
Repeat 8: N1122S, Y1123N and Q1126E
Wild-type: ALYTMMKDQYANYVVQKMIDVAEPGQRKIVMHKIRP
HIATLRKYTYGKHILAKLEKYYMKNGVDLG
Mutant: ALYTMMKDQYASNVVEKMIDVAEPGQRKIVMHKIRP
HIATLRKYTYGKHILAKLEKYYMKNGVDLG

RNA-editing domain: the RNA-editing domain comprises a catalytic domain of human ADAR2DD and is designed and made as in Example 1.

PCR ligation and amplification of the fusion construct EBVE1-GAr.RBD-ADAR2DD is performed as described in Adamala, et al. 2016. PNAS 113.19: E2579-E2588.

### EBNA1 Expression Constructs

To generate EBNA1 expression constructs, full-length EBV-encoded EBNA1 (E1) and 102-nt increment of the EBNA1 GAr sequence (EBNA1-GA) are cloned into the expression vector pcDNA3 (Invitrogen). The expression vectors are then subcloned in-frame with a sequence coding for GFP (pEGFP-N1; Clontech) as described in Tellam.

### Cell Culture and Transfection

DG75 (ATCC) or HEK293 cells are maintained in RPMI medium 1640 supplemented with 2 mM L-glutamine, 100 units/ml penicillin, and 100 μg/ml streptomycin plus 10% FCS as previously described in Tellam, J. et al PNAS 2008.

### Transfection of EBNA1 Constructs

Cells are transfected with 10 μg of expression constructs by using the BioRad Gene Pulser (960 μF, 250 V, 0.4-cm gap electrode, 300-μl assay volume, 25° C.). 2 hours post transfection with EBV, cells are transiently transfected with 0.5 μg EBVE1-GAr.RBD-ADAR2DD gene plasmid then 4 h later, media is replaced with fresh medium. 24 hours post final transfection, cells are harvested and subjected to SDS/PAGE and immuno-blotted with either anti-GFP (1:2,000) or an actin mAb (1:1,000) as described in Tellam, J. et al PNAS 2008.

### Translation Assay

EBNA1/pcDNA3 expression constructs are linearized with XbaI and 1 μg of template transcribed with T7 RNA polymerase by using a Riboprobe in vitro transcription system (Promega) supplemented with 50 μCi [α-32P]UTP (Amersham Biosciences). For translation assays EBNA1/pcDNA3 vectors are transcribed and translated in vitro with T7 RNA polymerase by using a coupled transcription/translation reticulocyte lysate system (Promega) supplemented with 250 μCi 35[S]methionine (Amersham Biosciences). Lysates are subjected to SDS/PAGE and autoradiography as described in Tellam, J. et al PNAS 2008. Editing is confirmed by sequencing.

shape analysis

Lowest energy confirmations of edited sequences are predicted with MFOLD as described in Zuker, M. et al Nucleic Acid Res 2003.

qRT-PCR

cDNA synthesis of EBNA1 and 1 μg of isolated RNA per sample by using MMLV SuperScript III reverse transcriptase (Invitrogen) and an anchored oligo(T)18 primer combined with random hexamers. qRT-PCR using the Sybr Green-based fluorescent detection system and the ABI Prism 7900 Sequence Detection System (Applied Biosystems) is used to measure mRNA abundance. Ribosomal protein P0 (RPLP0; GenBank accession no. NM_053275) is used as the reference gene for all samples as described in Tellam, J. et al PNAS 2008.

Each qRT-PCR contains 2.5 ml of 2× Sybr Green Master Mix (Applied Biosystems), 0.25 ml of each primer giving a final concentration of 500 nM each, 1.0 ml water, and 1.0 ml of a 1/10 dilution of the stock cDNA template. The cycling conditions should be 40 cycles of 95° C. for 15 s and 60° C. for 1 min. At the completion of each run, a dissociation melt curve analysis is performed.

### Measuring Protein Synthesis

HEK293 cells are transfected with EBNA1-GFP expression constructs along with EBVE1-GAr.RBD-ADAR2DD. Twenty-four hours post transfection the cells are labeled at 37° C. for 12-14 h in growth medium containing 20 μCi/ml 3[H]methionine (Amersham Biosciences). Cells are washed in PBS and incubated in methionine-free growth medium for 30 min at 37° C. preceding a 30-min pulse with 100 μCi 35[S]methionine. Following the pulse, cells are lysed in Tris-buffered saline with 1% Triton X-100 and protease inhibitors and precleared with Protein A Sepharose, and lysates are immunoprecipitated with anti-GFP or a mAb to 0-tubulin (Sigma). Immunoprecipitated samples are added to 10 ml of scintillant fluid, Ultima Gold (PerkinElmer Life and Analytical Sciences), and counted on a Packard liquid scintillation analyzer, Tri-carb 2100TR.

### Example 6: Sequences

RNA binding domain of PUM1; Gly 828 to Gly 1176 of the amino acid sequence of GenBank:
AAG31807.1
SEQ ID NO: 1
GRSRLLEDFRNNRYPNLQLREIAGHIMEFSQDQHGSRFIQLKLERATPAERQLVFNEILQ
AAYQLMVDVFGNYVIQKFFEFGSLEQKLALAERIRGHVLSLALQMYGCRVIQKALEFIPS
DQQNEMVRELDGHVLKCVKDQNGNHVVQKCIECVQPQSLQFIIDAFKGQVFALSTHPYGC
RVIQRILEHCLPDQTLPILEELHQHTEQLVQDQYGNYVIQHVLEHGRPEDKSKIVAEIRG
NVLVLSQHKFASNVVEKCVTHASRTERAVLIDEVCTMNDGPHSALYTMMKDQYANYVVQK
MIDVAEPGQRKIVMHKIRPHIATLRKYTYGKHILAKLEKYYMKNGVDLG
ADAR2 amino acid sequence; NCBI Reference Sequence NG 052015.1
SEQ ID NO: 2
LSNGGGGGPGRKRPLEEGSNGHSKYRLKKRRKTPGPVLPKNALMQLNEIKPGLQYTLLSQ
TGPVHAPLFVMSVEVNGQVFEGSGPTKKKAKLHAAEKALRSFVQFPNASEAHLAMGRTLS
VNTDFTSDQADFPDTLFNGFETPDKAEPPFYVGSNGDDSFSSSGDLSLSASPVPASLAQP
PLPVLPPFPPPSGKNPVMILNELRPGLKYDFLSESGESHAKSFVMSVVVDGQFFEGSGRN
KKLAKARAAQSALAAIFNLHLDQTPSRQPIPSEGLQLHLPQVLADAVSRLVLGKFGDLTD
NFSSPHARRKVLAGVVMTTGTDVKDAKVISVSTGTKCINGEYMSDRGLALNDCHAEIISR
RSLLRFLYTQLELYLNNKDDQKRSIFQKSERGGFRLKENVQFHLYISTSPCGDARIFSPH
EPILEGSRSYTQAGVQWCNHGSLQPRPPGLLSDPSTSTFQGAGTTEPADRHPNRKARGQL
RTKIESGEGTIPVRSNASIQTWDGVLQGERLLTMSCSDKIARWNVVGIQGSLLSIFVEPI
YFSSIILGSLYHGDHLSRAMYQRISNIEDLPPLYTLNKPLLSGISNAEARQPGKAPNFSV
NWTVGDSAIEVINATTGKDELGRASRLCKHALYCRWMRVHGKVPSHLLRSKITKPNVYHE
SKLAAKEYQAAKARLFTAFIKAGLGAWVEKPTEQDQFSLTP (SEQ ID NO: 2)
SEQ ID NO: 3: amino acid sequence of GluA2; NCBI Reference Sequence: NM 000826.3.
MQKIMHISVL LSPVLWGLIF GVSSNSIQIG GLFPRGADQE YSAFRVGMVQ
FSTSEFRLTP HIDNLEVANS FAVTNAFCSQ FSRGVYAIFG FYDKKSVNTI
TSFCGTLHVS FITPSFPTDG THPFVIQMRP DLKGALLSLI EYYQWDKFAY
LYDSDRGLST LQAVLDSAAE KKWQVTAINV GNINNDKKDE MYRSLFQDLE
LKKERRVILD CERDKVNDIV DQVITIGKHV KGYHYIIANL GFTDGDLLKI
QFGGANVSGF QIVDYDDSLV SKFIERWSTL EEKEYPGAHT TTIKYTSALT
YDAVQVMTEA FRNLRKQRIE ISRRGNAGDC LANPAVPWGQ GVEIERALKQ
VQVEGLSGNI KFDQNGKRIN YTINIMELKT NGPRKIGYWS EVDKMVVTLT
ELPSGNDTSG LENKTVVVTT ILESPYVMMK KNHEMLEGNE RYEGYCVDLA
AEIAKHCGFK YKLTIVGDGK YGARDADTKI WNGMVGELVY GKADIAIAPL
TITLVREEVI DFSKPFMSLG ISIMIKKPQK SKPGVFSFLD PLAYEIWMCI
VFAYIGVSVV LFLVSRFSPY EWHTEEFEDG RETQSSESTN EFGIFNSLWF
SLGAFMQQGC DISPRSLSGR IVGGVWWFFT LIIISSYTAN LAAFLTVERM
VSPIESAEDL SKQTEIAYGT LDSGSTKEFF RRSKIAVFDK MWTYMRSAEP
SVFVRTTAEG VARVRKSKGK YAYLLESTMN EYIEQRKPCD TMKVGGNLDS
KGYGIATPKG SSLRNAVNLA VLKLNEQGLL DKLKNKWWYD KGECGSGGGD
SKEKTSALSL SNVAGVFYIL VGGLGLAMLV ALIEFCYKSR AEAKRMKVAK
NAQNINPSSS QNSQNFATYK EGYNVYGIES VKI

What is claimed is:

1. A polypeptide comprising: (a) an RNA binding domain comprising a plurality of (e.g., 2-50, 10-30, or 16-21) RNA base-binding motifs, each of which binds to an RNA base, and which are ordered in the RNA binding domain to bind to the consecutive order of the RNA bases in the target RNA sequence, linked to (b) a heterologous RNA editing domain.

2. A polypeptide comprising: (a) an RNA binding domain comprising a plurality of (e.g., 2-50, 10-30, or 16-21) RNA base-binding motifs, each of which binds to an RNA base, and which are ordered in the RNA binding domain to bind to the consecutive order of the RNA bases in the target RNA sequence, linked to (b) a heterologous RNA editing domain, wherein the polypeptide does not comprise a nuclease or a functional fragment thereof.

3. A polypeptide comprising: (a) an RNA binding domain comprising a plurality of (e.g., 2-50, 10-30, or 16-21) RNA base-binding motifs, each of which binds to an RNA base, and which are ordered in the RNA binding domain to bind to the consecutive order of the RNA bases in the target RNA sequence, linked to (b) a heterologous RNA editing domain comprising a catalytic domain of a deaminase or functional fragment or variant thereof.

4. A polypeptide comprising: (a) an RNA binding domain comprising a plurality of (e.g., 2-50, 10-30, or 16-21) RNA base-binding motifs, each of which binds to an RNA base, and which are ordered in the RNA binding domain to bind to the consecutive order of the RNA bases in the target RNA sequence, linked to (b) a heterologous RNA effector comprising a splicing factor.

5. The polypeptide of any preceding claim, wherein the plurality of RNA base-binding motifs comprises at least 3 (e.g., at least 4 at least 5, at least 6, at least 7, at least 8, at least 9, between 14-24, between 15-23, between 16-22, between 16-21, between 2-20, between 2-15, between 2-10, between 2-8, between 3-20, between 3-15, between 3-10, between 3-8, between 4-8, up to 25, up to 30) PUM RNA-binding motifs.

6. The polypeptide of any preceding claim, wherein the RNA binding domain binds an RNA sequence of between 2-50 nucleotides (e.g., between 14-30, 15-26, 16-21, 2-40, 2-30, 2-25, 2-20, 5-50, 5-40, 5-30, 5-25, 5-20, 5-15, 2-18, 2-15, 2-12, 2-10, 2-9, 2-8, 3-20, 3-15, 3-10, 3-9, 3-8, 4-12, 4-10, 4-9, 4-8, 5-10, 5-9, 5-8 nucleotides).

7. The polypeptide of any preceding claim, wherein the RNA binding domain is between 90-500 amino acid residues, e.g., between 90-450 amino acid residues, between 90-400 amino acid residues, between 90-350 amino acid residues, between 90-300 amino acid residues, between 120-400 amino acid residues.

8. The polypeptide of any preceding claim, wherein the RNA binding domain has at least 80% identity (e.g., at least 85% identity, at least 87% identity, at least 90% identity, at least 92% identity, at least 95% identity, at least 97% identity, at least 98% identity, or 99% identity) and less than 100% identity to a corresponding amino acid sequence of a wild type PUM-HD, e.g., wild type human PUM1-HD.

9. The polypeptide of any preceding claim, wherein the RNA binding domain binds an RNA sequence comprising a disease-associated mutation.

10. The polypeptide of any preceding claim, wherein the RNA binding domain binds an RNA sequence comprising a disease-associated mutation and the RNA editing domain edits (e.g., corrects) the disease-associated mutation.

11. The polypeptide of any preceding claim, wherein the RNA editing domain comprises a polypeptide comprising a catalytic domain of an RNA deaminase (e.g., an adenosine deaminase or a cytidine deaminase) or a functional fragment or variant thereof.

12. The polypeptide of any preceding claim, wherein the RNA editing domain comprises the catalytic domain of an Adenosine Deaminase Acting on RNA (ADAR) (e.g., human ADAR 1, human ADAR2, human ADAR3, or human ADAR4); an Adenosine Deaminase Acting on tRNAs (ADAT); a Cytosine Deaminase Acting on RNA (CDAR); or a functional fragment or variant thereof.

13. The polypeptide of claim 11 or 12, wherein the catalytic domain of the deaminase is at least 80% identical (e.g., at least 85%, 87%, 90%, 92%, 95%, 98%, 99%, 100% identical) to a sequence shown in Table B.

14. The polypeptide of any preceding claim, wherein the RNA editing domain modifies at least 1, 2, 3, 4, 5, 6, 7, 8, 9, or 10 (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10) nucleotides of the target RNA sequence or an RNA comprising the target sequence.

15. The polypeptide of any preceding claim, wherein the RNA editing domain modifies a single nucleotide of the target RNA sequence or an RNA comprising the target sequence.

16. The polypeptide of any preceding claim, wherein the RNA editing domain changes a base to another base, e.g., changes a cytosine to a uracil; an adenosine to an inosine; or a guanosine to an adenosine.

17. The polypeptide of any preceding claim, wherein the RNA editing domain modifies an amino-acid encoding sequence of the target RNA sequence.

18. The polypeptide of claim 17, wherein the modification to the amino-acid encoding sequence of the target RNA sequence alters the amino acid sequence of a product polypeptide encoded by the target RNA sequence.

19. The polypeptide of any preceding claim, wherein the RNA editing domain modifies at least 1, 2, 3, 4, 5, 6, 7, 8, 9, or 10 (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10) nucleotides of the target RNA sequence, and optionally no more than 1, 2, 3, 4, 5, 6, 7, 8, 9, or 10 nucleotides of the target RNA sequence.

20. The polypeptide of any preceding claim, wherein the RNA binding domain binds a secondary structure of an RNA.

21. The polypeptide of any preceding claim, wherein the RNA binding domain binds a pre-mRNA, e.g., an intron-exon junction of a pre-mRNA.

22. The polypeptide of any preceding claim, wherein the polypeptide inhibits (e.g., formation of), destabilizes, and/or eliminates a secondary structure of the target RNA sequence or an RNA comprising the target RNA sequence.

23. The polypeptide of any preceding claim, wherein the polypeptide alters the splicing of the target RNA sequence or an RNA comprising the target RNA sequence.

24. The polypeptide of claim 23, wherein the polypeptide inhibits, e.g., eliminates, splicing of the target RNA sequence or an RNA comprising the target RNA sequence at a splice site (e.g., a target splice site), and optionally does not inhibit splicing of the target RNA sequence or an RNA comprising the target RNA sequence at one or more other splice site(s) (e.g., one or more non-target splice site(s)).

25. The polypeptide of any preceding claim, wherein the polypeptide decreases expression of a gene, e.g., a gene encoding the target RNA sequence.

26. The polypeptide of any preceding claim, wherein the polypeptide decreases the level of a product polypeptide encoded by the target RNA sequence.

27. The polypeptide of any preceding claim, wherein the polypeptide eliminates a stop codon, e.g., a premature stop codon, in the target RNA sequence or an RNA comprising the target RNA sequence.

28. The polypeptide of any preceding claim, wherein the polypeptide creates a stop codon, e.g., a premature stop codon, in the target RNA sequence or an RNA comprising the target RNA sequence.

29. The polypeptide of any preceding claim, wherein at least 2 (e.g., 3, 4, 5, 6, 7, 8, 9 or more) of the plurality of RNA base-binding motifs of the RNA-binding domain are joined by a linker, e.g., an amino acid linker.

30. The polypeptide of any preceding claim, wherein the RNA binding domain and the RNA editing domain are linked by a linker, e.g., an amino acid linker.

31. The polypeptide of any preceding claim, wherein the polypeptide further comprises a splicing factor.

32. A composition comprising the polypeptide of any preceding claim, and an anti-sense oligonucleotide comprising a sequence that is complementary to the target RNA sequence.

33. A nucleic acid encoding a polypeptide of any preceding claim.

34. The nucleic acid of claim 33, wherein the nucleic acid is an RNA, e.g., an mRNA.

35. A composition comprising the nucleic acid of either of claim 33 or 34, and an anti-sense oligonucleotide comprising a sequence that is complementary to the target RNA sequence.

36. A composition comprising the nucleic acid of either claim 33 or 34, and a nucleic acid encoding an anti-sense oligonucleotide comprising a sequence that is complementary to the target RNA sequence.

37. An expression vector (e.g., a plasmid vector, a viral vector) comprising a nucleic acid of either of claim 33 or 34.

38. A host cell (e.g., a bacterial host cell, a mammalian host cell) comprising an exogenous polypeptide of any preceding claim, a nucleic acid of either of claim 33 or 34, a composition of either of claim 35 or 36, or a vector of claim 37.

39. A GMP-grade pharmaceutical composition comprising the polypeptide, nucleic acid, vector, composition, or host cell of any preceding claim and a pharmaceutically acceptable excipient.

40. The polypeptide, nucleic acid, vector, composition, pharmaceutical composition, or host cell of any preceding claim, encapsulated or formulated in a pharmaceutical carrier (e.g., a vesicle, liposome, LNP).

41. A method of modifying (e.g., changing the sequence of) a target RNA, comprising contacting a cell, tissue or subject with a polypeptide, nucleic acid, vector, composition, or host cell, or GMP-grade pharmaceutical composition of any preceding claim, in an amount and for a time sufficient for the RNA binding domain of the polypeptide to bind the target RNA in the cell, tissue or subject, and for the RNA editing domain of the polypeptide to edit the target RNA.

42. The method of claim 41, wherein the target RNA is a pre-mRNA or an mRNA that has secondary and/or tertiary structure.

43. The method of either of claim 41 or 42, wherein the target RNA is a pre-mRNA, e.g., an intron-exon junction of a pre-mRNA.

44. The method of any previous claim, wherein the polypeptide alters the nucleotide sequence of the target RNA.

45. The method of claim 44, wherein altering comprises modifying at least 1, 2, 3, 4, 5, 6, 7, 8, 9, or 10 (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10) nucleotides of the target RNA sequence or an RNA comprising the target sequence.

46. The method of claim 44, wherein altering comprises modifying a single nucleotide of the target RNA sequence or an RNA comprising the target sequence.

47. The method of any of claims 44-46, wherein altering comprises changing a base to another base, e.g., changes a cytosine to a uracil; an adenosine to an inosine; or a guanosine to an adenosine.

48. The method of any of claims 44-47, wherein altering comprises modifying an amino-acid encoding sequence of the target RNA sequence.

49. The method of claim 48, wherein the modification to the amino-acid encoding sequence of the target RNA sequence alters the amino acid sequence of a product polypeptide encoded by the target RNA sequence.

50. The method of any previous claim, wherein the target RNA comprises a pre-mRNA or mRNA in a cell, tissue or subject, and the polypeptide alters (e.g., increases or decreases) secondary or tertiary structure of the pre-mRNA or mRNA.

51. The method of any previous claim, wherein the target RNA comprises a pre-mRNA or mRNA in a cell, tissue or subject, and the polypeptide alters splicing of the pre-mRNA or mRNA.

52. The polypeptide of claim 51, wherein the polypeptide inhibits, e.g., eliminates, splicing of the pre-mRNA or mRNA at a splice site (e.g., a target splice site), and optionally does not inhibit splicing of the pre-mRNA or mRNA at one or more other splice site(s) (e.g., one or more non-target splice site(s)).

53. The pharmaceutical composition, polypeptide, nucleic acid, vector, composition, host cell, or method of any previous claim, wherein the target RNA comprises Epstein-Barr Virus (EBV) mRNA, e.g., EBV nuclear antigen 1 (EBNA1) mRNA.

54. The pharmaceutical composition, polypeptide, nucleic acid, vector, composition, host cell, or method of any previous claim, wherein the target RNA comprises Spinal Muscle Neuron 2 (SMN2) mRNA.

55. The pharmaceutical composition, polypeptide, nucleic acid, vector, composition, host cell, or method of any previous claim, wherein the target RNA comprises GluA2 mRNA.

56. The pharmaceutical composition, polypeptide, nucleic acid, vector, composition, host cell, or method of any previous claim, wherein the polypeptide comprises an amino acid sequence chosen from SEQ ID NOs: 13-21 or an amino acid sequence with at least 80, 85, 90, 91, 92, 93, 94, 95, 96, 97, 98, or 99% identity thereto or having no more than 1, 2, 3, 4, 5, 6, 7, 8, 9, or 10 base alterations (e.g., substitutions, deletions, or insertions) relative thereto.

57. The pharmaceutical composition, polypeptide, nucleic acid, vector, composition, host cell, or method of any previous claim, wherein the RNA-binding domain binds to a target RNA sequence comprising an RNA sequence chosen from SEQ ID NOs: 22-25 or having no more than 1, 2, 3, 4, 5, 6, 7, 8, 9, or 10 base alterations relative thereto.

58. A method of treating a disease or disorder in a subject, e.g., a human subject, comprising administering to the subject an effective amount of a polypeptide, pharmaceutical composition, nucleic acid, vector, composition, or host cell of any preceding claim, thereby treating the disease or disorder,
wherein the disease or disorder is chosen from Meier-Gorlin syndrome, Seckel syndrome 4, Joubert syndrome 5, Leber congenital amaurosis 10; Charcot-Marie-Tooth disease, type 2; Charcot-Marie-Tooth disease, type 2; Usher syndrome, type 2C; Spinocerebellar ataxia 28; Spinocerebellar ataxia 28; Spinocerebellar ataxia 28; Long QT syndrome 2; Sjogren-Larsson syndrome; Hereditary fructosuria; Hereditary fructosuria; Neuroblastoma; Neuroblastoma; Kallmann syndrome 1; Kallmann syndrome 1; Kallmann syndrome 1; Metachromatic leukodystrophy, Rett syndrome, Amyotrophic lateral sclerosis type 10, Li-Fraumeni syndrome, Cystic fibrosis, Hurler Syndrome, alpha-1-antitrypsin (AlAT) deficiency, Parkinson's disease, Alzheimer's disease, albinism, Amyotrophic lateral sclerosis, Asthma, b-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Inflammatory Bowel Disease (I BD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, Sturge-Weber Syndrome, and cancer.

59. A method of treating a subject (e.g., a human subject) infected by or suspected of being infected by Epstein-Barr Virus (EBV), comprising administering to the subject an effective amount of a polypeptide, pharmaceutical composition, nucleic acid, vector, composition, or host cell of any preceding claim, thereby treating the subject infected by or suspected of being infected by Epstein-Barr Virus (EBV).

60. The method of claim 59, wherein the subject has mononucleosis or cancer (e.g., Burkitt lymphoma, Hodgkin's, and nasopharyngeal carcinomas).

61. A method of treating a subject (e.g., a human subject) having Spinal Muscle Atrophy (SMA), comprising administering to the subject an effective amount of a polypeptide, pharmaceutical composition, nucleic acid, vector, composition, or host cell of any preceding claim, thereby treating the subject having SMA.

62. A method of treating a subject (e.g., a human subject) having Amyotrophic Lateral Sclerosis (ALS), comprising administering to the subject an effective amount of a polypeptide, pharmaceutical composition, nucleic acid, vector, composition, or host cell of any preceding claim, thereby treating the subject having ALS.
